SEQUENCE LISTING (116 sequences)

SEQ ID NO: 1 - protein, 4 aa, synthetic construct
  REGION 1..4: note = Synthetic polypeptide
PSAP

SEQ ID NO: 2 - skipped

SEQ ID NO: 3 - protein, 4 aa, synthetic construct
  REGION 1..4: note = Synthetic polypeptide
AGVF

SEQ ID NO: 4 - protein, 4 aa, synthetic construct
  REGION 1..4: note = Synthetic polypeptide
GFLG

SEQ ID NO: 5 - protein, 4 aa, synthetic construct
  REGION 1..4: note = Synthetic polypeptide
ALAL

SEQ ID NO: 6 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 7 - protein, 870 aa, Homo sapiens
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 8 - protein, 1319 aa, Homo sapiens
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 9 - protein, 955 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 10 - protein, 757 aa, Homo sapiens
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 11 - protein, 748 aa, Homo sapiens
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 12 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 13 - protein, 1606 aa, Homo sapiens
MLLHLCSVKNLYQNRFLGLAAMASPSRNSQSRRRCKEPLRYSYNPDQFHNMDLRGGPHDGVTIPRSTSDTDLVTSDSRSTLMVSSSYYSIGHSQDLVIHWDIKEEVDAGDWIGMYLIDEVLSENFLDYKNRGVNGSHRGQIIWKIDASSYFVEPETKICFKYYHGVSGALRATTPSVTVKNSAAPIFKSIGADETVQGQGSRRLISFSLSDFQAMGLKKGMFFNPDPYLKISIQPGKHSIFPALPHHGQERRSKIIGNTVNPIWQAEQFSFVSLPTDVLEIEVKDKFAKSRPIIKRFLGKLSMPVQRLLERHAIGDRVVSYTLGRRLPTDHVSGQLQFRFEITSSIHPDDEEISLSTEPESAQIQDSPMNNLMESGSGEPRSEAPESSESWKPEQLGEGSVPDGPGNQSIELSRPAEEAAVITEAGDQGMVSVGPEGAGELLAQVQKDIQPAPSAEELAEQLDLGEEASALLLEDGEAPASTKEEPLEEEATTQSRAGREEEEKEQEEEGDVSTLEQGEGRLQLRASVKRKSRPCSLPVSELETVIASACGDPETPRTHYIRIHTLLHSMPSAQGGSAAEEEDGAEEESTLKDSSEKDGLSEVDTVAADPSALEEDREEPEGATPGTAHPGHSGGHFPSLANGAAQDGDTHPSTGSESDSSPRQGGDHSCEGCDASCCSPSCYSSSCYSTSCYSSSCYSASCYSPSCYNGNRFASHTRFSSVDSAKISESTVFSSQDDEEEENSAFESVPDSMQSPELDPESTNGAGPWQDELAAPSGHVERSPEGLESPVAGPSNRREGECPILHNSQPVSQLPSLRPEHHHYPTIDEPLPPNWEARIDSHGRVFYVDHVNRTTTWQRPTAAATPDGMRRSGSIQQMEQLNRRYQNIQRTIATERSEEDSGSQSCEQAPAGGGGGGGSDSEAESSQSSLDLRREGSLSPVNSQKITLLLQSPAVKFITNPEFFTVLHANYSAYRVFTSSTCLKHMILKVRRDARNFERYQHNRDLVNFINMFADTRLELPRGWEIKTDQQGKSFFVDHNSRATTFIDPRIPLQNGRLPNHLTHRQHLQRLRSYSAGEASEVSRNRGASLLARPGHSLVAAIRSQHQHESLPLAYNDKIVAFLRQPNIFEMLQERQPSLARNHTLREKIHYIRTEGNHGLEKLSCDADLVILLSLFEEEIMSYVPLQAAFHPGYSFSPRCSPCSSPQNSPGLQRASARAPSPYRRDFEAKLRNFYRKLEAKGFGQGPGKIKLIIRRDHLLEGTFNQVMAYSRKELQRNKLYVTFVGEEGLDYSGPSREFFFLLSQELFNPYYGLFEYSANDTYTVQISPMSAFVENHLEWFRFSGRILGLALIHQYLLDAFFTRPFYKALLRLPCDLSDLEYLDEEFHQSLQWMKDNNITDILDLTFTVNEEVFGQVTERELKSGGANTQVTEKNKKEYIERMVKWRVERGVVQQTEALVRGFYEVVDSRLVSVFDARELELVIAGTAEIDLNDWRNNTEYRGGYHDGHLVIRWFWAAVERFNNEQRLRLLQFVTGTSSVPYEGFAALRGSNGLRRFCIEKWGKITSLPRAHTCFNRLDLPPYPSYSMLYEKLLTAVEETSTFGLE

SEQ ID NO: 14 - protein, 1572 aa, Homo sapiens
MASSAREHLLFVRRRNPQMRYTLSPENLQSLAAQSSMPENMTLQRANSDTDLVTSESRSSLTASMYEYTLGQAQNLIIFWDIKEEVDPSDWIGLYHIDENSPANFWDSKNRGVTGTQKGQIVWRIEPGPYFMEPEIKICFKYYHGISGALRATTPCITVKNPAVMMGAEGMEGGASGNLHSRKLVSFTLSDLRAVGLKKGMFFNPDPYLKMSIQPGKKSSFPTCAHHGQERRSTIISNTTNPIWHREKYSFFALLTDVLEIEIKDKFAKSRPIIKRFLGKLTIPVQRLLERQAIGDQMLSYNLGRRLPADHVSGYLQFKVEVTSSVHEDASPEAVGTILGVNSVNGDLGSPSDDEDMPGSHHDSQVCSNGPVSEDSAADGTPKHSFRTSSTLEIDTEELTSTSSRTSPPRGRQDSLNDYLDAIEHNGHSRPGTATCSERSMGASPKLRSSFPTDTRLNAMLHIDSDEEDHEFQQDLGYPSSLEEEGGLIMFSRASRADDGSLTSQTKLEDNPVENEEASTHEAASFEDKPENLPELAESSLPAGPAPEEGEGGPEPQPSADQGSAELCGSQEVDQPTSGADTGTSDASGGSRRAVSETESLDQGSEPSQVSSETEPSDPARTESVSEASTRPEGESDLECADSSCNESVTTQLSSVDTRCSSLESARFPETPAFSSQEEEDGACAAEPTSSGPAEGSQESVCTAGSLPVVQVPSGEDEGPGAESATVPDQEELGEVWQRRGSLEGAAAAAESPPQEEGSAGEAQGTCEGATAQEEGATGGSQANGHQPLRSLPSVRQDVSRYQRVDEALPPNWEARIDSHGRIFYVDHVNRTTTWQRPTAPPAPQVLQRSNSIQQMEQLNRRYQSIRRTMTNERPEENTNAIDGAGEEADFHQASADFRRENILPHSTSRSRITLLLQSPPVKFLISPEFFTVLHSNPSAYRMFTNNTCLKHMITKVRRDTHHFERYQHNRDLVGFLNMFANKQLELPRGWEMKHDHQGKAFFVDHNSRTTTFIDPRLPLQSSRPTSALVHRQHLTRQRSHSAGEVGEDSRHAGPPVLPRPSSTFNTVSRPQYQDMVPVAYNDKIVAFLRQPNIFEILQERQPDLTRNHSLREKIQFIRTEGTPGLVRLSSDADLVMLLSLFEEEIMSYVPPHALLHPSYCQSPRGSPVSSPQNSPGTQRANARAPAPYKRDFEAKLRNFYRKLETKGYGQGPGKLKLIIRRDHLLEDAFNQIMGYSRKDLQRNKLYVTFVGEEGLDYSGPSREFFFLVSRELFNPYYGLFEYSANDTYTVQISPMSAFVDNHHEWFRFSGRILGLALIHQYLLDAFFTRPFYKALLRILCDLSDLEYLDEEFHQSLQWMKDNDIHDILDLTFTVNEEVFGQITERELKPGGANIPVTEKNKKEYIERMVKWRIERGVVQQTESLVRGFYEVVDARLVSVFDARELELVIAGTAEIDLSDWRNNTEYRGGYHDNHIVIRWFWAAVERFNNEQRLRLLQFVTGTSSIPYEGFASLRGSNGPRRFCVEKWGKITALPRAHTCFNRLDLPPYPSFSMLYEKLLTAVEETSTFGLE

SEQ ID NO: 15 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 16 - protein, 434 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQKVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 17 - protein, 433 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 18 - protein, 65 aa, Homo sapiens
PLPPGWEQRVDQHGRVYYVDHVEKRTTWDRPEPLPPGWERRVDNMGRIYYVDHFTRTTTWQRPTL

SEQ ID NO: 19 - protein, 185 aa, Homo sapiens
PLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRT

SEQ ID NO: 20 - protein, 390 aa, Mus musculus
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 21 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 22 - protein, 391 aa, Rattus norvegicus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTAAGPPNTSYLPSMPSGISAYPSGYPPNPSGYPGCPYPPAGPYPATTSSQYPSQPPVTTAGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 23 - DNA, 3475 bp, Homo sapiens
  source 1..3475: mol_type = genomic DNA
gaattcgcggccgcgtcgaccgcttctgtggccacggcagatgaaacagaaaggctaaagagggctggagtcaggggacttctcttccaccagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggaaaactgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccaggctgggaaaaacgcacagatccccgaggcaggttttactatgtggatcacaatactcggaccaccacctggcagcgtccgaccgcggagtacgtgcgcaactatgagcagtggcagtcgcagcggaatcagctccagggggccatgcagcacttcagccaaagattcctataccagttttggagtgcttcgactgaccatgatcccctgggccccctccctcctggttgggagaaaagacaggacaatggacgggtgtattacgtgaaccataacactcgcacgacccagtgggaggatccccggacccaggggatgatccaggaaccagctttgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctttacatcatcatgcgtggcgaggagggcctggactatgggggcatcgccagagagtggtttttcctcctgtctcacgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacggaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggacgggccctggggctaggccatctctgcctgcctccctagcaggcgccagcggtggaggctgagtcgcaggacacatgccggccagttaattcattctcagcaaatgaaggtttgtctaagctgcctgggtatccacgggacaaaaacagcaaactccctccagactttgtccatgttataaacttgaaagttggttgttgtttgttaggtttgccaggtttttttgtttacgcctgctgtcactttcctgtc

SEQ ID NO: 24 - DNA, 3475 bp, Homo sapiens; identical to SEQ ID NO: 23
  source 1..3475: mol_type = genomic DNA

SEQ ID NO: 25 - DNA, 2914 bp, Homo sapiens
  source 1..2914: mol_type = genomic DNA
acagttgcctgccctgggcgggggcgagcgcgtccggtttgctggaagcgttcggaaatggcaacttgcgcggtggaggtgttcgggctcctggaggacgaggaaaattcacgaattgtgagagtaagagttatagccggaataggccttgccaagaaggatatattgggagctagtgatccttacgtgagagtgacgttatatgacccaatgaatggagttcttacaagtgtgcaaacaaaaaccattaaaaagagtttgaatccaaagtggaatgaagaaatattattcagagttcatcctcagcagcaccggcttctttttgaagtgtttgacgaaaaccgattgacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccaagagtcttccgagaactgggaaattataagagaagatgaagccaccatgtatagcagccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggaccgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgac

SEQ ID NO: 26 - DNA, 2868 bp, Homo sapiens
  source 1..2868: mol_type = genomic DNA
atggcgaccgggctcggggagccggtctatggactttccgaagacgagggagagtcccgtattctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgccttcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaa

SEQ ID NO: 27 - DNA, 2274 bp, Homo sapiens
  source 1..2274: mol_type = genomic DNA
atgtcgaaccccgggacacgcaggaacggctccagcatcaagatccgtctgacagtgttatgtgccaagaaccttgcaaagaaagacttcttcaggctccctgacccttttgcaaagattgtcgtggatgggtctgggcagtgccactcaaccgacactgtgaaaaacacattggacccaaagtggaaccagcactatgatctatatgttgggaaaacggattcgataaccattagcgtgtggaaccataagaaaattcacaagaaacagggagctggcttcctgggctgtgtgcggctgctctccaatgccatcagcagattaaaagataccggataccagcgtttggatctatgcaaactaaacccctcagatactgatgcagttcgtggccagatagtggtcagtttacagacacgagacagaataggaaccggcggctcggtggtggactgcagaggactgttagaaaatgaaggaacggtgtatgaagactccgggcctgggaggccgctcagctgcttcatggaggaaccagccccttacacagatagcaccggtgctgctgctggaggagggaattgcaggttcgtggagtccccaagtcaagatcaaagacttcaggcacagcggcttcgaaaccctgatgtgcgaggttcactacagacgccccagaaccgaccacacggccaccagtccccggaactgcccgaaggctacgaacaaagaacaacagtccagggccaagtttactttttgcatacacagactggagttagcacgtggcacgaccccaggataccaagtccctcggggaccattcctgggggagatgcagcttttctatacgaattccttctacaaggccatacatctgagcccagagaccttaacagtgtgaactgtgatgaacttggaccactgccgccaggctgggaagtcagaagtacagtttctgggaggatatattttgtagatcataataaccgaacaacccagtttacagacccaaggttacaccacatcatgaatcaccagtgccaactcaaggagcccagccagccgctgccactgcccagtgagggctctctggaggacgaggagcttcctgcccagagatacgaaagagatctagtccagaagctgaaagtcctcagacacgaactgtcgcttcagcagccccaagctggtcattgccgcatcgaagtgtccagagaagaaatctttgaggagtcttaccgccagataatgaagatgcgaccgaaagacttgaaaaaacggctgatggtgaaattccgtggggaagaaggtttggattacggtggtgtggccagggagtggctttacttgctgtgccatgaaatgctgaatccttattacgggctcttccagtattctacggacaatatttacatgttgcaaataaatccggattcttcaatcaaccccgaccacttgtcttatttccactttgtggggcggatcatggggctggctgtgttccatggacactacatcaacgggggcttcacagtgcccttctacaagcagctgctggggaagcccatccagctctcagatctggaatctgtggacccagagctgcataagagcttggtgtggatcctagagaacgacatcacgcctgtactggaccacaccttctgcgtggaacacaacgccttcgggcggatcctgcagcatgaactgaaacccaatggcagaaatgtgccagtcacagaggagaataagaaagaatacgtccggttgtatgtaaactggaggtttatgagaggaatcgaagcccagttcttagctctgcagaaggggttcaatgagctcatccctcaacatctgctgaagccttttgaccagaaggaactggagctgatcataggcggcctggataaaatagacttgaacgactggaagtcgaacacgcggctgaagcactgtgtggccgacagcaacatcgtgcggtggttctggcaagcggtggagacgttcgatgaagaaaggagggccaggctcctgcagtttgtgactgggtccacgcgagtcccgctccaaggcttcaaggctttgcaaggttctacaggcgcggcagggccccggctgttcaccatccacctgatagacgcgaacacagacaaccttccgaaggcccatacctgctttaaccggatcgacattccaccatatgagtcctatgagaagctctacgagaagctgctgacagccgtggaggagacctgcgggtttgctgtggagtga

SEQ ID NO: 28 - DNA, 2247 bp, Homo sapiens
  source 1..2247: mol_type = genomic DNA
atgtctaaccccggacgccggaggaacgggcccgtcaagctgcgcctgacagtactctgtgcaaaaaacctggtgaaaaaggattttttccgacttcctgatccatttgctaaggtggtggttgatggatctgggcaatgccattctacagatactgtgaagaatacgcttgatccaaagtggaatcagcattatgacctgtatattggaaagtctgattcagttacgatcagtgtatggaatcacaagaagatccataagaaacaaggtgctggatttctcggttgtgttcgtcttctttccaatgccatcaaccgcctcaaagacactggttatcagaggttggatttatgcaaactcgggccaaatgacaatgatacagttagaggacagatagtagtaagtcttcagtccagagaccgaataggcacaggaggacaagttgtggactgcagtcgtttatttgataacgatttaccagacggctgggaagaaaggagaaccgcctctggaagaatccagtatctaaaccatataacaagaactacgcaatgggagcgcccaacacgaccggcatccgaatattctagccctggcagacctcttagctgctttgttgatgagaacactccaattagtggaacaaatggtgcaacatgtggacagtcttcagatcccaggctggcagagaggagagtcaggtcacaacgacatagaaattacatgagcagaacacatttacatactcctccagacctaccagaaggctatgaacagaggacaacgcaacaaggccaggtgtatttcttacatacacagactggtgtgagcacatggcatgatccaagagtgcccagggatcttagcaacatcaattgtgaagagcttggtccattgcctcctggatgggagatccgtaatacggcaacaggcagagtttatttcgttgaccataacaacagaacaacacaatttacagatcctcggctgtctgctaacttgcatttagttttaaatcggcagaaccaattgaaagaccaacagcaacagcaagtggtatcgttatgtcctgatgacacagaatgcctgacagtcccaaggtacaagcgagacctggttcagaaactaaaaattttgcggcaagaactttcccaacaacagcctcaggcaggtcattgccgcattgaggtttccagggaagagatttttgaggaatcatatcgacaggtcatgaaaatgagaccaaaagatctctggaagcgattaatgataaaatttcgtggagaagaaggccttgactatggaggcgttgccagggaatggttgtatctcttgtcacatgaaatgttgaatccatactatggcctcttccagtattcaagagatgatatttatacattgcagatcaatcctgattctgcagttaatccggaacatttatcctatttccactttgttggacgaataatgggaatggctgtgtttcatggacattatattgatggtggtttcacattgcctttttataagcaattgcttgggaagtcaattaccttggatgacatggagttagtagatccggatcttcacaacagtttagtgtggatacttgagaatgatattacaggtgttttggaccataccttctgtgttgaacataatgcatatggtgaaattattcagcatgaacttaaaccaaatggcaaaagtatccctgttaatgaagaaaataaaaaagaatatgtcaggctctatgtgaactggagatttttacgaggcattgaggctcaattcttggctctgcagaaaggatttaatgaagtaattccacaacatctgctgaagacatttgatgagaaggagttagagctcattatttgtggacttggaaagatagatgttaatgactggaaggtaaacacccggttaaaacactgtacaccagacagcaacattgtcaaatggttctggaaagctgtggagttttttgatgaagagcgacgagcaagattgcttcagtttgtgacaggatcctctcgagtgcctctgcagggcttcaaagcattgcaaggtgctgcaggcccgagactctttaccatacaccagattgatgcctgcactaacaacctgccgaaagcccacacttgcttcaatcgaatagacattccaccctatgaaagctatgaaaagctatatgaaaagctgctaacagccattgaagaaacatgtggatttgctgtggaatga

SEQ ID NO: 29 - DNA, 2869 bp, Homo sapiens
  source 1..2869: mol_type = genomic DNA
ggagtcgccgccgccccgagttccggtaccatgcatttcacggtggccttgtggagacaacgccttaacccaaggaagtgactcaaactgtgagaactccaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttaaaaaaaaaaaaaaaaaaa

SEQ ID NO: 30 - DNA, 5169 bp, Homo sapiens
  source 1..5169: mol_type = genomic DNA
gcgcatcaggcgctgttgttggagccggaacaccgtgcgactctgaccgaaccggccccctcctcgcgcacacactcgccgagccgcgcgcgcccctccgccgtgacagtggccgtggcctccgctctctcggggcacccggcagccagagcgcagcgagagcgggcggtcgccagggtcccctccccagccagtcccaggcgcccggtgcactatgcggggcacgtgcgccccccagctctaatctgcgcgctgacaggagcatgatctgtgcccaggccagggctgccaaggaattgatgcgcgtacacgtggtgggtcattatgctgctacacctgtgtagtgtgaagaatctgtaccagaacaggtttttaggcctggccgccatggcgtctccttctagaaactcccagagccgacgccggtgcaaggagccgctccgatacagctacaaccccgaccagttccacaacatggacctcaggggcggcccccacgatggcgtcaccattccccgctccaccagcgacactgacctggtcacctcggacagccgctccacgctcatggtcagcagctcctactattccatcgggcactctcaggacctggtcatccactgggacataaaggaggaagtggacgctggggactggattggcatgtacctcattgatgaggtcttgtccgaaaactttctggactataaaaaccgtggagtcaatggttctcatcggggccagatcatctggaagatcgatgccagctcgtactttgtggaacctgaaactaagatctgcttcaaatactaccatggagtgagtggggccctgcgagcaaccacccccagtgtcacggtcaaaaactcggcagctcctatttttaaaagcattggtgctgatgagaccgtccaaggacaaggaagtcggaggctgatcagcttctctctctcagatttccaagccatggggttgaagaaagggatgtttttcaacccagacccttatctgaagatttccattcagcctgggaaacacagcatcttccccgccctccctcaccatggacaggagaggagatccaagatcataggcaacaccgtgaaccccatctggcaggccgagcaattcagttttgtgtccttgcccactgacgtgctggaaattgaggtgaaggacaagtttgccaagagccgccccatcatcaagcgcttcttgggaaagctgtcgatgcccgttcaaagactcctggagagacacgccataggggatagggtggtcagctacacacttggccgcaggcttccaacagatcatgtgagtggacagctgcaattccgatttgagatcacttcctccatccacccagatgatgaggagatttccctgagtaccgagcctgagtcagcccaaattcaggacagccccatgaacaacctgatggaaagcggcagtggggaacctcggtctgaggcaccagagtcctctgagagctggaagccagagcagctgggtgagggcagtgtccccgatggtccagggaaccaaagcatagagctttccagaccagctgaggaagcagcagtcatcacggaggcaggagaccagggcatggtctctgtgggacctgaaggggctggggagctcctggcccaggtgcaaaaggacatccagcctgcccccagtgcagaagagctggccgagcagctggacctgggtgaggaggcatcagcactgctgctggaagacggtgaagccccagccagcaccaaggaggagcccttggaggaggaagcaacgacccagagccgggctggaagggaagaagaggagaaggagcaggaggaggagggagatgtgtctaccctggagcagggagagggcaggctgcagctgcgggcctcggtgaagagaaaaagcaggccctgctccttgcctgtgtccgagctggagacggtgatcgcgtcagcctgcggggaccccgagaccccgcggacacactacatccgcatccacaccctgctgcacagcatgccctccgcccagggcggcagcgcggcagaggaggaggacggcgcggaggaggagtccaccctcaaggactcctcggagaaggatgggctcagcgaggtggacacggtggccgctgacccgtctgccctggaagaggacagagaagagcccgagggggctactccaggcacggcgcaccctggccactccgggggccacttccccagcctggccaatggcgcggcccaggatggcgacacgcaccccagcaccgggagcgagagcgactccagccccaggcaaggcggggaccacagttgcgagggctgtgacgcgtcctgctgcagcccctcgtgctacagctcctcgtgctacagcacgtcctgctacagcagctcgtgctacagcgcctcgtgctacagcccctcctgctacaacggcaacaggttcgccagccacacgcgcttctcctccgtggacagcgccaagatctccgagagcacggtcttctcctcgcaagacgacgaggaggaggagaacagcgcgttcgagtcggtacccgactccatgcagagccctgagctggacccggagtccacgaacggcgctgggccgtggcaagacgagctggccgcccctagcgggcacgtggaaagaagcccggaaggtctggaatcccccgtggcaggtccaagcaatcggagagaagactgggaagctcgaattgacagccacgggcgggtcttttatgtggaccacgtgaaccgcacaaccacctggcagcgtccgacggcagcagccaccccggatggcatgcggagatcggggtccatccagcagatggagcaactcaacaggcggtatcaaaacattcagcgaaccattgcaacagagaggtccgaagaagattctggcagccaaagctgcgagcaagccccagcaggaggaggcggaggtggagggagtgactcagaagccgaatcttcccagtccagcttagatctaaggagagaggggtcactttctccagtgaactcacaaaaaatcaccttgctgctgcagtccccagcggtcaagttcatcaccaaccccgagttcttcactgtgctacacgccaattatagtgcctaccgagtcttcaccagtagcacctgcttaaagcacatgattctgaaagtccgacgggatgctcgcaattttgaacgctaccagcacaaccgggacttggtgaatttcatcaacatgttcgcagacactcggctggaactgccccggggctgggagatcaaaacggaccagcagggaaagtcttttttcgtggaccacaacagtcgagctaccactttcattgacccccgaatccctcttcagaacggtcgtcttcccaatcatctaactcaccgacagcacctccagaggctccgaagttacagcgccggagaggcctcagaagtttctagaaacagaggagcctctttactggccaggccaggacacagcttagtagctgctattcgaagccaacatcaacatgagtcattgccactggcatataatgacaagattgtggcatttcttcgccagccaaacatttttgaaatgctgcaagagcgtcagccaagcttagcaagaaaccacacactcagggagaaaatccattacattcggactgagggtaatcacgggcttgagaagttgtcctgtgatgcggatctggtcattttgctgagtctctttgaagaagagattatgtcctacgtccccctgcaggctgccttccaccctgggtatagcttctctccccgatgttcaccctgttcttcacctcagaactccccaggtttacagagagccagtgcaagagccccttccccctaccgaagagactttgaggccaagctccgcaatttctacagaaaactggaagccaaaggatttggtcagggtccggggaaaattaagctcattattcgccgggatcatttgttggagggaaccttcaatcaggtgatggcctattcgcggaaagagctccagcgaaacaagctctacgtcacctttgttggagaggagggcctggactacagtggcccctcgcgggagttcttcttccttctgtctcaggagctcttcaacccttactatggactctttgagtactcggcaaatgatacttacacggtgcagatcagccccatgtccgcatttgtagaaaaccatcttgagtggttcaggtttagcggtcgcatcctgggtctggctctgatccatcagtaccttcttgacgctttcttcacgaggcccttctacaaggcactcctgagactgccctgtgatttgagtgacctggaatatttggatgaggaattccaccagagtttgcagtggatgaaggacaacaacatcacagacatcttagacctcactttcactgttaatgaagaggtttttggacaggtcacggaaagggagttgaagtctggaggagccaacacacaggtgacggagaaaaacaagaaggagtacatcgagcgcatggtgaagtggcgggtggagcgcggcgtggtacagcagaccgaggcgctggtgcgcggcttctacgaggttgtagactcgaggctggtgtccgtgtttgatgccagggagctggagctggtgatagctggcaccgcggaaatcgacctaaatgactggcggaataacactgagtaccggggaggttaccacgatgggcatcttgtgatccgctggttctgggctgcggtggagcgcttcaataatgagcagaggctgagattactgcagtttgtcacgggaacatccagcgtgccctacgaaggcttcgcagccctccgtgggagcaatgggcttcggcgcttctgcatagagaaatgggggaaaattacttctctccccagggcacacacatgcttcaaccgactggatcttccaccgtatccctcgtactccatgttgtatgaaaagctgttaacagcagtagaggaaaccagcacctttggacttgagtgaggacatggaacctcgcctgacattttcctggccagtgacatcacccttcctgggatgatccccttttccctttcccttaatcaactctcctttgattttggtattccatgatttttattttcaaac

SEQ ID NO: 31 - DNA, 2715 bp, Homo sapiens
  source 1..2715: mol_type = genomic DNA
agagttccatcagagcctgcagtggatgaaagacaatgatatccatgacatcctagacctcacgttcactgtgaacgaagaagtatttgggcagataactgaacgagaattaaagccagggggtgccaatatcccagttacagagaagaacaagaaggagtacatcgagaggatggtgaagtggaggattgagaggggtgttgtacagcaaacagagagcttagtgcgtggcttctatgaggtggtggatgccaggctggtatctgtttttgatgcaagagaactggaattggtcatcgcaggcacagctgaaatagacctaagtgattggagaaacaacacagaatatagaggaggataccatgacaatcatattgtaattcggtggttctgggctgcagtggaaagattcaacaatgaacaacgactaaggttgttacagtttgttacaggcacatccagcattccctatgaaggatttgcttcactccgagggagtaacggcccaagaagattctgtgtggagaaatgggggaaaatcactgctcttcccagagcgcatacatgttttaaccgtctggatctgcctccctacccatccttttccatgctttatgaaaaactgttgacagcagttgaagaaaccagtacttttggacttgagtgacctggaagctgaatgcccatctctgtggacaggcagtttcagaagctgccttctagaagaatgattgaacattggaagtttcaagaggatgcttcctttaggataaagctacgtgctgttgttttccaggaacaagtgctctgtcacatttggggactggagatgagtcctcttggaaggatttgggtgagcttgatgcccagggaacaacccaaccgtctttcaatcaacagttcttgactgccaaactttttccatttgttatgttccaagacaaagatgaacccatacatgatcagctccacggtaatttttagggactcaggagaatcttgaaacttacccttgaacgtggttcaagccaaactggcagcatttggcccaatctccaaattagagcaagttaaataatataataaaagtaaatatatttcctgaaagtacattcatttaagccctaagttataacagaatattcatttcttgcttatgagtgcctgcatggtgtgcaccataggtttccgctttcatgggacatgagtgaaaatgaaaccaagtcaatatgaggtacctttacagatttgcaataagatggtctgtgacaatgtatatgcaagtggtatgtgtgtaattatggctaaagacaaaccattattcagtgaattactaatgacagattttatgctttataatgcatgaaaacaattttaaaataactagcaattaatcacagcatatcaggaaaaagtacacagtgagttctgtttattttttgtaggctcattatgtttatgttctttaagatgtatataagaacctacttatcatgctgtatgtatcactcattccattttcatgttccatgcatactcgggcatcatgctaatatgtatccttttaagcactctcaaggaaacaaaagggccttttatttttataaaggtaaaaaaaattccccaaatattttgcactgaatgtaccaaaggtgaagggacattacaatatgactaacagcaactccatcacttgagaagtataatagaaaatagcttctaaatcaaacttccttcacagtgccgtgtctaccactacaaggactgtgcatctaagtaataattttttaagattcactatatgtgatagtatgatatgcatttatttaaaatgcattagactctcttccatccatcaaatactttacaggatggcatttaatacagatatttcgtatttcccccactgctttttatttgtacagcatcattaaacactaagctcagttaaggagccatcagcaacactgaagagatcagtagtaagaattccattttccctcatcagtgaagacaccacaaattgaaactcagaactatatttctaagcctgcattttcactgatgcataattttcttattaatattaagagacagtttttctatggcatctccaaaactgcatgacatcactagtcttacttctgcttaattttatgagaaggtattcttcattttaattgcttttgggattactccacatctttgtttatttcttgactaatcagattttcaatagagtgaagttaaattgggggtcataaaagcattggattgacatatggtttgccagcctatgggtttacaggcattgcccaaacatttctttgagatctatatttataagcagccatggaattcctattatgggatgttggcaatcttacattttatagaggtcatatgcatagttttcataggtgttttgtaagaactgattgctctcctgtgagttaagctatgtttactactgggaccctcaagaggaataccacttatgttacactcctgcactaaaggcacgtactgcagtgtgaagaaatgttctgaaaaagggttatagaaatctggaaataagaaaggaagagctctctgtattctataattggaagagaaaaaaagaaaaacttttaactggaaatgttagtttgtacttattgatcatgaatacaagtatatatttaattttgcaaaaaaaaaaaaaaaaaaaaaaag

SEQ ID NO: 32 - DNA, 195 bp, Homo sapiens
  source 1..195: mol_type = genomic DNA
cccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactg

SEQ ID NO: 33 - DNA, 555 bp, Homo sapiens
  source 1..555: mol_type = genomic DNA
cccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcaca

SEQ ID NO: 34 - protein, 5 aa, synthetic construct
  REGION 1..5: note = Synthetic polypeptide
ALALA

SEQ ID NO: 35 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 36 - protein, 34 aa, Homo sapiens
ETLPSGWEQRKDPHGRTYYVDHNTRTTTWERPQP

SEQ ID NO: 37 - protein, 34 aa, Homo sapiens
QPLPPGWERRVDDRRRVYYVDHNTRTTTWQRPTM

SEQ ID NO: 38 - protein, 39 aa, Homo sapiens
ENDPYGPLPPGWEKRVDSTDRVYFVNHNTKTTQWEDPRT

SEQ ID NO: 39 - protein, 34 aa, Homo sapiens
EPLPEGWEIRYTREGVRYFVDHNTRTTTFKDPRN

SEQ ID NO: 40 - protein, 34 aa, Homo sapiens
DALPAGWEQRELPNGRVYYVDHNTKTTTWERPLP

SEQ ID NO: 41 - protein, 33 aa, Homo sapiens
PLPPGWEKRTDPRGRFYYVDHNTRTTTWQRPTA

SEQ ID NO: 42 - protein, 37 aa, Homo sapiens
HDPLGPLPPGWEKRQDNGRVYYVNHNTRTTQWEDPRT

SEQ ID NO: 43 - protein, 34 aa, Homo sapiens
PALPPGWEMKYTSEGVRYFVDHNTRTTTFKDPRP

SEQ ID NO: 44 - protein, 34 aa, Homo sapiens
SPLPPGWEERQDILGRTYYVNHESRRTQWKRPTP

SEQ ID NO: 45 - protein, 34 aa, Homo sapiens
SGLPPGWEEKQDERGRSYYVDHNSRTTTWTKPTV

SEQ ID NO: 46 - protein, 34 aa, Homo sapiens
GFLPKGWEVRHAPNGRPFFIDHNTKTTTWEDPRL

SEQ ID NO: 47 - protein, 34 aa, Homo sapiens
GPLPPGWEERTHTDGRIFYINHNIKRTQWEDPRL

SEQ ID NO: 48 - protein, 34 aa, Homo sapiens
PELPEGYEQRTTVQGQVYFLHTQTGVSTWHDPRI

SEQ ID NO: 49 - protein, 34 aa, Homo sapiens
GPLPPGWEVRSTVSGRIYFVDHNNRTTQFTDPRL

SEQ ID NO: 50 - protein, 34 aa, Homo sapiens
NDLPDGWEERRTASGRIQYLNHITRTTQWERPTR

SEQ ID NO: 51 - protein, 34 aa, Homo sapiens
PDLPEGYEQRTTQQGQVYFLHTQTGVSTWHDPRV

SEQ ID NO: 52 - protein, 34 aa, Homo sapiens
GPLPPGWEIRNTATGRVYFVDHNNRTTQFTDPRL

SEQ ID NO: 53 - protein, 34 aa, Homo sapiens
APLPPGWEQRVDQHGRVYYVDHVEKRTTWDRPEP

SEQ ID NO: 54 - protein, 34 aa, Homo sapiens
EPLPPGWERRVDNMGRIYYVDHFTRTTTWQRPTL

SEQ ID NO: 55 - protein, 34 aa, Homo sapiens
GPLPPGWEKRTDSNGRVYFVNHNTRITQWEDPRS

SEQ ID NO: 56 - protein, 34 aa, Homo sapiens
KPLPEGWEMRFTVDGIPYFVDHNRRTTTYIDPRT

SEQ ID NO: 57 - protein, 33 aa, Homo sapiens
PLPPNWEARIDSHGRVFYVDHVNRTTTWQRPTA

SEQ ID NO: 58 - protein, 34 aa, Homo sapiens
LELPRGWEIKTDQQGKSFFVDHNSRATTFIDPRI

SEQ ID NO: 59 - protein, 34 aa, Homo sapiens
EALPPNWEARIDSHGRIFYVDHVNRTTTWQRPTA

SEQ ID NO: 60 - protein, 34 aa, Homo sapiens
LELPRGWEMKHDHQGKAFFVDHNSRTTTFIDPRL

SEQ ID NO: 61 - protein, 27 aa, Homo sapiens
GWEEKVDNLGRTYYVNHNNRTTQWHRP

SEQ ID NO: 62 - protein, 29 aa, Homo sapiens
PSGWEERKDAKGRTYYVNHNNRTTTWTRP

SEQ ID NO: 63 - protein, 31 aa, Homo sapiens
PPGWEMRIAPNGRPFFIDHNTKTTTWEDPRL

SEQ ID NO: 64 - protein, 31 aa, Homo sapiens
PPGWEERIHLDGRTFYIDHNSKITQWEDPRL

SEQ ID NO: 65 - protein, 101 aa, synthetic construct
  REGION 1..101: note = Synthetic polypeptide
MEPVDPRLEPWKHPGSQPRTPCTTCYCKKCCFHCQVCFTTKALGISYGRKKRRQRRRPPQGSQTHQVSLSKQPSSQPRGDQTGPKESKKKVERETEADPKP

SEQ ID NO: 66 - protein, 86 aa, synthetic construct
  REGION 1..86: note = Synthetic polypeptide
MEPVDPRLEPWKHPGSQPRTPCTTCYCKKCCFHCQVCFTTKALGISYGRKKRRQRRRPPQGSQTHQVSLSKQPSSQPRGDQTGPKE

SEQ ID NO: 67 - protein, 36 aa, synthetic construct
  REGION 1..36: note = Synthetic polypeptide
CFTTKALGISYGRKKRRQRRRPPQGSQTHQVSLSKQ

SEQ ID NO: 68 - protein, 45 aa, synthetic construct
  REGION 1..45: note = Synthetic polypeptide
MEPVDPRLEPWKHPGSQPRTPCTTCYCKKCCFHCQVCFTTKALGI

SEQ ID NO: 69 - protein, 38 aa, synthetic construct
  REGION 1..38: note = Synthetic polypeptide
RKKRRQRRRPPQGSQTHQVSLSKQPSSQPRGDQTGPKE

SEQ ID NO: 70 - protein, 35 aa, synthetic construct
  REGION 1..35: note = Synthetic polypeptide
RRQRRRPPQGSQTHQVSLSKQPSSQPRGDQTGPKE

SEQ ID NO: 71 - protein, 32 aa, synthetic construct
  REGION 1..32: note = Synthetic polypeptide
RRRPPQGSQTHQVSLSKQPSSQPRGDQTGPKE

SEQ ID NO: 72 - protein, 29 aa, synthetic construct
  REGION 1..29: note = Synthetic polypeptide
PPQGSQTHQVSLSKQPSSQPRGDQTGPKE

SEQ ID NO: 73 - protein, 9 aa, synthetic construct
  REGION 1..9: note = Synthetic polypeptide
RKKRRQRRR

SEQ ID NO: 74 - protein, 11 aa, synthetic construct
  REGION 1..11: note = Synthetic polypeptide
RKKRRQRRRPP

SEQ ID NO: 75 - protein, 13 aa, synthetic construct
  REGION 1..13: note = Synthetic polypeptide
RKKRRQRRRPPQG

SEQ ID NO: 76 - protein, 15 aa, synthetic construct
  REGION 1..15: note = Synthetic polypeptide
RKKRRQRRRPPQGSQ

SEQ ID NO: 77 - protein, 17 aa, synthetic construct
  REGION 1..17: note = Synthetic polypeptide
RKKRRQRRRPPQGSQTH

SEQ ID NO: 78 - protein, 21 aa, synthetic construct
  REGION 1..21: note = Synthetic polypeptide
CFTTKALGISYGRKKRRQRRR

SEQ ID NO: 79 - protein, 27 aa, synthetic construct
  REGION 1..27: note = Synthetic polypeptide
CFTTKALGISYGRKKRRQRRRPPQGSQ

SEQ ID NO: 80 - protein, 12 aa, synthetic construct
  REGION 1..12: note = Synthetic polypeptide
GRRKKRRQRRRP

SEQ ID NO: 81 - protein, 17 aa, synthetic construct
  REGION 1..17: note = Synthetic polypeptide
RKKRRQRRRPPQGSQTH

SEQ ID NO: 82 - protein, 130 aa, synthetic construct
  REGION 1..130: note = Synthetic polypeptide
METPLKAPEGSLGSYNEPSSCTSEQDAAAQGLVSPGDEILYQLYQPLEACDNKCYCKKCCYHCQMCFLNKGLGIWYERKGRRRRTPKKTKAHSSSASDKSISTRTGNSQPEKKQKKTLETALETIGGPGR

SEQ ID NO: 83 - protein, 103 aa, synthetic construct
  REGION 1..103: note = Synthetic polypeptide
MPGPWVAMIMLPQPKESFGGKPIGWLFWNTCKGPRRDCPHCCCPICSWHCQLCFLQKNLGINYGSGPRRRGTRGKGRRIRRTASGGDQRREADSQRSFTNMDQ

SEQ ID NO: 84 - protein, 17 aa, synthetic construct
  REGION 1..17: note = Synthetic polypeptide
SGPRPRGTRGKGRRIRR

SEQ ID NO: 85 - DNA, 48 bp, synthetic construct
  misc_feature 1..48: note = Synthetic polynucleotide
  source 1..48: mol_type = other DNA
gggcccggagaccagacgagccgggagcccggcaacagggaacccacg

SEQ ID NO: 86 - DNA, 45 bp, synthetic construct
  misc_feature 1..45: note = Synthetic polynucleotide
  source 1..45: mol_type = other DNA
gggcccggagaccagacgagccgggccggcaacagggaacccacg

SEQ ID NO: 87 - DNA, 44 bp, synthetic construct
  misc_feature 1..44: note = Synthetic polynucleotide
  source 1..44: mol_type = other DNA
gggcccggagaccagacgagccgggagcccggcaacagggaacc

SEQ ID NO: 88 - DNA, 17 bp, synthetic construct
  misc_feature 1..17: note = Synthetic polynucleotide
  source 1..17: mol_type = other DNA
agacgagccgggagccc

SEQ ID NO: 89 - DNA, 22 bp, synthetic construct
  misc_feature 1..22: note = Synthetic polynucleotide
  source 1..22: mol_type = other DNA
gccggagccgggaagcccgagc

SEQ ID NO: 90 - DNA, 15 bp, synthetic construct
  misc_feature 1..15: note = Synthetic polynucleotide
  source 1..15: mol_type = other DNA
cggagccaagcccga

SEQ ID NO: 91 - DNA, 30 bp, synthetic construct
  misc_feature 1..30: note = Synthetic polynucleotide
  source 1..30: mol_type = other DNA
ggcgggcgcagcgcaagcgacggacaggcc

SEQ ID NO: 92 - DNA, 28 bp, synthetic construct
  misc_feature 1..28: note = Synthetic polynucleotide
  source 1..28: mol_type = other DNA
ggcggaccgaccggacggagaaacagcc

SEQ ID NO: 93 - protein, 116 aa, synthetic construct
  REGION 1..116: note = Synthetic polypeptide
MAGRSGDSDEELIRTVRLIKLLYQSNPPPNPEGTRQARRNRRRRWRERQRQIHSISERILGTYLGRSAEPVPLQLPPLERLTLDCNEDCGTSGTQGVGSPQILVESPTVLESGTKE

SEQ ID NO: 94 - protein, 17 aa, synthetic construct
  REGION 1..17: note = Synthetic polypeptide
TRQARRNRRRRWRERQR

SEQ ID NO: 95 - protein, 23 aa, synthetic construct
  REGION 1..23: note = Synthetic polypeptide
RDRRRRGSRPSGAERRRRRAAAA

SEQ ID NO: 96 - DNA, 14 bp, synthetic construct
  misc_feature 1..14: note = Synthetic polynucleotide
  source 1..14: mol_type = other DNA
acagaggaacccag

SEQ ID NO: 97 - DNA, 17 bp, synthetic construct
  misc_feature 1..17: note = Synthetic polynucleotide
  source 1..17: mol_type = other DNA
ccggaggacaccacggg

SEQ ID NO: 98 - DNA, 12 bp, synthetic construct
  misc_feature 1..12: note = Synthetic polynucleotide
  source 1..12: mol_type = other DNA
ccacagcacggg

SEQ ID NO: 99 - protein, 129 aa, synthetic construct
  REGION 1..129: note = Synthetic polypeptide
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 100 - protein, 20 aa, synthetic construct
  REGION 1..20: note = Synthetic polypeptide
NAKTRRHERRRKLAIERDTI

SEQ ID NO: 101 - protein, 24 aa, synthetic construct
  REGION 1..24: note = Synthetic polypeptide
GSMDAQTRRRERRAEKQAQWKAAN

SEQ ID NO: 102 - protein, 19 aa, synthetic construct
  REGION 1..19: note = Synthetic polypeptide
GTAKSRYKARRAELIAERR

SEQ ID NO: 103 - protein, 55 aa, synthetic construct
  REGION 1..55: note = Synthetic polypeptide
MQKGNFRNQRKTVKCFNCGKEGHIAKNCRAPRKKGCWKCGKEGHQMKDCTERQAN

SEQ ID NO: 104 - RNA, 15 nt, synthetic construct
  misc_feature 1..15: note = Synthetic polynucleotide
  source 1..15: mol_type = other RNA
gcgctgacaaagcgc

SEQ ID NO: 105 - RNA, 19 nt, synthetic construct
  misc_feature 1..19: note = Synthetic polynucleotide
  source 1..19: mol_type = other RNA
gggccctgaagaagggccc

SEQ ID NO: 106 - RNA, 20 nt, synthetic construct
  misc_feature 1..20: note = Synthetic polynucleotide
  source 1..20: mol_type = other RNA
tctcaacctaaccgttgaga

SEQ ID NO: 107 - RNA, 20 nt, synthetic construct
  misc_feature 1..20: note = Synthetic polynucleotide
  source 1..20: mol_type = other RNA
ggactagcggaggctagtcc

SEQ ID NO: 108 - protein, 14 aa, synthetic construct
  REGION 1..14: note = Synthetic polypeptide
SGFANELGPRLMGH

SEQ ID NO: 109 - protein, 8 aa, synthetic construct
  REGION 1..8: note = Synthetic polypeptide
PPKKKRKV

SEQ ID NO: 110 - protein, 29 aa, synthetic construct
  REGION 1..29: note = Synthetic polypeptide
MMSFVSLLLVGILFWATEAEQLTKCEVFQ

SEQ ID NO: 111 - protein, 4 aa, synthetic construct
  REGION 1..4: note = Synthetic polypeptide
KDEL

SEQ ID NO: 112 - protein, 25 aa, synthetic construct
  REGION 1..25: note = Synthetic polypeptide
MLSLRQSIRFFLPATRTLCSSRYLL

SEQ ID NO: 113 - protein, 9 aa, synthetic construct
  REGION 1..9: note = Synthetic polypeptide
  REGION 3..7: note = misc_feature - Xaa can be any naturally occurring amino acid
RLXXXXXHL

SEQ ID NO: 114 - protein, 7 aa, synthetic construct
  REGION 1..7: note = Synthetic polypeptide
PKKKRKV

SEQ ID NO: 115 - protein, 30 aa, synthetic construct
  REGION 1..30: note = Synthetic polypeptide
MDSLLMNRRKFLYQFKNVRWAKGRRETYLC

SEQ ID NO: 116 - RNA, 57 nt, synthetic construct
  misc_feature 1..57: note = Synthetic polynucleotide
  source 1..57: mol_type = other RNA
ccaagggatcaatcggtctctcgagggtccgagtctagaccagattggtctctctgg

(19) United States
(12) Patent Application Publication
(10) Pub. No.: US 2024/0082420 A1
(43) Pub. Date: Mar. 14, 2024

(54) DELIVERY OF THERAPEUTIC RNAS VIA ARRDC1-MEDIATED MICROVESICLES

(71) Applicant: President and Fellows of Harvard College, Cambridge, MA (US)

(72) Inventor: Quan Lu, Cambridge, MA (US)

(73) Assignee: President and Fellows of Harvard College, Cambridge, MA (US)

(21) Appl. No.: 18/343,685

(22) Filed: Jun. 28, 2023

Related U.S. Application Data
(63) Continuation of application No. 16/338,969, filed on Apr. 2, 2019, now Pat. No. 11,730,823.
(60) Provisional application No. 62/403,678, filed on Oct. 3, 2016.

(51) Int. Cl. A61K 47/69; A61K 9/50; A61K 47/54; A61K 47/62; C12N 15/87

(52) U.S. Cl. CPC A61K 47/6901; A61K 9/5068; A61K 47/54; A61K 47/62; C12N 15/87; C12N 2740/16322; C12N 2795/10222; C12N 2795/10322; C12N 2795/18122

(57) ABSTRACT

Methods, systems, compositions and strategies for the delivery of RNA into cells in vivo, ex vivo, or in vitro via ARMMs are provided. In some aspects, ARMMs containing fusion proteins of ARRDC1 fused to an RNA binding protein or an RNA binding protein fused to a WW domain are provided. In some aspects, ARMMs containing binding RNAs associated with cargo RNAs are provided. In other aspects, cargo RNAs associated with a binding RNA, such as a TAR element, are loaded into ARMMs via ARRDC1 fusion proteins containing an RNA binding protein, such as trans-activator of transcription (Tat) protein.

### RELATED APPLICATIONS

This application is a national stage filing under 35 U.S.C. § 371 of international PCT application, PCT/US2017/054912, filed Oct. 3, 2017, which claims priority under 35 U.S.C. § 119(e) to U.S. Provisional Application, U.S. Ser. No. 62/403,678, filed on Oct. 3, 2016, each of which is incorporated herein by reference.

### GOVERNMENT SUPPORT

This invention was made with government support under HL 114769 awarded by National Institutes of Health. The government has certain rights in the invention.

### BACKGROUND OF THE INVENTION

The delivery of ribonucleic acids (e.g., therapeutic RNAs) to cells is limited by a number of factors, including the immunogenicity of viral delivery systems as well as the ability to a target a specific cell type when using viral or non-viral transduction methods. Therefore, there is a need to develop methods, compositions, and systems for effectively delivering therapeutic RNAs, such as mRNAs or siRNAs, to a desired targeted cell in order to realize the full potential of RNA-based therapeutics.

### SUMMARY OF THE INVENTION

This invention relates to the discovery that ribonucleic acids (RNAs) can be loaded into microvesicles, specifically ARRDC1-mediated microvesicles (ARMMs), for delivery to a targeted cell. The ARMM delivery system, described herein, addresses many limitations of current delivery systems that prevent the safe and efficient delivery of therapeutic RNAs to cells. As ARMMS are derived from an endogenous budding pathway, they are unlikely to elicit a strong immune response, unlike viral delivery systems, which are known to trigger an inflammatory response (Sen et al., “Cellular unfolded protein response against viruses used in gene therapy.” Front Microbiology. 2014; 5:250, 1-16.). Additionally, ARMMs allow for the specific packaging of any cargo RNA of interest (e.g., a mRNA or a siRNA). These cargo RNAs can then be delivered by fusion with or uptake by specific recipient cells/tissues by incorporating antibodies or other types of molecules in the ARMMs that recognize tissue-specific markers. ARMMs are microvesicles that are distinct from exosomes and, like budding viruses, are produced by direct plasma membrane budding (DPMB). DPMB is driven by a specific interaction of TSG101 with a tetrapeptide PSAP (SEQ ID NO: 1) motif of the arrestin-domain-containing protein ARRDC1 accessory protein, which is localized to the plasma membrane through its arrestin domain. ARMMS have been described in detail, for example, in PCT application number PCT/US2013/024839, filed Feb. 6, 2013 (published as WO 2013/119602 A1 on Aug. 15, 2013) by Lu et al., and entitled “Arrdc1-Mediated Microvesicles (ARMMs) and Uses Thereof,” the entire contents of which are incorporated herein by reference. The ARRDC1/TSG101 interaction results in relocation of TSG101 from endosomes to the plasma membrane and mediates the release of microvesicles that contain TSG101, ARRDC1, and other cellular components as well as the cargo RNA of interest.

Non-naturally occurring RNAs including, for example, a binding RNA (e.g., a TAR element) associated with a cargo RNA (e.g., an RNA that expresses GFP, p53, Bims, or other protein) can associate with one or more ARMM proteins (e.g., ARRDC1), facilitating their incorporation into ARMMs, which in turn can be used to deliver the cargo RNA into a targeted cell. As one example, a cargo RNA fused to a TAR element can associate with an ARRDC1 protein that is fused to an RNA binding protein, such as a Tat protein. A non-limiting example of an ARRDC1 protein fused to a Tat protein is shown in FIG. 1, Panel A. As another example, a cargo RNA fused to a TAR element can associate with a WW domain-containing protein that is fused to an RNA binding protein, such as a Tat protein. The WW domain-containing protein that is fused to the RNA binding protein (e.g., Tat protein) can associate with ARRDC1, for example, by binding to the PPXY (SEQ ID NO: 2) motif of ARRDC1. A non-limiting example of a Tat protein fused to a WW domain that associates with the PPXY (SEQ ID NO: 2) motif of ARRDC1 is shown in FIG. 1, Panel B. The association of a cargo RNA to an ARMM protein (e.g., ARRDC1), for example, via the Tat/TAR interaction, facilitates loading of the cargo RNA into the ARRDC1-containing ARMM. For example, a cargo RNA fused to a TAR element may associate with an ARRDC1 protein fused to a Tat protein via the association between Tat and TAR, as illustrated in FIG. 2. As another example a cargo RNA fused to a TAR element may associate with a Tat protein that is fused to a WW domain, which may associate with an ARRDC1 protein via the association between the WW domain and the PPXY (SEQ ID NO: 2) motif of the ARRDC1 protein. In certain instances, the cargo RNA can be fused to or associated with a binding RNA via a linker, which may be cleaved upon delivery into a target cell. The binding RNA (e.g. TAR element) and the RNA binding protein (e.g., Tat protein) may be any suitable RNA and protein pair that sufficiently associates to facilitate loading of a cargo RNA into an ARMM.

Other advantages, features, and uses of the invention will be apparent from the detailed description of certain exemplary, non-limiting embodiments; the drawings; the non-limiting working examples; and the claims.

### BRIEF DESCRIPTION OF THE DRAWINGS

FIG. 1 shows non-limiting schematic representations of fusion proteins used for packaging RNAs into ARMMs. (A) is a schematic of an ARRDC1 protein, containing a PPXY (SEQ ID NO: 2) motif, that is fused to a Tat protein. (B) is a schematic of a WW domain fused to a Tat protein, which may bind the PPXY (SEQ ID NO: 2) motif of ARRDC1 via the interaction between the WW domain and the PPXY (SEQ ID NO: 2) motif.

FIG. 2 shows a non-limiting schematic representation of an ARRDC1 protein fused to a Tat protein that associates with a TAR molecule that is fused to a cargo RNA. The nucleotide sequence of a TAR is set forth in SEQ ID NO: 116.

FIG. 3 is a non-limiting schematic of a ubiquitin ligase protein (top) showing the conserved protein domains including the phospholipid binding C2 domain, four WW domains that bind PPXY (SEQ ID NO: 2) motifs, and the HECT ubiquitin ligase domain. Exemplary ubiquitin ligases (bottom) include Nedd4-1, Nedd4-2, WWP1, WWP2, Smurf1, Smurf2, ITCH, NEDL1, and NEDL2.

FIG. 4 is a schematic demonstrating the production of an ARMM in a microvesicle-producing cell (ARMM producing cell) that contains an ARRDC1-Tat fusion protein, which associates with a TAR molecule fused to a cargo RNA to facilitate the loading of the cargo RNA into the ARMM. The ARRDC1-Tat fusion protein may be co-expressed in an ARMM producing cell with the TAR:cargoRNA fusion (e.g., from a plasmid DNA) so they are co-incorporated into ARMMs (left). The ARMM may then be delivered to an ARMM target cell (right), where the cargo RNA fused to the TAR is released into the cytoplasm of the target cell. The cargo RNA may then be translated into protein, for example, if the RNA is an mRNA. Alternatively, the cargo RNA may be a siRNA, which may be processed by a Dicer complex to stimulate the RNA interference (RNAi) pathway.

FIG. 5 provides a Western blots showing that an ARRDC1-Tat fusion protein maintains the ability to bud out of cells as ARRDC1-containing ARMMs. For example, cells expressing either the ARRDC1-Tat fusion protein or the ARRDC1 tagged with an OLLAS epitope tag (ARRDC1-OLLAS), which lacks the Tat peptide, produced ARMMs containing ARRDC1-Tat or ARRDC1-OLLAS, respectively. The Western blots further show that plasmid DNA encoding GFP alone or TAR fused to GFP (TAR-GFP) were both capable of expressing GFP protein in cells transfected with the plasmid DNA. The OLLAS epitope tag comprises the amino acid sequence SGFANELGPRLMGH (SEQ ID NO: 108)

FIG. 6 is a graph showing that TAR-GFP mRNA was more efficiently packaged into ARMMs using the Tat/TAR system. The relative amount of GFP mRNA detected in ARMMs as compared to their respective ARMM producing cells was significantly increased when ARRDC1-Tat and TAR:GFP were co-expressed in cells as compared to cells that co-expressed ARRDC1-OLLAS and GFP; ARRDC1-OLLAS and TAR-GFP; or ARRDC1-Tat and GFP ARRDC1-OLLAS.

FIG. 7 are graphs showing the relative levels of hypoxanthine-guanine phosphoribosyltransferase (HPRT) control mRNA in (A) ARMM producing cells that express combinations of GFP and ARRDC1-Tat; GFP and ARRDC1-OLLAS; TAR-GFP and ARRDC1-Tat; TAR-GFP and ARRDC1-OLLAS; or a control that does not express any of the constructs, and (B) ARMMs from the ARMM producing cells of (A).

FIG. 8 is a graph showing that TAR-GFP mRNA was efficiently packaged into ARMMs in a dose-dependent manner. The relative amount of GFP mRNA detected in ARMMs as compared to their respective ARMM producing cells increased in a dose-dependent manner for cells co-expressing TAR-GFP and ARRDC1-Tat but not in cells co-expressing GFP and ARRDC1-Tat. The amounts of GFP or TAR-GFP transfected into cells was 500 ng, 50 ng, and 5 ng, respectively.

FIG. 9 are graphs showing the relative levels of hypoxanthine-guanine phosphoribosyltransferase (HPRT) control mRNA in (A) ARMM producing cells that were transfected with 500 ng, 50 ng, or 5 ng of either GFP or TAR-GFP, respectively, and (B) ARMMs from the ARMM producing cells of (A).

FIG. 10 are graphs showing that ARMMs containing TAR-GFP mRNA were capable of delivering the TAR-GFP mRNA to a target cells in vitro. (A) The relative amount of GFP mRNA delivered to recipient cells was greater when using ARMMs containing ARRDC1-Tat and TAR-GFP as compared to ARMMs containing ARRDC1-Tat and GFP alone. The relative levels of hypoxanthine-guanine phosphoribosyltransferase (HPRT) control mRNA are shown for recipient cells in (B) and for donor ARMM producing cells in (C). The relative amount of GFP mRNA in ARMMs was greater in ARMMs produced from donor cells expressing ARRDC1-Tat and TAR-GFP as compared to ARMMs produced from donor cells expressing ARRDC1-Tat and GFP alone (D). The relative levels of HPRT control mRNA in ARMMs produced from donor cells expressing ARRDC1-Tat and TAR-GFP, or ARRDC1-Tat and GFP are shown in (E).

FIG. 11 shows packaging and delivery of RNAs via ARMMs. (A) Shows a schematic of an RNA packaging strategy. Tat peptide, which binds specifically to TAR, is fused to the C-terminus of ARRDC1 to recruit RNA cargo molecules linked to TAR, into ARMMs. (B) Shows packaging of TAR-GFP mRNA in ARMMs. ARRDC1-Tat was co-transfected with TAR-GFP or control GFP construct into HEK293T cells. ARMMs were pelleted via ultracentrifugation. qRT-PCR was done on ARMMs and on the transfected cells for GFP and for a control mRNA (HPRT1). (C) Shows packaging of TAR-p53 mRNA in ARMMs. TAR-p53 was co-transfected with ARRDC1 or ARRDC1-Tat construct into HEK293T cells. ARMMs were pelleted via ultracentrifugation. qRT-PCR was done on ARMMs and on the transfected cells for TAR-p53 and for HPRT1. (D) Shows Transfer of TAR-GFP mRNA into recipient cells. A549 cells were incubated with ARMMs containing TAR-GFP mRNA overnight, washed with PBS extensively and subjected to mRNA analysis by qRT-PCR. (E) Shows transfer of TAR-GFP mRNA into recipient cells. p53-null H1299 cells were incubated with ARMMs containing TAR-p53 mRNA overnight, washed with PBS extensively and subjected to mRNA analysis by qRT-PCR. (F) Shows translation of ARMMs-delivered GFP mRNA in recipient cells. A549 cells were incubated with ARMMs containing TAR-GFP mRNA for 24 h with or without the translational inhibitor cycloheximide (CHX), and subjected to flow cytometry analysis. (G) Shows activation of p53 target genes in recipient cells receiving TAR-p53 ARMMs. P53-null H1299 cells were incubated with ARMMs containing TAR-p53 mRNA for 18 h and subjected to mRNA analysis by qRT-PCR to detect MDM2 and p21 mRNAs. At least 3 independent replicates were done for all assays. * p<0.05; ** p<0.01.

FIG. 12 shows (A) GFP or TAR-GFP was co-transfected with ARRDC1-Tat into HEK293T cells. (B) ARRDC1 or ARRDC1-Tat was co-transfected with TAR-p53 into HEK293T cells. Medium was collected for extracellular vesicles. Cell lysates and vesicles were subjected to Western blot analysis using indicated antibodies.

### DEFINITIONS

The term “ARMM,” as used herein, refers to a microvesicle comprising an ARRDC1 protein or variant thereof, and/or TSG101 protein or variant thereof. In some embodiments, the ARMM is shed from a cell, and comprises a molecule, for example, a nucleic acid, protein, or small molecule, present in the cytoplasm or associated with the membrane of the cell. In some embodiments, the ARMM is shed from a transgenic cell comprising a recombinant expression construct that includes the transgene, and the ARMM comprises a gene product, for example, a transcript and/or a protein (e.g., an ARRDC1-Tat fusion protein and a TAR-cargo RNA) encoded by the expression construct. In some embodiments, the protein encoded by the expression construct is a Tat protein fused to at least one WW domain, or variant thereof, which may associate with the ARRDC1 protein to facilitate loading of cargo RNA fused to a TAR into the ARMM. In some embodiments, the ARMM is produced synthetically, for example, by contacting a lipid bilayer within ARRDC1 protein, or variant thereof, in a cell-free system in the presence of TSG101, or a variant thereof. In other embodiments, the ARMM is synthetically produced by further contacting a lipid bilayer with HECT domain ligase, and VPS4a. In some embodiments, an ARMM lacks a late endosomal marker. Some ARMMs as provided herein do not include, or are negative for, one or more exosomal biomarker. Exosomal biomarkers are known to those of skill in the art and include, but are not limited to, CD63, Lamp-1, Lamp-2, CD9, HSPA8, GAPDH, CD81, SDCBP, PDCD6IP, ENO1, ANXA2, ACTB, YWHAZ, HSP90AAI, ANXA5, EEF1A1, YWHAE, PPIA, MSN, CFL1, ALDOA, PGK1, EEF2, ANXA1, PKM2, HLA-DRA, and YWHAB. For example, some ARMMs provided herein lack CD63, some ARMMs lack LAMP1, some ARMMs lack CD9, some ARMMs lack CD81, some ARMMs lack CD63 and Lamp-1, some ARMMs lack CD63, Lamp-1, and CD9, some ARMMs lack CD63, Lamp-1, CD81, and CD9, and so forth. Certain ARMMs provided herein may include an exosomal biomarker. Accordingly, some ARMMs may be negative for one or more exosomal biomarker, but positive for one or more different exosomal biomarker. For example, such an ARMM may be negative for CD63 and Lamp-1, but may include PGK1 or GAPDH; or may be negative for CD63, Lamp-1, CD9, and CD81, but may be positive for HLA-DRA. In some embodiments, ARMMs include an exosomal biomarker, but at a lower level than a level found in exosomes. For example, some ARMMs include one or more exosomal biomarkers at a level of less than about 1%, less than about 5%, less than about 10%, less than about 20%, less than about 30%, less than about 40%, or less than about 50% of the level of that biomarker found in exosomes. To give a non-limiting example, in some embodiments, an ARMM may be negative for CD63 and Lamp-1, include CD9 at a level of less than about 5% of the level of CD9 typically found in exosomes, and be positive for ACTB. Exosomal biomarkers in addition to those listed above are known to those of skill in the art, and the invention is not limited in this regard.

The term “binding RNA”, as used herein, refers to a ribonucleic acid (RNA) that binds to an RNA binding protein, for example, any of the RNA binding proteins known in the art and/or provided herein. In some embodiments, a binding RNA is an RNA that specifically binds to an RNA binding protein. A binding RNA that “specifically binds” to an RNA binding protein, binds to the RNA binding protein with greater affinity, avidity, more readily, and/or with greater duration than it binds to another protein, such as a protein that does not bind the RNA or a protein that weakly binds to the binding RNA. In some embodiments, the binding RNA is a naturally-occurring RNA, or non-naturally-occurring variant thereof, that binds to a specific RNA binding protein. For example, the binding RNA may be a TAR element, a Rev response element, an MS2 RNA, or any variant thereof that specifically binds an RNA binding protein. In some embodiments, the binding RNA may be a trans-activating response element (TAR element), or variant thereof, which is an RNA stem-loop structure that is found at the 5′ ends of nascent HIV-1 transcripts and specifically binds to the trans-activator of transcription (Tat) protein. In some embodiments, the binding RNA is a Rev response element (RRE), or variant thereof, that specifically binds to the accessory protein Rev (e.g., Rev from HIV-1). In some embodiments, the binding RNA is an MS2 RNA that specifically binds to a MS2 phage coat protein. The binding RNAs of the present disclosure may be designed to specifically bind a protein (e.g., an RNA binding protein fused to ARRDC1) in order to facilitate loading of the binding RNA (e.g., a binding RNA fused to a cargo RNA) into an ARMM.

The term “aptamer”, as used herein, refers to nucleic acids that bind to a specific target molecule, e.g., an RNA binding protein. In some embodiments, nucleic acid (e.g., DNA or RNA) aptamers are engineered through repeated rounds of in vitro selection or equivalently, SELEX (systematic evolution of ligands by exponential enrichment) methodology to bind to various molecular targets, for example, proteins, small molecules, macromolecules, metabolites, carbohydrates, metals, nucleic acids, cells, tissues, and organisms. Methods for engineering aptamers to bind to various molecular targets, such as proteins, are known in the art and include those described in U.S. Pat. Nos. 6,376,19; and 9,061,043; Shui B., et al., “RNA aptamers that functionally interact with green fluorescent protein and its derivatives.” Nucleic Acids Res., March; 40(5): e39 (2012); Trujillo U. H., et al., “DNA and RNA aptamers: from tools for basic research towards therapeutic applications”. Comb Chem High Throughput Screen 9 (8): 619-32 (2006); Srisawat C., et al., “Streptavidin aptamers: Affinity tags for the study of RNAs and ribonucleoproteins.” RNA, 7:632-641 (2001); and Tuerk and Gold, “Systematic evolution of ligands by exponential enrichment: RNA ligands to bacteriophage T4 DNA polymerase.” Science. 1990; the entire contents of each of which are hereby incorporated by reference in their entirety.

The term “RNA binding protein”, as used herein refers to a polypeptide molecule that binds to a binding RNA, for example, any of the binding RNAs known in the art and/or provided herein. In some embodiments, an RNA binding protein is a protein that specifically binds to a binding RNA. An RNA binding protein that “specifically binds” to a binding RNA, binds to the binding RNA with greater affinity, avidity, more readily, and/or with greater duration than it binds to another RNA, such as a control RNA (e.g., an RNA having a random nucleic acid sequence) or an RNA that weakly binds to the RNA binding protein. In some embodiments, the RNA binding protein is a naturally-occurring protein, or non-naturally-occurring variant thereof, that binds to a specific RNA. For example, in some embodiments, the RNA binding protein may be a trans-activator of transcription (Tat) protein that specifically binds a trans-activating response element (TAR element). In some embodiments, the RNA binding protein is a regulator of virion expression (Rev) protein (e.g., Rev from HIV-1) or variant thereof, that specifically binds to a Rev response element (RRE). In some embodiments, the RNA binding protein is a coat protein of an MS2 bacteriophage that specifically binds to an MS2 RNA. The RNA binding proteins useful in the present disclosure (e.g., a binding protein fused to ARRDC1) may be designed to specifically bind a binding RNA (e.g., a binding RNA fused to a cargo RNA) in order to facilitate loading of the binding RNA into an ARMM.

The term “cargo RNA”, as used herein, refers to a ribonucleic acid that may be incorporated into an ARMM, for example, into the liquid phase of the ARMM (e.g., by associating the cargo RNA with an RNA binding protein fused to an ARRDC1 protein). The term “cargo RNA to be delivered” refers to any RNA that can be delivered via its association with or inclusion in an ARMM to a subject, organ, tissue, or cell. In some embodiments, the cargo RNA is to be delivered to a targeted cell in vitro, in vivo, or ex vivo. In some embodiments, the cargo RNA to be delivered is a biologically active agent, i.e., it has activity in a cell, organ, tissue, and/or subject. For instance, an RNA that, when administered to a subject, has a biological effect on that subject or is considered to be biologically active. In certain embodiments, the cargo RNA is a messenger RNA or an RNA that expresses a protein in a cell. In certain embodiments, the cargo RNA is a small interfering RNA (siRNA) that inhibits the expression of one or more genes in a cell. In some embodiments, a cargo RNA to be delivered is a therapeutic agent. As used herein, the term “therapeutic agent” refers to any agent that, when administered to a subject, has a beneficial effect. In some embodiments, the cargo RNA to be delivered to a cell is an RNA that expresses a transcription factor, a tumor suppressor, a developmental regulator, a growth factor, a metastasis suppressor, a pro-apoptotic protein, a nuclease, or a recombinase. In certain embodiments, the cargo RNA is associated with a binding RNA, either covalently or non-covalently (e.g., via nucleotide base pairing) to facilitate loading of the cargo RNA into an ARMM.

The term “linker,” as used herein, refers to a chemical moiety linking two molecules or moieties, e.g., an ARRDC1 protein and a Tat protein, or a WW domain and a Tat protein. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, group, polymer, or other chemical moiety. In some embodiments, the linker is a cleavable linker, e.g., the linker comprises a bond that can be cleaved upon exposure to, for example, UV light or a hydrolytic enzyme, such as a lysosomal protease. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond).

As used herein, the term “animal” refers to any member of the animal kingdom. In some embodiments, the term “animal” refers to a human of either sex at any stage of development. In some embodiments, the term “animal” refers to a non-human animal at any stage of development. In certain embodiments, the non-human animal is a mammal (e.g., a rodent, a mouse, a rat, a rabbit, a monkey, a dog, a cat, a sheep, cattle, a primate, or a pig). Animals include, but are not limited to, mammals, birds, reptiles, amphibians, fish, and worms. In some embodiments, the animal is a transgenic animal, genetically-engineered animal, or a clone. In some embodiments, the animal is a transgenic non-human animal, genetically-engineered non-human animal, or a non-human clone.

As used herein, the term “associated with,” when used with respect to two or more entities, for example, with chemical moieties, molecules, and/or ARMMs, means that the entities are physically associated or connected with one another, either directly or via one or more additional moieties that serves as a linker, to form a structure that is sufficiently stable so that the entities remain physically associated under the conditions in which the structure is used, e.g., physiological conditions. An ARMM is typically associated with an agent, for example, a nucleic acid, protein, or small molecule, by a mechanism that involves a covalent (e.g., via an amide bond) or non-covalent association (e.g., between ARRDC1 and a WW domain, or between a Tat protein and a TAR element). In certain embodiments, the agent to be delivered (e.g., a cargo RNA) is covalently bound to a molecule (e.g., a TAR element) that associates non-covalently with a part of the ARMM, for example, a Tat protein, or variant thereof, that is fused to an ARRCD1 protein, or variant thereof. In some embodiments, the agent to be delivered (e.g., a cargo RNA) is covalently bound to a molecule (e.g., a TAR element) that associates non-covalently with a Tat protein, or variant thereof, that is fused to a WW domain, where the WW domain non-covalently associates with ARRDC1 in an ARMM. In some embodiments, the association is via a linker, for example, a cleavable linker. In some embodiments, an entity (e.g., a cargo RNA) is associated with an ARMM by inclusion in the ARMM, for example, by encapsulation of an entity (e.g., a cargo RNA) within the ARMM. For example, in some embodiments, an agent (e.g., a cargo RNA) present in the cytoplasm of an ARMM-producing cell is associated with an ARMM by encapsulation of the cytoplasm with the agent in the ARMM upon ARMM budding. Similarly, a membrane protein or other molecule associated with the cell membrane of an ARMM producing cell may be associated with an ARMM produced by the cell by inclusion into the ARMM's membrane upon budding.

As used herein, the phrase “biologically active” refers to a characteristic of any substance that has activity in a cell, organ, tissue, and/or subject. For instance, a substance that, when administered to an organism, has a biological effect on that organism, is considered to be biologically active. As one example, a cargo RNA may be considered biologically active if it increases or decreases the expression of a gene product when administered to a subject or cell.

As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95% identical, about 98% identical, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

The term “engineered,” as used herein refers to a protein, nucleic acid, complex, substance, or entity that has been designed, produced, prepared, synthesized, and/or manufactured by a human. Accordingly, an engineered product is a product that does not occur in nature. In some embodiments, an engineered protein or nucleic acid is a protein or nucleic acid that has been designed to meet particular requirements or to have particular design features. For example, a cargo RNA may be engineered to associate with the ARRDC1 by fusing one or more WW domains to a Tat protein and fusing the cargo RNA to a TAR element to facilitate loading of the cargo RNA into an ARMM. As another example, a cargo RNA may be engineered to associate with the ARRDC1 by fusing a Tat protein to the ARRDC1 and by fusing the cargo RNA to a TAR element to facilitate loading of the cargo RNA into an ARMM.

As used herein, “expression” of a nucleic acid sequence refers to one or more of the following events: (1) production of an RNA transcript from a DNA sequence (e.g., by transcription); (2) processing of an RNA transcript (e.g., by splicing, editing, 5′ cap formation, and/or 3′ end processing); (3) translation of an RNA transcript into a polypeptide or protein; and (4) post-translational modification of a polypeptide or protein.

As used herein, a “fusion protein” includes a first protein moiety, e.g., an ARRCD1 protein or variant thereof, associated with a second protein moiety, for example, a Tat protein to be delivered to a target cell through a peptide linkage. In certain embodiments, the fusion protein is encoded by a single fusion gene.

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that the definition of gene includes references to nucleic acids that do not encode proteins but rather encode functional RNA molecules, such as gRNAs, RNAi agents, ribozymes, tRNAs, etc. For the purpose of clarity it should be noted that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used herein refers to a protein-coding nucleic acid.

As used herein, the term “gene product” or “expression product” generally refers to an RNA transcribed from the gene (pre- and/or post-processing) or a polypeptide (pre- and/or post-modification) encoded by an RNA transcribed from the gene.

As used herein, the term “green fluorescent protein” (GFP) refers to a protein originally isolated from the jellyfish Aequorea victoria that fluoresces green when exposed to blue light or a derivative of such a protein (e.g., an enhanced or wavelength-shifted version of the protein). The amino acid sequence of wild type GFP is as follows:

(SEQ ID NO: 35)
MSKGEELFTGVVPILVELDGDVNGHKFSVSGEGEGDATYGKLTLKFICTT
GKLPVPWPTLVTTFSYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHY
LSTQSALSKDPNEKRDHMVLLEFVTAAGITHGMDELYK

Proteins that are at least 70%, at least 75%, at least 80%, at least 85%, at least 90%, at least 95%, at least 98%, or at least 99% homologous to SEQ ID NO: 35 are also considered to be green fluorescent proteins.

As used herein, the term “homology” refers to the overall relatedness between nucleic acids (e.g. DNA molecules and/or RNA molecules) or polypeptides. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% identical. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotide sequences or amino acid sequences). In accordance with the invention, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for sequences to be considered homologous. For nucleotide sequences less than 60 nucleotides in length, homology is determined by the ability to encode a stretch of at least 4-5 uniquely specified amino acids. In accordance with the invention, two protein sequences are considered to be homologous if the proteins are at least about 50% identical, at least about 60% identical, at least about 70% identical, at least about 80% identical, or at least about 90% identical for at least one stretch of at least about 20 amino acids.

As used herein, the term “identity” refers to the overall relatedness between nucleic acids or proteins (e.g. DNA molecules, RNA molecules, and/or polypeptides). Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, the term “nucleic acid,” in its broadest sense, refers to a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleotides. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g. polynucleotides and residues, including nucleotides and/or nucleosides) that have not been chemically modified in order to facilitate or achieve delivery.

As used herein, the term “protein” refers to a string of at least two amino acids linked to one another by one or more peptide bonds. Proteins may include moieties other than amino acids (e.g., may be glycoproteins) and/or may be otherwise processed or modified. Those of ordinary skill in the art will appreciate that a “protein” can be a complete protein chain as produced by a cell (with or without a signal sequence), or can be a functional portion thereof. Those of ordinary skill will further appreciate that a protein can sometimes include more than one protein chain, for example linked by one or more disulfide bonds or associated by other means. Proteins may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In certain embodiments, the modifications of the protein lead to a more stable protein (e.g., greater half-life in vivo). These modifications may include cyclization of the protein, the incorporation of D-amino acids, etc. None of the modifications should substantially interfere with the desired biological activity of the protein. In certain embodiments, the modifications of the protein lead to a more biologically active protein. In some embodiments, proteins may comprise natural amino acids, non-natural amino acids, synthetic amino acids, amino acid analogs, and combinations thereof.

As used herein, the term “reprogramming factor” refers to a factor that, alone or in combination with other factors, can change the state of a cell from a somatic, differentiated state into a pluripotent stem cell state. Non-limiting examples of reprogramming factors include a protein (e.g., a transcription factor), a peptide, a nucleic acid, or a small molecule. Known reprogramming factors that are useful for cell reprogramming include, but are not limited to, Oct4, Sox2, Klf4, and c-myc. Similarly, a programming factor may be used to modulate cell differentiation, for example, to facilitate or induce cell differentiation towards a desired lineage.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals, such as mice, rats, rabbits, non-human primates, and humans) and/or plants. In some embodiments, the subject is a patient having or suspected of having a disease or disorder. In other embodiments, the subject is a healthy volunteer.

As used herein, the term “therapeutically effective amount” means an amount of an agent to be delivered (e.g., nucleic acid, protein, drug, therapeutic agent, diagnostic agent, prophylactic agent, RNA, ARMM, or ARMM comprising a cargo RNA) that is sufficient, when administered to a subject suffering from or susceptible to a disease, disorder, and/or condition, to treat, improve symptoms of, diagnose, prevent, and/or delay the onset of the disease, disorder, and/or condition.

As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules. Examples of transcription factors include, but are not limited to, Sp1, NF1, CCAAT, GATA, HNF, PIT-1, MyoD, Myf5, Hox, Winged Helix, SREBP, p53, CREB, AP-1, Mef2, STAT, R-SMAD, NF-uB, Notch, TUBBY, and NFAT.

As used herein, the term “treating” refers to partially or completely preventing, and/or reducing the incidence of one or more symptoms or features of a particular disease or condition. For example, “treating” cancer may refer to inhibiting survival, growth, and/or spread of the cancer. Treatment may be administered to a subject who does not exhibit signs or symptoms of a disease, disorder, and/or condition and/or to a subject who exhibits only early signs or symptoms of a disease, or condition for the purpose of decreasing the risk of developing more severe effects associated with the disease, disorder, or condition.

As used herein, “vector” refers to a nucleic acid molecule which can transport another nucleic acid to which it has been linked. In some embodiment, vectors can achieve extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

The term “WW domain” as used herein, refers to a protein domain having two basic residues at the C-terminus that mediates protein-protein interactions with short proline-rich or proline-containing motifs. It should be appreciated that the two basic residues (e.g., H, R, and K) of the WW domain are not required to be at the absolute C-terminal end of the WW protein domain. Rather, the two basic residues may be at a C-terminal portion of the WW protein domain (e.g., the C-terminal half of the WW protein domain). In some embodiments, the WW domain contains at least 1, 2, 3, 4, 5, 6, 7, 8, 9, or 10 W residues. In some embodiments, the WW domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW domain possessing the two basic C-terminal amino acid residues may have the ability to associate with short proline-rich or proline-containing motifs (e.g., a PPXY (SEQ ID NO: 2) motif). WW domains bind a variety of distinct peptide ligands including motifs with core proline-rich sequences, such as PPXY (SEQ ID NO: 2), which is found in AARDC1. A WW domain may be a 30-40 amino acid protein interaction domain with two signature tryptophan residues spaced by 20-22 amino acids. The three-dimensional structure of WW domains shows that they generally fold into a three-stranded, antiparallel p sheet with two ligand-binding grooves.

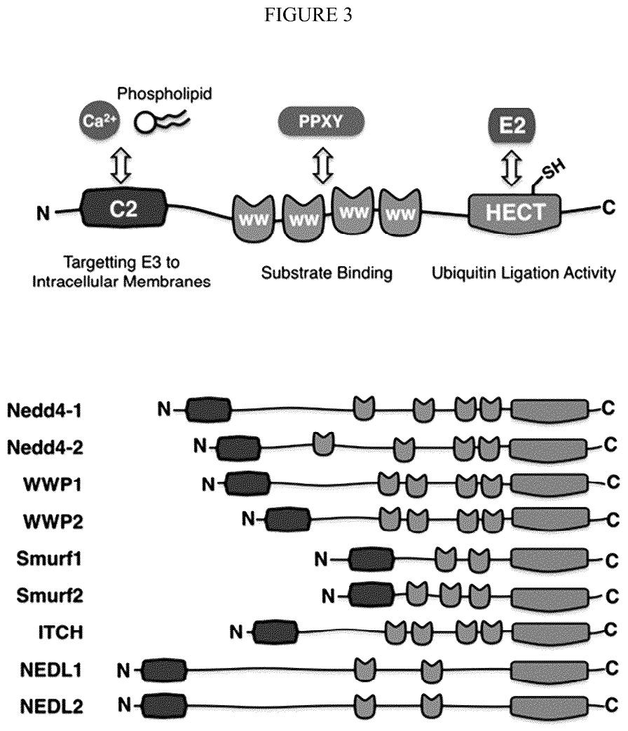

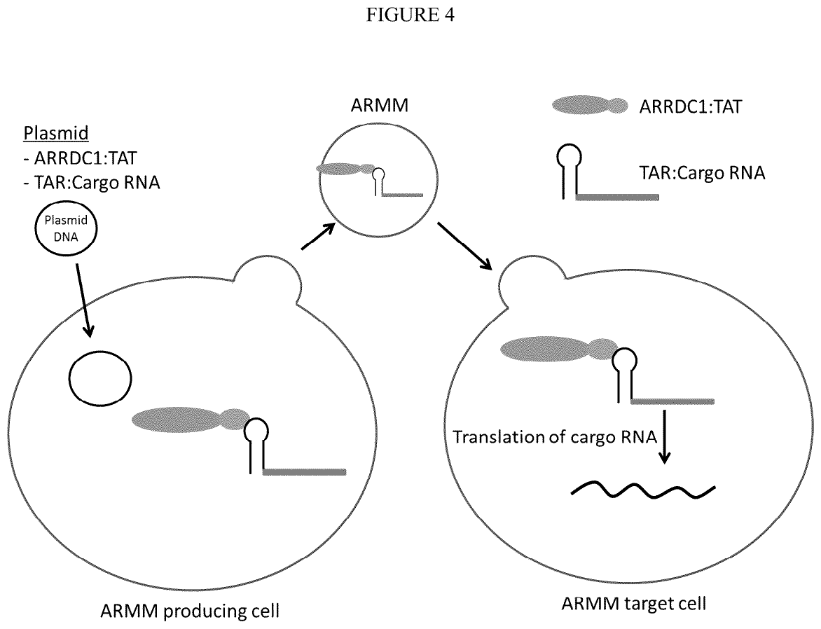

WW domains are found in many eukaryotes and are present in approximately 50 human proteins (Bork, P. & Sudol, M. The WW domain: a signaling site in dystrophin? Trends Biochem Sci 19, 531-533 (1994)). WW domains may be present together with several other interaction domains, including membrane targeting domains, such as C2 in the NEDD4 family proteins, the phosphotyrosine-binding (PTB) domain in FE65 protein, FF domains in CA150 and FBPII, and pleckstrin homology (PH) domains in PLEKHA5. WW domains are also linked to a variety of catalytic domains, including HECT E3 protein-ubiquitin ligase domains in NEDD4 family proteins, rotomerase or peptidyl prolyisomerase domains in Pin1, and Rho GAP domains in ArhGAP9 and ArhGAP12. Exemplary proteins containing WW domains are illustrated in FIG. 3.

In the instant disclosure, the WW domain may be a WW domain that naturally possesses two basic amino acids at the C-terminus. In some embodiments, a WW domain or WW domain variant may be from the human ubiquitin ligase WWP1, WWP2, Nedd4-1, Nedd4-2, Smurf1, Smurf2, ITCH, NEDL1, or NEDL2. Exemplary amino acid sequences of WW domain containing proteins (WW domains underlined) are listed below. It should be appreciated that any of the WW domains or WW domain variants of the exemplary proteins may be used in the invention, described herein, and are not meant to be limiting.

Human WWP1 amino acid sequence (uniprot.org/uniprot/Q9H0M0). The four underlined WW domains correspond to amino acids 349-382 (WW1), 381-414 (WW2), 456-489 (WW3), and 496-529 (WW4).

| (SEQ ID NO: 6) | |
|---|---|
| MATASPRSDT SNNHSGRLQL QVTVSSAKLK RKKNWFGTAI YTEVVVDGEI | 50 |
| TKTAKSSSSS NPKWDEQLTV NVTPQTTLEF QVWSHRTLKA DALLGKATID | 100 |
| LKQALLIHNR KLERVKEQLK LSLENKNGIA QTGELTVVLD GLVIEQENIT | 150 |
| NCSSSPTIEI QENGDALHEN GEPSARTTAR LAVEGINGID NHVPTSTIVQ | 200 |
| NSCCSYVVNG DNTPSSPSQV AARPKNTPAP KPLASEPADD TVNGESSSFA | 250 |
| PTDNASVIGT PVVSEENALS PNCTSTTVED PPVQEILTSS ENNECIPSTS | 300 |
| AELESEARSI LEPDTSNSRS SSAFEAAKSR QPDGCMDPVR QQSGNANTET | 350 |
| LPSGWEQRKD PHGRTYYVDH NTRITTWERP QPLPPGWERR VDDRRRVYYV | 400 |
| DHNTRITTWQ RPTMESVRNF EQWQSQRNQL QGAMQQFNQR YLYSASMLAA | 450 |
| ENDPYGPLPP GWEKRVDSTD RVYFVNHNTK TTQWEDPRTQ GLQNEEPLPE | 500 |
| GWEIRYTREG VRYFVDHNTR TTTFKDPRNG KSSVTKGGPQ IAYERGFRWK | 550 |
| LAHFRYLCQS NALPSHVKIN VSRQTLFEDS FQQIMALKPY DLRRRLYVIF | 600 |
| RGEEGLDYGG LAREWFFLLS HEVLNPMYCL FEYAGKNNYC LQINPASTIN | 650 |
| PDHLSYFCFI GRFIAMALFH GKFIDTGFSL PFYKRMLSKK LTIKDLESID | 700 |
| TEFYNSLIWI RDNNIEECGL EMYFSVDMEI LGKVTSHDLK LGGSNILVTE | 750 |
| ENKDEYIGLM TEWRFSRGVQ EQTKAFLDGF NEVVPLQWLQ YEDEKELEVM | 800 |
| LCGMQEVDLA DWQRNTVYRH YTRNSKQIIW FWQFVKETDN EVRMRLLQFV | 850 |
| TGTCRLPLGG FAELMGSNGP QKFCIEKVGK DTWLPRSHTC FNRLDLPPYK | 900 |
| SYEQLKEKLL FAIEETEGFG QE | 922 |

WW1(349-382):
(SEQ ID NO: 36)
ETLPSGWEQRKDPHGRTYYVDHNTRTTTWERPQP.
WW2 (381-414):
(SEQ ID NO: 37)
QPLPPGWERRVDDRRRVYYVDHNTRTTTWQRPTM.
WW3 (456-489):
(SEQ ID NO: 38)
ENDPYGPLPPGWEKRVDSTDRVYFVNHNTKTTQWEDPRT.
WW4 (496-529):
(SEQ ID NO: 39)
EPLPEGWEIRYTREGVRYFVDHNTRTTTFKDPRN.

Human WWP2 amino acid sequence (uniprot.org/uniprot/O00308). The four underlined WW domains correspond to amino acids 300-333 (WW1), 330-363 (WW2), 405-437 (WW3), and 444-547 (WW4).

| (SEQ ID NO: 7) | |
|---|---|
| MASASSSRAG VALPFEKSQL TLKVVSAKPK VHNRQPRINS YVEVAVDGLP | 50 |
| SETKKTGKRI GSSELLWNEI IILNVTAQSH LDLKVWSCHT LRNELLGTAS | 100 |
| VNLSNVLKNN GGKMENMQLT LNLQTENKGS VVSGGELTIF LDGPTVDLGN | 150 |
| VPNGSALTDG SQLPSRDSSG TAVAPENRHQ PPSINCFGGR SRTHRHSGAS | 200 |
| ARTTPATGEQ SPGARSRHRQ PVKNSGHSGL ANGTVNDEPT TATDPEEPSV | 250 |
| VGVTSPPAAP LSVTPNPNTT SLPAPATPAE GEEPSTSGTQ QLPAAAQAPD | 300 |
| ALPAGWEQRE LPNGRVYYVD HNTKTTTWER PLPPGWEKRT DPRGRFYYVD | 350 |
| HNTRITTWQR PTAEYVRNYE QWQSQRNQLQ GAMQHFSQRF LYQSSSASTD | 400 |
| HDPLGPLPPG WEKRQDNGRV YYVNHNTRTT QWEDPRTQGM IQEPALPPGW | 450 |
| EMKYTSEGVR YFVDHNTRTT TFKDPRPGFE SGTKQGSPGA YDRSFRWKYH | 500 |
| QFRFLCHSNA LPSHVKISVS RQTLFEDSFQ QIMNMKPYDL RRRLYIIMRG | 550 |
| EEGLDYGGIA REWFFLLSHE VLNPMYCLFE YAGKNNYCLQ INPASSINPD | 600 |
| HLTYFRFIGR FIAMALYHGK FIDTGFTLPF YKRMLNKRPT LKDLESIDPE | 650 |
| FYNSIVWIKE NNLEECGLEL YFIQDMEILG KVITHELKEG GESIRVTEEN | 700 |
| KEEYIMLLTD WRFTRGVEEQ TKAFLDGENE VAPLEWLRYF DEKELELMLC | 750 |
| GMQEIDMSDW QKSTIYRHYT KNSKQIQWFW QVVKEMDNEK RIRLLQFVTG | 800 |
| TCRLPVGGFA ELIGSNGPQK FCIDKVGKET WLPRSHTCEN RLDLPPYKSY | 850 |
| EQLREKLLYA IEETEGFGQE | 870 |

WW1 (300-333):
(SEQ ID NO: 40)
DALPAGWEQRELPNGRVYYVDHNTKTTTWERPLP.
WW2 (330-363):
(SEQ ID NO: 41)
PLPPGWEKRTDPRGRFYYVDHNTRTTTWQRPTA.
WW3 (405-437):
(SEQ ID NO: 42)
HDPLGPLPPGWEKRQDNGRVYYVNHNTRTTQWEDPRT.
WW4 (444-477):
(SEQ ID NO: 43)
PALPPGWEMKYTSEGVRYFVDHNTRTTTFKDPRP.

Human Nedd4-1 amino acid sequence (uniprot.org/uniprot/P46934). The four underlined WW domains correspond to amino acids 610-643 (WW1), 767-800 (WW2), 840-873 (WW3), and 892-925 (WW4).

| (SEQ ID NO: 8) | |
|---|---|
| MAQSLRLHFA ARRSNTYPLS ETSGDDLDSH VHMCFKRPTR ISTSNVVQMK | 50 |
| LTPRQTALAP LIKENVQSQE RSSVPSSENV NKKSSCLQIS LQPTRYSGYL | 100 |
| QSSNVLADSD DASFTCILKD GIYSSAVVDN ELNAVNDGHL VSSPAICSGS | 150 |
| LSNFSTSDNG SYSSNGSDFG SCASITSGGS YINSVISDSS SYTFPPSDDT | 200 |
| FLGGNLPSDS TSNRSVPNRN TTPCEIFSRS TSTDPFVQDD LEHGLEIMKL | 250 |
| PVSRNTKIPL KRYSSLVIFP RSPSTTRPTS PTSLCTLLSK GSYQTSHQFI | 300 |
| ISPSEIAHNE DGTSAKGFLS TAVNGIRLSK TICTPGEVRD IRPLHRKGSL | 350 |
| QKKIVLSNNT PRQTVCEKSS EGYSCVSVHF TQRKAATLDC ETTNGDCKPE | 400 |
| MSEIKLNSDS EYIKLMHRTS ACLPSSQNVD CQININGELE RPHSQMNKNH | 450 |
| GILRRSISIG GAYPNISCLS SLKHNCSKGG PSQLLIKFAS GNEGKVDNLS | 500 |
| RDSNRDCTNE LSNSCKTRDD FLGQVDVPLY PLPTENPRLE RPYTFKDFVL | 550 |
| HPRSHKSRVK GYLRLKMTYL PKTSGSEDDN AEQAEELEPG WVVLDQPDAA | 600 |
| CHLQQQQEPS PLPPGWEERQ DILGRTYYVN HESRRTQWKR PTPQDNLTDA | 650 |
| ENGNIQLQAQ RAFTTRRQIS EETESVDNRE SSENWEIIRE DEATMYSNQA | 700 |
| FPSPPPSSNL DVPTHLAEEL NARLTIFGNS AVSQPASSSN HSSRRGSLQA | 750 |
| YTFEEQPTLP VLLPTSSGLP PGWEEKQDER GRSYYVDHNS RITTWTKPTV | 800 |
| QATVETSQLT SSQSSAGPQS QASTSDSGQQ VTQPSEIEQG FLPKGWEVRH | 850 |
| APNGRPFFID HNTKTTTWED PRLKIPAHLR GKTSLDTSND LGPLPPGWEE | 900 |
| RTHTDGRIFY INHNIKRTQW EDPRLENVAI TGPAVPYSRD YKRKYEFFRR | 950 |
| KLKKQNDIPN KFEMKLRRAT VLEDSYRRIM GVKRADELKA RLWIEFDGEK | 1000 |
| GLDYGGVARE WFFLISKEMF NPYYGLFEYS ATDNYTLQIN PNSGLCNEDH | 1050 |
| LSYFKFIGRV AGMAVYHGKL LDGFFIRPFY KMMLHKPITL HDMESVDSEY | 1100 |
| YNSLRWILEN DPTELDLRFI IDEELFGQTH QHELKNGGSE IVVINKNKKE | 1150 |
| YIYLVIQWRF VNRIQKQMAA FKEGFFELIP QDLIKIFDEN ELELLMCGLG | 1200 |
| DVDVNDWREH TKYKNGYSAN HQVIQWFWKA VIMMDSEKRI RLLQFVTGTS | 1250 |
| RVPMNGFAEL YGSNGPQSFT VEQWGTPEKL PRAHTCFNRL DLPPYESFEE | 1300 |
| LWDKLQMAIE NTQGFDGVD | 1319 |

WW1(610-643):
(SEQ ID NO: 44)
SPLPPGWEERQDILGRTYYVNHESRRTQWKRPTP.
WW2 (767-800):
(SEQ ID NO: 45)
SGLPPGWEEKQDERGRSYYVDHNSRTTTWTKPTV.
WW3 (840-873):
(SEQ ID NO: 46)
GFLPKGWEVRHAPNGRPFFIDHNTKTTTWEDPRL.
WW4 (892-925):
(SEQ ID NO: 47)
GPLPPGWEERTHTDGRIFYINHNIKRTQWEDPRL.

Human Nedd4-2 amino acid sequence (>gi|21361472|ref|NP_056092.2|E3 ubiquitin-protein ligase NEDD4-like isoform 3 [Homo sapiens]). The four underlined WW domains correspond to amino acids 198-224 (WW1), 368-396 (WW2), 480-510 (WW3), and 531-561 (WW4).

(SEQ ID NO: 9)
MATGLGEPVYGLSEDEGESRILRVKVVSGIDLAKKDIFGASDPYVKLSLY
VADENRELALVQTKTIKKTLNPKWNEEFYFRVNPSNHRLLFEVFDENRLT
RDDFLGQVDVPLSHLPTEDPTMERPYTFKDFLLRPRSHKSRVKGFLRLKM
AYMPKNGGQDEENSDQRDDMEHGWEVVDSNDSASQHQEELPPPPLPPGWE
EKVDNLGRTYYVNHNNRTTQWHRPSLMDVSSESDNNIRQINQEAAHRRFR
SRRHISEDLEPEPSEGGDVPEPWETISEEVNIAGDSLGLALPPPPASPGS
RTSPQELSEELSRRLQITPDSNGEQFSSLIQREPSSRLRSCSVTDAVAEQ
GHLPPPSVAYVHTTPGLPSGWEERKDAKGRTYYVNHNNRTTTWTRPIMQL
AEDGASGSATNSNNHLIEPQIRRPRSLSSPTVTLSAPLEGAKDSPVRRAV
KDTLSNPQSPQPSPYNSPKPQHKVTQSFLPPGWEMRIAPNGRPFFIDHNT
KTTTWEDPRLKFPVHMRSKTSLNPNDLGPLPPGWEERIHLDGRTFYIDHN
SKITQWEDPRLQNPAITGPAVPYSREFKQKYDYFRKKLKKPADIPNRFEM
KLHRNNIFEESYRRIMSVKRPDVLKARLWIEFESEKGLDYGGVAREWFFL
LSKEMFNPYYGLFEYSATDNYTLQINPNSGLCNEDHLSYFTFIGRVAGLA
VFHGKLLDGFFIRPFYKMMLGKQITLNDMESVDSEYYNSLKWILENDPTE
LDLMFCIDEENFGQTYQVDLKPNGSEIMVTNENKREYIDLVIQWRFVNRV
QKQMNAFLEGFTELLPIDLIKIFDENELELLMCGLGDVDVNDWRQHSIYK
NGYCPNHPVIQWFWKAVLLMDAEKRIRLLQFVTGTSRVPMNGFAELYGSN
GPQLFTIEQWGSPEKLPRAHTCFNRLDLPPYETFEDLREKLLMAVENAQG
FEGVD
WW1(198-224):
(SEQ ID NO: 61)
GWEEKVDNLGRTYYVNHNNRTTQWHRP.
WW2 (368-396):
(SEQ ID NO: 62)
PSGWEERKDAKGRTYYVNHNNRTTTWTRP.
WW3 (480-510):
(SEQ ID NO: 63)
PPGWEMRIAPNGRPFFIDHNTKTTTWEDPRL.
WW4 (531-561):
(SEQ ID NO: 64)
PPGWEERIHLDGRTFYIDHNSKITQWEDPRL.

Human Smurf1 amino acid sequence (uniprot.org/uniprot/Q9HCE7). The two underlined WW domains correspond to amino acids 234-267 (WW1) and 306-339 (WW2).

| (SEQ ID NO: 10) | |
|---|---|
| MSNPGTRRNG SSIKIRLIVL CAKNLAKKDF FRLPDPFAKI VVDGSGQCHS | 50 |
| TDTVKNTLDP KWNQHYDLYV GKTDSITISV WNHKKIHKKQ GAGFLGCVRL | 100 |
| LSNAISRLKD TGYQRLDLCK LNPSDTDAVR GQIVVSLQTR DRIGTGGSVV | 150 |
| DCRGLLENEG TVYEDSGPGR PLSCFMEEPA PYTDSTGAAA GGGNCRFVES | 200 |
| PSQDQRLQAQ RLRNPDVRGS LQTPQNRPHG HQSPELPEGY EQRTTVQGQV | 250 |
| YFLHTQTGVS TWHDPRIPSP SGTIPGGDAA FLYEFLLQGH TSEPRDLNSV | 300 |
| NCDELGPLPP GWEVRSTVSG RIYFVDHNNR TTQFTDPRLH HIMNHQCQLK | 350 |
| EPSQPLPLPS EGSLEDEELP AQRYERDLVQ KLKVLRHELS LQQPQAGHCR | 400 |
| IEVSREEIFE ESYRQIMKMR PKDLKKRLMV KFRGEEGLDY GGVAREWLYL | 450 |
| LCHEMLNPYY GLFQYSTDNI YMLQINPDSS INPDHLSYFH FVGRIMGLAV | 500 |
| FHGHYINGGF TVPFYKQLLG KPIQLSDLES VDPELHKSLV WILENDITPV | 550 |
| LDHTFCVEHN AFGRILQHEL KPNGRNVPVT EENKKEYVRL YVNWRFMRGI | 600 |
| EAQFLALQKG FNELIPQHLL KPFDQKELEL IIGGLDKIDL NDWKSNTRLK | 650 |
| HCVADSNIVR WFWQAVETED EERRARLLQF VTGSTRVPLQ GFKALQGSTG | 700 |
| AAGPRLFTIH LIDANTDNLP KAHTCFNRID IPPYESYEKL YEKLLTAVEE | 750 |
| TCGFAVE | 757 |

WW1 (234-267):
(SEQ ID NO: 48)
PELPEGYEQRTTVQGQVYFLHTQTGVSTWHDPRI.
WW2 (306-339):
(SEQ ID NO: 49)
GPLPPGWEVRSTVSGRIYFVDHNNRTTQFTDPRL.

Human Smurf2 amino acid sequence (uniprot.org/uniprot/Q9HAU4). The three underlined WW domains correspond to amino acids 157-190 (WW1), 251-284 (WW2), and 297-330 (WW3).

| (SEQ ID NO: 11) | |
|---|---|
| MSNPGGRRNG PVKLRLTVLC AKNLVKKDFF RLPDPFAKVV VDGSGQCHST | 50 |
| DTVKNTLDPK WNQHYDLYIG KSDSVTISVW NHKKIHKKQG AGFLGCVRLL | 100 |
| SNAINRIKDT GYQRLDLCKL GPNDNDTVRG QIVVSLQSRD RIGTGGQVVD | 150 |
| CSRLFDNDLP DGWEERRTAS GRIQYLNHIT RTTQWERPTR PASEYSSPGR | 200 |
| PLSCFVDENT PISGINGATC GQSSDPRLAE RRVRSQRHRN YMSRTHLHTP | 250 |
| PDLPEGYEQR TTQQGQVYFL HTQTGVSTWH DPRVPRDLSN INCEELGPLP | 300 |
| PGWEIRNTAT GRVYFVDHNN RTTQFTDPRL SANLHLVLNR QNQLKDQQQQ | 350 |
| QVVSLCPDDT ECLTVPRYKR DLVQKLKILR QELSQQQPQA GHCRIEVSRE | 400 |
| EIFEESYRQV MKMRPKDLWK RLMIKFRGEE GLDYGGVARE WLYLLSHEML | 450 |
| NPYYGLFQYS RDDIYTLQIN PDSAVNPEHL SYFHFVGRIM GMAVFHGHYI | 500 |
| DGGFTLPFYK QLLGKSITLD DMELVDPDLH NSLVWILEND ITGVLDHTFC | 550 |
| VEHNAYGEII QHELKPNGKS IPVNEENKKE YVRLYVNWRF LRGIEAQFLA | 600 |
| LQKGFNEVIP QHLLKIFDEK ELELIICGLG KIDVNDWKVN TRLKHCTPDS | 650 |
| NIVKWFWKAV EFFDEERRAR LLQFVTGSSR VPLQGFKALQ GAAGPRLFTI | 700 |
| HQIDACINNL PKAHTCFNRI DIPPYESYEK LYEKLLTAIE ETCGFAVE | 748 |

WWI (157-190):
(SEQ ID NO: 50)
NDLPDGWEERRTASGRIQYLNHITRTTQWERPTR.
WW2 (251-284):
(SEQ ID NO: 51)
PDLPEGYEQRTTQQGQVYFLHTQTGVSTWHDPRV.
WW3 (297-330):
(SEQ ID NO: 52)
GPLPPGWEIRNTATGRVYFVDHNNRTTQFTDPRL.

Human ITCH amino acid sequence (uniprot.org/uniprot/Q96J02). The four underlined WW domains correspond to amino acids 326-359 (WW1), 358-391 (WW2), 438-471 (WW3), and 478-511 (WW4).

| (SEQ ID NO: 12) | |
|---|---|
| MSDSGSQLGS MGSLTMKSQL QITVISAKLK ENKKNWFGPS PYVEVTVDGQ | 50 |
| SKKTEKCNNT NSPKWKQPLT VIVTPVSKLH FRVWSHQTLK SDVLLGTAAL | 100 |
| DIYETLKSNN MKLEEVVVTL QLGGDKEPTE TIGDLSICLD GLQLESEVVT | 150 |
| NGETTCSENG VSLCLPRLEC NSAISAHCNL CLPGLSDSPI SASRVAGFTG | 200 |
| ASQNDDGSRS KDETRVSING SDDPEDAGAG ENRRVSGNNS PSLSNGGFKP | 250 |
| SRPPRPSRPP PPTPRRPASV NGSPSATSES DGSSTGSLPP TNTNTNTSEG | 300 |
| ATSGLIIPLT ISGGSGPRPL NPVTQAPLPP GWEQRVDQHG RVYYVDHVEK | 350 |
| RTTWDRPEPL PPGWERRVDN MGRIYYVDHF TRITTWQRPT LESVRNYEQW | 400 |
| QLQRSQLQGA MQQFNQRFIY GNQDLFATSQ SKEFDPLGPL PPGWEKRTDS | 450 |
| NGRVYFVNHN TRITQWEDPR SQGQLNEKPL PEGWEMRFTV DGIPYFVDHN | 500 |
| RRITTYIDPR TGKSALDNGP QIAYVRDFKA KVQYFRFWCQ QLAMPQHIKI | 550 |
| TVTRKILFED SFQQIMSFSP QDLRRRLWVI FPGEEGLDYG GVAREWFFLL | 600 |
| SHEVLNPMYC LFEYAGKDNY CLQINPASYI NPDHLKYFRF IGRFIAMALF | 650 |
| HGKFIDTGFS LPFYKRILNK PVGLKDLESI DPEFYNSLIW VKENNIEECD | 700 |
| LEMYFSVDKE ILGEIKSHDL KPNGGNILVT EENKEEYIRM VAEWRLSRGV | 750 |
| EEQTQAFFEG FNEILPQQYL QYFDAKELEV LLCGMQEIDL NDWQRHAIYR | 800 |
| HYARTSKQIM WFWQFVKEID NEKRMRLLQF VIGTCRLPVG GFADLMGSNG | 850 |
| PQKFCIEKVG KENWLPRSHT CFNRLDLPPY KSYEQLKEKL LFAIEETEGF | 900 |
| GQE | 903 |

ITCH WWI (326-359):
(SEQ ID NO: 53)
APLPPGWEQRVDQHGRVYYVDHVEKRTTWDRPEP.
ITCH WW2 (358-391):
(SEQ ID NO: 54)
EPLPPGWERRVDNMGRIYYVDHFTRTTTWQRPTL.
ITCH WW3 (438-471):
(SEQ ID NO: 55)
GPLPPGWEKRTDSNGRVYFVNHNTRITQWEDPRS.
ITCH WW4 (478-511):
(SEQ ID NO: 56)
KPLPEGWEMRFTVDGIPYFVDHNRRTTTYIDPRT.

Human NEDL1 amino acid sequence (uniprot.org/uniprot/Q76N89). The two underlined WW domains correspond to amino acids 829-862 (WW1), and 1018-1051 (WW2).

| (SEQ ID NO: 13) | |
|---|---|
| MLLHLCSVKN LYQNRFLGLA AMASPSRNSQ SRRRCKEPLR YSYNPDQFHN | 50 |
| MDLRGGPHDG VTIPRSTSDT DLVTSDSRST LMVSSSYYSI GHSQDLVIHW | 100 |
| DIKEEVDAGD WIGMYLIDEV LSENFLDYKN RGVNGSHRGQ IIWKIDASSY | 150 |
| FVEPETKICF KYYHGVSGAL RATTPSVTVK NSAAPIFKSI GADETVQGQG | 200 |
| SRRLISFSLS DFQAMGLKKG MFFNPDPYLK ISIQPGKHSI FPALPHHGQE | 250 |
| RRSKIIGNTV NPIWQAEQFS FVSLPTDVLE IEVKDKFAKS RPIIKRFLGK | 300 |
| ISMPVQRLLE RHAIGDRVVS YILGRRLPTD HVSGQLQFRF EITSSIHPDD | 350 |
| EEISLSTEPE SAQIQDSPMN NLMESGSGEP RSEAPESSES WKPEQLGEGS | 400 |
| VPDGPGNQSI ELSRPAEEAA VITEAGDQGM VSVGPEGAGE LLAQVQKDIQ | 450 |
| PAPSAEELAE QLDLGEEASA LLLEDGEAPA STKEEPLEEE ATTQSRAGRE | 500 |
| EEEKEQEEEG DVSTLEQGEG RLQLRASVKR KSRPCSLPVS ELETVIASAC | 550 |
| GDPETPRTHY IRIHTLLHSM PSAQGGSAAE EEDGAEEEST LKDSSEKDGL | 600 |
| SEVDTVAADP SALEEDREEP EGATPGTAHP GHSGGHFPSL ANGAAQDGDT | 650 |
| HPSTGSESDS SPRQGGDHSC EGCDASCCSP SCYSSSCYST SCYSSSCYSA | 700 |
| SCYSPSCYNG NRFASHTRES SVDSAKISES TVFSSODDEE EENSAFESVP | 750 |
| DSMQSPELDP ESTNGAGPWQ DELAAPSGHV ERSPEGLESP VAGPSNRREG | 800 |
| ECPILHNSQP VSQLPSLRPE HHHYPTIDEP LPPNWEARID SHGRVFYVDH | 850 |
| VNRITTWQRP TAAATPDGMR RSGSIQQMEQ LNRRYQNIQR TIATERSEED | 900 |
| SGSQSCEQAP AGGGGGGGSD SEAESSQSSL DLRREGSLSP VNSQKITLLL | 950 |
| QSPAVKFIIN PEFFTVLHAN YSAYRVFTSS TCLKHMILKV RRDARNFERY | 1000 |
| QHNRDLVNFI NMFADTRLEL PRGWEIKTDQ QGKSFFVDHN SRATTFIDPR | 1050 |
| IPLONGRLPN HLTHROHLOR LRSYSAGEAS EVSRNRGASL LARPGHSLVA | 1100 |
| AIRSQHOHES LPLAYNDKIV AFLROPNIFE MLQEROPSLA RNHTLREKIH | 1150 |
| YIRTEGNHGL EKLSCDADLV ILLSLFEEEI MSYVPLQAAF HPGYSFSPRC | 1200 |
| SPCSSPONSP GLORASARAP SPYRRDFEAK LRNFYRKLEA KGFGQGPGKI | 1250 |
| KLIIRRDHLL EGTFNQVMAY SRKELORNKL YVTFVGEEGL DYSGPSREFF | 1300 |
| FLLSQELFNP YYGLFEYSAN DTYTVQISPM SAFVENHLEW FRFSGRILGL | 1350 |
| ALIHQYLLDA FFTRPFYKAL LRLPCDLSDL EYLDEEFHQS LOWMKDNNIT | 1400 |
| DILDLIFTVN EEVFGQVTER ELKSGGANTQ VTEKNKKEYI ERMVKWRVER | 1450 |
| GVVQQTEALV RGFYEVVDSR LVSVFDAREL ELVIAGTAEI DLNDWRNNTE | 1500 |
| YRGGYHDGHL VIRWFWAAVE RENNEQRLRL LQFVTGTSSV PYEGFAALRG | 1550 |
| SNGLRRFCIE KWGKITSLPR AHTCFNRLDL PPYPSYSMLY EKLLTAVEET | 1600 |
| STFGLE | 1606 |

WW1 (829-862)
(SEQ ID NO: 57)
PLPPNWEARIDSHGRVFYVDHVNRTTTWQRPTA.
WW2 (1018-1051):
(SEQ ID NO: 58)
LELPRGWEIKTDQQGKSFFVDHNSRATTFIDPRI.

Human NEDL2 amino acid sequence (uniprot.org/uniprot/Q9P2P5). The two underlined WW domains correspond to amino acids 807-840 (WW1) and 985-1018 (WW2).

| (SEQ ID NO: 14) | |
|---|---|
| MASSAREHLL FVRRRNPQMR YILSPENLQS LAAQSSMPEN MTLQRANSDT | 50 |
| DLVISESRSS LTASMYEYTL GQAQNLIIFW DIKEEVDPSD WIGLYHIDEN | 100 |
| SPANFWDSKN RGVTGTQKGQ IVWRIEPGPY FMEPEIKICF KYYHGISGAL | 150 |
| RATTPCITVK NPAVMMGAEG MEGGASGNLH SRKLVSFTLS DLRAVGLKKG | 200 |
| MFFNPDPYLK MSIQPGKKSS FPTCAHHGQE RRSTIISNTT NPIWHREKYS | 250 |
| FFALLTDVLE IEIKDKFAKS RPIIKRFLGK LTIPVQRLLE RQAIGDQMLS | 300 |
| YNLGRRLPAD HVSGYLQFKV EVISSVHEDA SPEAVGTILG VNSVNGDLGS | 350 |
| PSDDEDMPGS HHDSQVCSNG PVSEDSAADG TPKHSFRISS TLEIDTEELT | 400 |
| SISSRISPPR GRQDSLNDYL DAIEHNGHSR PGTATCSERS MGASPKLRSS | 450 |
| FPTDTRLNAM LHIDSDEEDH EFQQDLGYPS SLEEEGGLIM FSRASRADDG | 500 |
| SLTSQTKLED NPVENEEAST HEAASFEDKP ENLPELAESS LPAGPAPEEG | 550 |
| EGGPEPQPSA DQGSAELCGS QEVDQPTSGA DTGTSDASGG SRRAVSETES | 600 |
| LDQGSEPSQV SSETEPSDPA RTESVSEAST RPEGESDLEC ADSSCNESVT | 650 |
| TQLSSVDTRC SSLESARFPE TPAFSSQEEE DGACAAEPTS SGPAEGSQES | 700 |
| VCTAGSLPVV QVPSGEDEGP GAESATVPDQ EELGEVWQRR GSLEGAAAAA | 750 |
| ESPPQEEGSA GEAQGTCEGA TAQEEGATGG SQANGHQPLR SLPSVRQDVS | 800 |
| RYQRVDEALP PNWEARIDSH GRIFYVDHVN RITTWQRPTA PPAPQVLQRS | 850 |
| NSIQQMEQLN RRYQSIRRTM TNERPEENTN AIDGAGEEAD FHQASADERR | 900 |
| ENILPHSTSR SRITLLLQSP PVKFLISPEF FTVLHSNPSA YRMFINNTCL | 950 |
| KHMITKVRRD THHFERYQHN RDLVGFLNMF ANKQLELPRG WEMKHDHQGK | 1000 |
| AFFVDHNSRT TTFIDPRLPL QSSRPTSALV HRQHLTRQRS HSAGEVGEDS | 1050 |
| RHAGPPVLPR PSSTENTVSR PQYQDMVPVA YNDKIVAFLR QPNIFEILQE | 1100 |
| RQPDLTRNHS LREKIQFIRT EGTPGLVRLS SDADLVMLLS LFEEEIMSYV | 1150 |
| PPHALLHPSY CQSPRGSPVS SPQNSPGTQR ANARAPAPYK RDFEAKLRNF | 1200 |
| YRKLETKGYG QGPGKLKLII RRDHLLEDAF NQIMGYSRKD LQRNKLYVTF | 1250 |
| VGEEGLDYSG PSREFFFLVS RELFNPYYGL FEYSANDTYT VQISPMSAFV | 1300 |
| DNHHEWERFS GRILGLALIH QYLLDAFFTR PFYKALLRIL CDLSDLEYLD | 1350 |
| EEFHQSLQWM KDNDIHDILD LIFTVNEEVF GQITERELKP GGANIPVTEK | 1400 |
| NKKEYIERMV KWRIERGVVQ QTESLVRGFY EVVDARLVSV FDARELELVI | 1450 |
| AGTAEIDLSD WRNNTEYRGG YHDNHIVIRW FWAAVERFNN EQRLRLLQFV | 1500 |
| TGTSSIPYEG FASLRGSNGP RRFCVEKWGK ITALPRAHTC FNRLDLPPYP | 1550 |
| SFSMLYEKLL TAVEETSTFG LE | 1572 |

WWI (807-840):
(SEQ ID NO: 59)
EALPPNWEARIDSHGRIFYVDHVNRTTTWQRPTA.
WW2 (985-1018):
(SEQ ID NO: 60)
LELPRGWEMKHDHQGKAFFVDHNSRTTTFIDPRL.

In some embodiments, the WW domain comprises a WW domain or WW domain variant from the amino acid sequence (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14). In other embodiments, the WW domain consists of a WW domain or WW domain variant from the amino acid sequence (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14). In another embodiment, the WW domain consists essentially of a WW domain or WW domain variant from the amino acid sequence (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14). Consists essentially of means that a domain, peptide, or polypeptide consists essentially of an amino acid sequence when such an amino acid sequence is present with only a few additional amino acid residues, for example, from about 1 to about 10 or so additional residues, typically from 1 to about 5 additional residues in the domain, peptide, or polypeptide.

Alternatively, the WW domain may be a WW domain that has been modified to include two basic amino acids at the C-terminus of the domain. Techniques are known in the art and are described in the art, for example, in Sambrook et al. ((2001) Molecular Cloning: a Laboratory Manual, 3rd ed., Cold Spring Harbour Laboratory Press). Thus, a skilled person could readily modify an existing WW domain that does not normally have two C-terminal basic residues so as to include two basic residues at the C-terminus.

Basic amino acids are amino acids that possess a side-chain functional group that has a pKa of greater than 7 and includes lysine, arginine, and histidine, as well as basic amino acids that are not included in the twenty α-amino acids commonly included in proteins. The two basic amino acids at the C-terminus of the WW domain may be the same basic amino acid or may be different basic amino acids. In one embodiment, the two basic amino acids are two arginines.

The term WW domain also includes variants of a WW domain provided that any such variant possesses two basic amino acids at its C-terminus and maintains the ability of the WW domain to associate with the PPXY (SEQ ID NO: 2) motif. A variant of such a WW domain refers to a WW domain which retains the ability of the variant to associate with the PPXY (SEQ ID NO: 2) motif (i.e., the PPXY (SEQ ID NO: 2) motif of ARRDC1) and that has been mutated at one or more amino acids, including point, insertion, and/or deletion mutations, but still retains the ability to associate with the PPXY (SEQ ID NO: 2) motif. A variant or derivative therefore includes deletions, including truncations and fragments; insertions and additions, for example conservative substitutions, site-directed mutants and allelic variants; and modifications, including one or more non-amino acyl groups (e.g., sugar, lipid, etc.) covalently linked to the peptide and post-translational modifications. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The WW domain may be part of a longer protein. Thus, the protein, in various different embodiments, comprises the WW domain, consists of the WW domain or consists essentially of the WW domain, as defined herein. The polypeptide may be a protein that includes a WW domain as a functional domain within the protein sequence. In some embodiments, the polypeptide comprises the sequence set forth in (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14), consists of (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14), or consists essentially of (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14).

### DETAILED DESCRIPTION OF CERTAIN EMBODIMENTS OF THE INVENTION

The instant disclosure relates, at least in part, to the discovery that a GFP-encoding cargo RNA fused to a TAR element can be loaded into ARMMs by co-expressing the TAR:cargo RNA fusion with an ARRDC1:Tat fusion protein in a cell. The disclosure also demonstrates that ARMMs containing a GFP-encoding cargo RNA were able to deliver their GFP-encoding cargo RNA into targeted cells. Furthermore, fusing of the TAR element with the GFP-encoding cargo RNA did not inhibit GFP expression from the cargo RNA. As described in more detail herein, cargo RNAs (e.g., RNAs that encode proteins (e.g., therapeutic proteins) or siRNAs that inhibit the expression of one or more proteins) may be associated (covalently or non-covalently) with one or more binding RNAs (e.g., a TAR element) in order to facilitate loading of the cargo RNA into an ARMM, for example, by binding to an ARMM protein (e.g., ARRDC1 or fragment thereof). Loading a cargo RNA into an ARMM may be performed by expressing an ARRDC1 protein, or fragment thereof, fused to a RNA binding protein (e.g., Tat), or fragment thereof, so that a cargo RNA associated with a binding RNA (e.g., TAR element) binds to the fusion protein of ARRDC1:RNA binding protein and is loaded into an ARMM. Alternatively, a fusion protein, such as an RNA binding protein:WW domain fusion protein (e.g., Tat:WW), may be used to recruit a cargo RNA associated with a binding RNA (e.g., a TAR element) to ARRDC1 in order to load the cargo RNA into an ARMM. For example, a cargo RNA associated with a TAR element may bind to the Tat portion of a Tat:WW fusion protein. The WW domain of the Tat:WW fusion protein may bind to ARRDC1 (e.g., via the PPXY (SEQ ID NO: 2) motif of ARRDC1), thereby recruiting the cargo RNA into an ARMM by associating it with the ARMM protein ARRDC1.

ARMMs containing cargo RNAs, such as RNAs that express therapeutic proteins or siRNAs that inhibit the expression of one or more proteins, may be used to deliver the cargo RNA to a cell. The ARMMs may be delivered to cells in vitro or in vivo. For example, ARMMs may be incubated with cells in culture (e.g., by adding them to the cell culture medium) in order to deliver the contents of the ARMMs into the cultured cells. As another example, ARMMs may be delivered to the cells of a subject, e.g., by administering the ARMMs to the subject. ARMMs may also be modified to target one or more cell types. For example, ARMMs may be associated with one or more binding agents that selectively bind an antigen on the surface of the target cell. Methods for producing membrane-bound binding agents, for example, membrane-bound immunoglobulins, membrane-bound antibodies or antibody fragments that specifically bind a surface antigen expressed on the surface of cells (e.g., cancer cells), are known to those of skill in the art. Cell surface antigens specifically expressed on various types of cells that can be targeted by ARMMs comprising membrane-bound binding agents in order to deliver the contents of the ARMMs into one or more targeted cells.

Microvesicles with ARRDC1 and Binding RNAs

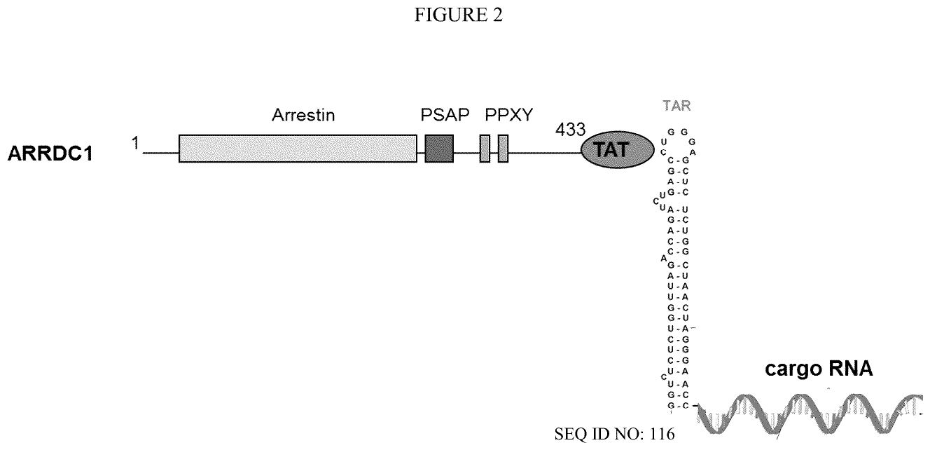

Some aspects of this invention provide arrestin domain-containing protein 1 (ARRDC1)-mediated microvesicles (ARMMs) containing an ARRDC1 protein, or variant thereof, associated with a binding RNA. The binding RNA may associate with the ARRDC1 protein in different ways. For example, the ARRDC1 may be fused to an RNA binding protein, or variant thereof, that associates with the binding RNA, thereby associating the binding RNA with the ARRDC1 via the RNA binding protein. See, for example, the schematic of FIG. 2 showing AARDC1 fused to a Tat protein, which associates with a TAR binding RNA. As another example, an ARMM may comprise an RNA binding protein fused to one or more WW domains, which associates with ARRDC1 via at least one WW domain and also associates with a binding RNA via the RNA binding protein, thereby associating the binding RNA with ARRDC1.

Some aspects of this invention provide arrestin domain-containing protein 1 (ARRDC1)-mediated microvesicles (ARMMs) containing an ARRDC1 protein, or variant thereof, that is associated with an RNA binding protein, or variant thereof, and a binding RNA that is associated with the RNA binding protein. Such ARMMs typically include a lipid bilayer and an ARRDC1 protein or variant thereof. In some embodiments, the ARRDC1 protein is non-covalently associated with the RNA binding protein. In some embodiments, ARRDC1 protein is covalently associated with the RNA binding protein. In some embodiments, the RNA binding protein is fused to the N-terminus of the ARRDC1 protein. In some embodiments, the RNA binding protein is fused to the C-terminus of the ARRDC1 protein. In some embodiments, the RNA binding protein is non-covalently associated with the binding RNA.

Some aspects of this invention provide arrestin domain-containing protein 1 (ARRDC1)-mediated microvesicles (ARMMs) containing an ARRDC1 protein, or variant thereof, and an RNA binding protein fused to at least one WW domain, or variant thereof, and a binding RNA that is associated with the RNA binding protein. Such ARMMs typically include a lipid bilayer and an ARRDC1 protein, or variant thereof. In some embodiments, the RNA binding protein fused to a WW domain associates with the PPXY (SEQ ID NO: 2) (where x=any amino acid) domain of ARRDC1, via the WW domain, which may facilitate loading of the binding RNA into an ARMM. In some embodiments, at least one WW domain is fused to the N-terminus of an RNA binding protein. In some embodiments, 1, 2, 3, 4, 5, 6, 7, 8, 9, or 10 WW domains are fused to the N-terminus of an RNA binding protein. In some embodiments, at least one WW domain is fused to the C-terminus of an RNA binding protein. In some embodiments, 1, 2, 3, 4, 5, 6, 7, 8, 9, or 10 WW domains are fused to the C-terminus of an RNA binding protein.

In some embodiments, the binding RNA is associated with a cargo RNA, which may facilitate loading of the cargo RNA into an ARMM. In some embodiments, the binding RNA is covalently associated with the cargo RNA. In some embodiments, the binding RNA and the cargo RNA are part of the same RNA molecule (e.g., an RNA from a single transcript). In some embodiments, the binding RNA and the cargo RNA are covalently associated via a linker. In some embodiments, the linker comprises a nucleotide or nucleic acid (e.g., DNA or RNA). In some embodiments, the linker comprises RNA. In some embodiments, the linker comprises DNA. In some embodiments, the linker comprises at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, at least 200, at least 250, at least 300, at least 400, or at least 500 nucleotides (e.g., DNA or RNA).

In other embodiments, the binding RNA is non-covalently associated with the cargo RNA. For example, the binding RNA may associate with the cargo RNA via complementary base pairing. In some embodiments, the binding RNA is bound to the cargo RNA via at least 2, at least 3, at least 4, at least 5, at least 6, at least 7, at least 8, at least 9, at least 10, at least 15, at least 20, at least 25, at least 30, at least 40, at least 50 complementary base pairs, which may be contiguous or non-contiguous. In some embodiments, the binding RNA is bound to the cargo RNA via at least 2, at least 3, at least 4, at least 5, at least 6, at least 7, at least 8, at least 9, at least 10, at least 15, at least 20, at least 25, at least 30, at least 40, or at least 50 contiguous complementary base pairs.

It should be appreciated that any number of cargo RNAs can be associated with a binding RNA, for example, to facilitate loading of the cargo RNA into an ARMM. A cargo RNA may, for example, encode a reprogramming factor (e.g., Oct4, Sox2, c-Myc, or KLF4), which may be loaded into an ARMM by associating it with an ARRDC1 fused to an RNA binding protein via a binding RNA. In some embodiments, the cargo RNA is an mRNA that encodes a therapeutic protein (e.g., a transcription factor, a tumor suppressor, a developmental regulator, a growth factor, a metastasis suppressor, a pro-apoptotic protein, a zinc finger nuclease, or a recombinase). In other embodiments, the cargo RNA is an siRNA that inhibits expression of a protein (e.g., a transcription factor, a tumor suppressor, a developmental regulator, a growth factor, a metastasis suppressor, a metastasis promoter, an oncogene, a pro-apoptotic protein, a zinc finger nuclease, or a recombinase). In other embodiments, an ARMM further includes a TSG101 protein, or variant thereof, to facilitate the release of ARMMs. Without wishing to be bount by any particular theory, the TSG101 protein interacts with ARRDC1, which results in relocation of TSG101 from endosomes to the plasma membrane and mediates the release of microvesicles that contain TSG101, ARRDC1, and other cellular components, including, for example, cargoRNAs (e.g., TAR:cargoRNA) and RNA binding proteins (e.g., ARRDC1:Tat).

### ARRDC1

ARRDC1 is a protein that comprises a PSAP (SEQ ID NO: 1) motif and a PPXY (SEQ ID NO: 2) motif, also referred to herein as a PSAP (SEQ ID NO: 1) and PPXY (SEQ ID NO: 2) motif, respectively, in its C-terminus, and interacts with TSG101 as shown herein. It should be appreciated that the PSAP (SEQ ID NO: 1) motif and the PPXY (SEQ ID NO: 2) motif are not required to be at the absolute C-terminal end of the ARRDC1. Rather, they may be at a C-terminal portion of the ARRDC1 protein (e.g., the C-terminal half of the ARRDC1). The disclosure also contemplates variants of ARRDC1, such as fragments of ARRDC1 and/or ARRDC1 proteins that have a degree of identity (e.g., 60%, 70%, 80%, 85%, 90%, 95%, 98%, or 99% identity) to an ARRDC1 protein and are capable if interacting with TSG101. Accordingly, an ARRDC1 protein may be a protein that comprises a PSAP (SEQ ID NO: 1) motif and a PPXY (SEQ ID NO: 2) motif, and interacts with TSG101. In some embodiments, the ARRDC1 protein is at least 60%, 65%, 70%, 75%, 80%, 85%, 90%, 95%, 98%, or 99% identical to the amino acid sequence of any one of SEQ ID NOs: 15-17, comprises a PSAP (SEQ ID NO: 1) motif and a PPXY (SEQ ID NO: 2) motif, and interacts with TSG101. In some embodiments, the ARRDC1 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, or at least 430 identical contiguous amino acids of any one of SEQ ID NOs: 15-17, comprises a PSAP (SEQ ID NO: 1) motif and a PPXY (SEQ ID NO: 2) motif, and interacts with TSG101. In some embodiments, the ARRDC1 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 15-17 comprises a PSAP (SEQ ID NO: 1) motif and a PPXY (SEQ ID NO: 2) motif, and interacts with TSG101. In some embodiments, the ARRDC1 protein comprises any one of the amino acid sequences set forth in SEQ ID NOs: 15-17. Exemplary, non-limiting ARRDC1 protein sequences are provided herein, and additional, suitable ARRDC1 protein variants according to aspects of this invention are known in the art. It will be appreciated by those of skill in the art that this invention is not limited in this respect. Exemplary ARRDC1 sequences include the following (PSAP (SEQ ID NO: 1) and PPXY (SEQ ID NO: 2) motifs are marked):

>gi|22748653|ref|NP_689498.1| arrestin domain-containing protein 1 [Homo sapiens]
(SEQ ID NO: 15)
MGRVQLFEISLSHGRVVYSPGEPLAGTVRVRLGAPLPFRAIRVTCIGSCGVSNKANDT
AWVVEEGYFNSSLSLADKGSLPAGEHSFPFQFLLPATAPTSFEGPFGKIVHQVRAAIH
TPRFSKDHKCSLVFYILSPLNLNSIPDIEQPNVASATKKFSYKLVKTGSVVLTASTDLR
GYVVGQALQLHADVENQSGKDTSPVVASLLQKVSYKAKRWIHDVRTIAEVEGAGV
KAWRRAQWHEQILVPALPQSALPGCSLIHIDYYLQVSLKAPEATVTLPVFIGNIAVNH

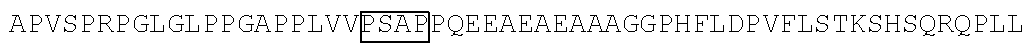

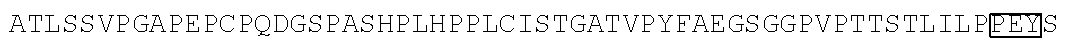

SWGYPYEAPPSYEQSCGGVEPSLTPES
>gi|244798004|ref|NP_001155957.1| arrestin domain-containing protein 1 isoform a
[Musmusculus]
(SEQ ID NO: 16)
MGRVQLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQKVSYKAKRWIYDVRTIAEVEGTGV
KAWRRAQWQEQILVPALPQSALPGCSLIHIDYYLQVSMKAPEATVTLPLFVGNIAVN

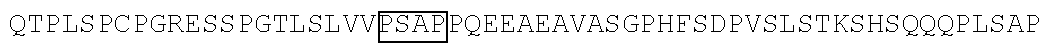

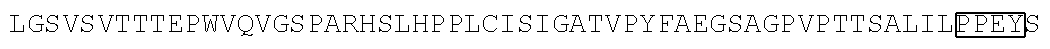

SWGYPYEAPPSYEQSCGAAGTDLGLIPGS
>gi|244798112|ref|NP_848495.2| arrestin domain-containing protein 1 isoform b
[Musmusculus]
(SEQ ID NO: 17)
MGR VQLFEIRLSQGRVVYGPGEPLAGTVHLRLGAPLPFRAIRVTCMGSCGVSTKAND
GAWVVEESYFNSSLSLADKGSLPAGEHNFPFQFLLPATAPTSFEGPFGKIVHQVRASI
DTPRFSKDHKCSLVFYILSPLNLNSIPDIEQPNVASTTKKFSYKLVKTGNVVLTASTDL
RGYVVGQVLRLQADIENQSGKDTSPVVASLLQVSYKAKRWIYDVRTIAEVEGTGVK
AWRRAQWQEQILVPALPQSALPGCSLIHIDYYLQVSMKAPEATVTLPLFVGNIAVNQ

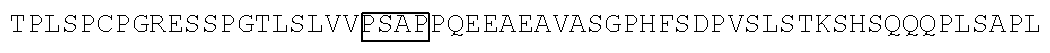

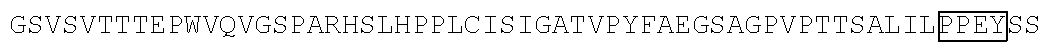

WGYPYEAPPSYEQSCGAAGTDLGLIPGS

### TSG101

In certain embodiments, the inventive microvesicles further comprise TSG101. Tumor susceptibility gene 101, also referred to herein as TSG101, is a protein encoded by this gene and belonging to a group of apparently inactive homologs of ubiquitin-conjugating enzymes. The protein contains a coiled-coil domain that interacts with stathmin, a cytosolic phosphoprotein implicated in tumorigenesis. TSG101 is a protein that comprises a UEV domain, and interacts with ARRDC1. The disclosure also contemplates variants of TSG101, such as fragments of TSG101 and/or TSG101 proteins that have a degree of identity (e.g., 60%, 70%, 80%, 85%, 90%, 95%, 98%, or 99% identity) to a TSG101 protein and are capable if interacting with ARRDC1. Accordingly, an TSG101 protein may be a protein that comprises a UEV domain, and interacts with ARRDC. In some embodiments, the TSG101 protein is at least 60%, 65%, 70%, 75%, 80%, 85%, 90%, 95%, 98%, or 99% identical to the amino acid sequence of any one of SEQ ID NOs: 20-22, comprises a UEV domain, and interacts with ARRDC1. In some embodiments, the TSG101 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390, identical contiguous amino acids of any one of SEQ ID NOs: 20-22, comprises a UEV domain, and interacts with ARRDC1. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 20-22 and comprises a UEV domain. In some embodiments, the ARRDC1 protein comprises any one of the amino acid sequences set forth in SEQ ID NOs: 20-22. Exemplary, non-limiting TSG101 protein sequences are provided herein, and additional, suitable TSG101 protein sequences, isoforms, and variants according to aspects of this invention are known in the art. It will be appreciated by those of skill in the art that this invention is not limited in this respect. Exemplary TSG101 sequences include the following:

>gi|5454140|ref|NP_006283.1| tumor susceptibility
gene 101 protein [Homo sapiens]
(SEQ ID NO: 20)
MAVSESQLKKMVSKYKYRDLTVRETVNVITLYKDLKPVLDSYVFNDGSSR
ELMNLTGTIPVPYRGNTYNIPICLWLLDTYPYNPPICFVKPTSSMTIKTG
KHVDANGKIYLPYLHEWKHPQSDLLGLIQVMIVVFGDEPPVFSRPISASY
PPYQATGPPNTSYMPGMPGGISPYPSGYPPNPSGYPGCPYPPGGPYPATT
SSQYPSQPPVTTVGPSRDGTISEDTIRASLISAVSDKLRWRMKEEMDRAQ
AELNALKRTEEDLKKGHQKLEEMVTRLDQEVAEVDKNIELLKKKDEELSS
ALEKMENQSENNDIDEVIIPTAPLYKQILNLYAEENAIEDTIFYLGEALR
RGVIDLDVFLKHVRLLSRKQFQLRALMQKARKTAGLSDLY
>gi|11230780|ref|NP_068684.1| tumor susceptibility
gene 101 protein [Mus musculus]
(SEQ ID NO: 21)
MAVSESQLKKMMSKYKYRDLTVRQTVNVIAMYKDLKPVLDSYVENDGSSR
ELVNLTGTIPVRYRGNIYNIPICLWLLDTYPYNPPICFVKPTSSMTIKTG
KHVDANGKIYLPYLHDWKHPRSELLELIQIMIVIFGEEPPVFSRPTVSAS
YPPYTATGPPNTSYMPGMPSGISAYPSGYPPNPSGYPGCPYPPAGPYPAT
TSSQYPSQPPVTTVGPSRDGTISEDTIRASLISAVSDKLRWRMKEEMDGA
QAELNALKRTEEDLKKGHQKLEEMVTRLDQEVAEVDKNIELLKKKDEELS
SALEKMENQSENNDIDEVIIPTAPLYKQILNLYAEENAIEDTIFYLGEAL
RRGVIDLDVFLKHVRLLSRKQFQLRALMQKARKTAGLSDLY
>gi|48374087|ref|NP_853659.2| tumor susceptibility
gene 101 protein [Rattus norvegicus]
(SEQ ID NO: 22)
MAVSESQLKKMMSKYKYRDLTVRQTVNVIAMYKDLKPVLDSYVFNDGSSR
ELVNLTGTIPVRYRGNIYNIPICLWLLDTYPYNPPICFVKPTSSMTIKTG
KHVDANGKIYLPYLHDWKHPRSELLELIQIMIVIFGEEPPVFSRPTVSAS
YPPYTAAGPPNTSYLPSMPSGISAYPSGYPPNPSGYPGCPYPPAGPYPAT
TSSQYPSQPPVTTAGPSRDGTISEDTIRASLISAVSDKLRWRMKEEMDGA
QAELNALKRTEEDLKKGHQKLEEMVTRLDQEVAEVDKNIELLKKKDEELS
SALEKMENQSENNDIDEVIIPTAPLYKQILNLYAEENAIEDTIFYLGEAL
RRGVIDLDVFLKHVRLLSRKQFQLRALMQKARKTAGLSDLY

The UEV domain in these sequences includes amino acids 1-145 (underlined in the sequences above). The structure of UEV domains is known to those of skill in the art (see, e.g., Owen Pornillos et al., Structure and functional interactions of the Tsg101 UEV domain, EMBO J. 2002 May 15; 21(10): 2397-2406, the entire contents of which are incorporated herein by reference).

### Fusion Proteins

### RNA Binding Proteins Fused to ARRDC1

In some aspects, microvesicles, e.g., ARMMs, are provided that comprise an ARRDC1 protein, or variant thereof, fused to an RNA binding protein, or variant thereof. In some aspects, fusion proteins are provided that comprise an ARRDC1 protein, or variant thereof, fused to a Tat protein, or variant thereof. In some aspects, expression constructs are provided that encode an ARRDC1 protein, or variant thereof, fused to an RNA binding protein (e.g., Tat), or variant thereof. In some embodiments, the ARRDC1 protein variant is a C-terminal ARRDC1 protein variant. In some embodiments, the ARRDC1 protein variant has a PSAP (SEQ ID NO: 1) motif and at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous amino acids of the ARRCD1 sequence.

Some aspects of this invention provide ARRDC1 fusion proteins that comprise an ARRDC1 protein, or a variant thereof, and an RNA binding protein, or RNA binding protein variant, associated with the ARRDC1 protein or variant thereof. In some embodiments the RNA binding protein is non-covalently linked to the ARRDC1 protein, or variant thereof. In some embodiments the RNA binding protein is covalently linked to the ARRDC1 protein, or variant thereof. The RNA binding protein, for example, may be covalently linked to the N-terminus, the C-terminus, or within the amino acid sequence of the ARRDC1 protein. In some embodiments, the ARRDC1 variant comprises a PSAP (SEQ ID NO: 1) motif (comprising the amino acid sequence PSAP (SEQ ID NO: 1)). In some embodiments, the ARRDC1 protein variant comprises the PSAP (SEQ ID NO: 1) motif and at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous amino acids of the ARRCD1 sequence.

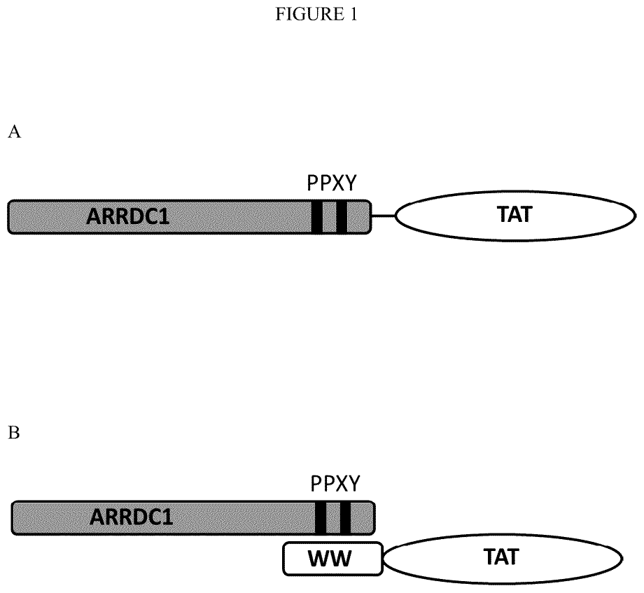

In certain embodiments, the RNA binding protein or RNA binding protein variant is fused to the C-terminus of the ARRDC1 protein or protein variant thereof. The RNA binding protein or RNA binding variant may also be fused to the N terminus of the ARRDC1 protein or variant thereof. In some embodiments, the RNA binding protein or RNA binding protein variant may be within the ARRDC1 protein or variant thereof. A schematic representation of a Tat RNA binding protein fused to the C-terminus of ARRDC1 can be seen in FIG. 1A.

In certain embodiments, the RNA binding protein is associated with an ARRDC1 protein, or variant thereof, via a covalent bond. In some embodiments, the RNA binding protein is associated with the ARRDC1 protein, or the ARRDC1 protein variant, via a linker. In some embodiments, the linker is a cleavable linker, for example, the linker may contain a protease recognition site or a disulfide bond. The protease recognition site of the linker may be recognized by a protease expressed in a target cell, resulting in the RNA binding protein fused to the ARRDC1 protein or variant thereof being released into the cytoplasm of the target cell upon uptake of the ARMM. A person skilled in the art would appreciate that any number of linkers may be used to fuse the RNA binding protein or RNA binding protein variant to the ARRDC1 protein, or variant thereof.

The linker may be cleavable or uncleavable. In some embodiments, the linker comprises an amide, ester, ether, carbon-carbon, or disulfide bond, although any covalent bond in the chemical art may be used. In some embodiments, the linker comprises a labile bond, cleavage of which results in separation of the RNA binding protein from the ARRDC1 protein, or variant thereof. In some embodiments, the linker is cleaved under conditions found in the target cell (e.g., a specific pH, a reductive environment, or the presence of a cellular enzyme). In some embodiments, the linker is cleaved by a cellular enzyme. In some embodiments, the cellular enzyme is a cellular protease or a cellular esterase. In some embodiments, the cellular enzyme is a cytoplasmic protease, an endosomal protease, or an endosomal esterase. In some embodiments, the cellular enzyme is specifically expressed in a target cell or cell type, resulting in preferential or specific release of the RNA binding protein in the target cell or cell type. The target sequence of the protease may be engineered into the linker between the RNA binding protein and the ARRDC1 protein, or variant thereof. The target cell may be any cell type found in a subject, including normal and pathologic or diseased cells, and the linker is cleaved by an enzyme or based on a characteristic specific to the target cell, or chemical environment (e.g., a cellular compartment). In some embodiments, the linker comprises an amino acid sequence chosen from the group including, but not limited to, AGVF (SEQ ID NO: 3), GFLG (SEQ ID NO: 4), FK, AL, ALAL (SEQ ID NO: 5), or ALALA (SEQ ID NO: 34). Additional linkers that may be used in accordance with the disclosure include, without limitation, those described in Chen et al., “Fusion Protein Linkers: Property, Design and Functionality” Adv Drug Deliv Rev. 2013 Oct. 15; 65(10): 1357-1369; and Choi et al., “Protease-Activated Drug Development” Theranostics, 2012; 2(2): 156-178; the entire contents of each of which are incorporated herein by reference in their entirety. Other suitable linkers will be apparent to those of skill in the art and are within the scope of this disclosure.

In some embodiments, the linker comprises a disulfide bond, which may be cleaved by reduction of the disulfide bond, for example, in vivo. In some embodiments, a disulfide bond refers to a functional group having the general structure R—S—S—R′, wherein R and R′ are alkyl groups. In some embodiments, the linker comprises one or more thiol groups. In some embodiments, the linker comprises one or more cysteine amino acid residues. In some embodiments, the disulfide bond is formed by an oxidation reaction between two cysteine residues to generate a cysteine with a disulfide bond (e.g., —S—S—). In some embodiments, the linker consists of a disulfide bond. Cleavable disulfide linkers are known in the art and have been described previously, for example, in Chen et al., “Design of an in vivo cleavable disulfide linker in recombinant fusion proteins” Biotechniques. 2010 July; 49(1): 513-518; the entire contents of which are incorporated herein by reference. However, it should be appreciated that additional cleavable linkers comprising disulfide bonds would be apparent to the skilled artisan and are within the scope of this disclosure. In some embodiments, the disulfide bond is cleaved within a cell (e.g., a target cell). As one example, any of the fusion proteins provided herein comprising a disulfide bond may be produced in a cell where the disulfide bond is not cleaved, for example, in a cell that expresses a sulfhydryl oxidase enzyme (e.g., Erv1p), which may prevent reduction of the disulfide bond. Such enzymes have been described in the art, for example, in Hatahet et al., “Disruption of reducing pathways is not essential for efficient disulfide bond formation in the cytoplasm of E. coli” Microb Cell Fact. 2010, 9: 67; the entire contents of which are incorporated herein by reference. It should be appreciated that certain cellular compartments are reducing environments (e.g., the cytosol of a cell), where the disulfide bond may be cleaved.

In some embodiments, the linker is a photo-cleavable linker. In some embodiments, the linker is a UV-cleavable moiety, which may be cleaved upon exposure to ultraviolet (UV) irradiation. Suitable photo-cleavable linkers, for example, linkers comprising a UV cleavable moiety are known to those of skill in the art. For example, photo-cleavable linkers have been described in Kakiyama et al., “A peptide release system using a photo-cleavable linker in a cell array format for cell-toxicity analysis” Polymer Journal (2013) 45, 535-539; Baccile, J. A., et al., “Modular synthesis of photocleavable peptides using click chemistry.” Tetrahedron Letters volume 53, Issue 15, 11 Apr. 2012, p. 1933-1935; and Olejnik J. et al., “Photocleavable biotin phosphoramidite for 5′-end-labeling, affinity purification and phosphorylation of synthetic oligonucleotides.” Nucleic Acids Res. 1996 Jan. 15; 24(2):361-6; the entire contents of each are incorporated herein by reference. It should be appreciated, however, that additional photo-cleavable linkers would be apparent to the skilled artisan and are within the scope of this disclosure.

In some embodiments, the RNA binding protein is associated with the ARRDC1 protein, or variant thereof, via a sortase or transpeptidation reaction, and the linker comprises an LPXTG (e.g., for S. aureus sortase A), or LPXTA (e.g., for S. pyogenes sortase A) motif, where “X” represents any amino acid. A sortase refers to a group of prokaryotic enzymes that modify surface proteins by recognizing and cleaving a carboxyl-terminal sorting signal, for example, a sorting signal comprising the motif LPXTG or LPXTA. It should be appreciated, however, that additional sortase sorting signals would be recognized by the skilled artisan and are within the scope of this disclosure. Methods and reagents for conjugating proteins (e.g., an RNA binding protein and an ARRDC1 protein) using a sortase are known in the art and have been described previously, for example, in Levary, “Protein-Protein Fusion Catalyzed by Sortase A.” PLOS One, 2011 6(4): e18342; and Theile et al., “Site-specific N-terminal labeling of proteins using sortase-mediated reactions.” Nature Protocols. (2013) 8, 1800-1807; the entire contents of each are incorporated herein by reference. Accordingly, suitable methods for conjugating proteins as well as RNA binding proteins fused to an ARRDC1 protein, or variant thereof, to be included in an ARMM will be apparent to those of skill in the art based on this disclosure and knowledge in the art.

Any of the linkers, described herein, may be fused to the C-terminus of the ARRDC1 protein, or variant thereof, and the N-terminus of the RNA binding protein, or variant thereof, thereby linking the ARRDC1 protein, or variant thereof, to the RNA binding protein or RNA binding protein variant. In other embodiments, the linker may be fused to the C-terminus of the RNA binding protein, or variant thereof, and the N-terminus of the ARRDC1 protein, or variant thereof.

Any of the fusion proteins or linkers provided herein may comprise one or more additional features. Exemplary features that may be present include, without limitation, target peptides and protein tags. In some embodiments, any of the fusion proteins or linkers provided herein comprise one or more target peptides. In some embodiments, the fusion protein or linker comprises 1, 2, 3, 4, 5, 6, 7, 8, 9, or 10 target peptides. A fusion protein or linker comprising more than one target peptide may comprise the same target peptide, or different target peptides. As used herein, a “target peptide” refers to a peptide sequence, typically from 3-70 amino acids in length, that directs the transport of a protein to a specific region in the cell, including the nucleus, mitochondria, endoplasmic reticulum, peroxisome, and plasma membrane, however additional target peptides that target proteins to other regions of the cell would be apparent to the skilled artisan and are within the scope of this disclosure. In some embodiments, the target peptide is a peptide that directs a protein (e.g., a RNA binding protein bound to a binding RNA) to the nucleus. In some embodiments, the target peptide is a nuclear localization sequence. In some embodiments, the target peptide comprises the amino acid sequence PPKKKRKV (SEQ ID NO: 109). In some embodiments, the target peptide is a peptide that directs the protein to the secretory pathway. In some embodiments, the target peptide is a peptide that directs a protein (e.g., a RNA binding protein bound to a binding RNA) to the plasma membrane or the endoplasmic reticulum. In some embodiments, the target peptide that directs a protein to the plasma membrane or the endoplasmic reticulum is fused to the N-terminus of any of the fusion proteins provided herein. In some embodiments, the target peptide comprises the amino acid sequence MMSFVSLLLVGILFWATEAEQLTKCEVFQ (SEQ ID NO: 110). In some embodiments, the target peptide is a peptide that directs a protein to be retained at the endoplasmic reticulum. In some embodiments, the target peptide that directs a protein to be retained at the endoplasmic reticulum is fused to the C-terminus of any of the fusion proteins provided herein. In some embodiments, the target peptide comprises the amino acid sequence KDEL (SEQ ID NO: 111). In some embodiments, the target peptide is a peptide that directs a protein to the mitochondrial matrix. In some embodiments, the target peptide that directs a protein to the mitochondrial matrix is fused to the N-terminus of any of the fusion proteins provided herein. In some embodiments, the target peptide comprises the amino acid sequence MLSLRQSIRFFLPATRTLCSSRYLL (SEQ ID NO: 112). In some embodiments, the target peptide is a peptide that directs a protein to a peroxisome. In some embodiments, the target peptide is a PTS1 signal. In some embodiments, the PTS1 signal comprises the amino acid sequence SKL. In some embodiments, the target peptide is a PTS2 signal. In some embodiments, the PTS2 signal comprises the amino acid sequence RLXXXXXHL (SEQ ID NO: 113), wherein X is any amino acid. It should be appreciated, however, that the target peptides provided herein are exemplary and additional target peptides are also within the scope of this disclosure.

In some embodiments, any of the fusion proteins or linkers provided herein comprise one or more nuclear localization sequence (NLS). As used herein, a nuclear localization sequence refers to an amino acid sequence that promotes localization of a protein, for example, an RNA binding protein bound to a binding RNA having an NLS, into the nucleus of the cell (e.g., via nuclear transport). Typically, an NLS comprises one or more short amino acid sequences of positively charged lysines or arginines exposed on the protein surface. Nuclear localization sequences are known in the art and would be apparent to those skilled artisan. For example, nuclear localization sequences have been described in Kosugi et al., “Six Classes of Nuclear Localization Signals Specific to Different Binding Grooves of Importin α” J. Biol. Chem. Jan. 2, 2008, 284 p. 478-85; Kalderon et al., “A short amino acid sequence able to specify nuclear location” Cell (1984) 39 (3 Pt 2): 499-509; Dingwall et al., “The nucleoplasmin nuclear location sequence is larger and more complex than that of SV-40 large T antigen”. J Cell Biol. (1988) 107 (3): 841-9; Makkerh, et al., “Comparative mutagenesis of nuclear localization signals reveals the importance of neutral and acidic amino acids”. Curr Biol. (1996) 6 (8): 1025-7; and Ray et al., “Quantitative tracking of protein trafficking to the nucleus using cytosolic protein delivery by nanoparticle-stabilized nanocapsules”. Bioconjug. Chem. (2015) 26 (6): 1004-7; the entire contents of each of which are incorporated herein by reference. Additional nuclear localization sequences are described, for example, in Plank et al., international PCT application, PCT/EP2000/011690, the entire contents of which are incorporated herein by reference. In some embodiments, a NLS comprises the amino acid sequence PKKKRKV (SEQ ID NO: 114) or MDSLLMNRRKFLYQFKNVRWAKGRRETYLC (SEQ ID NO: 115).

In some embodiments, the RNA binding protein is fused to at least one NLS. In some embodiments, one or more nuclear localization sequences (NLSs) are fused to the N-terminus of an RNA binding protein. In some embodiments, one or more NLSs are fused to the C-terminus of an RNA binding protein. In some embodiments, an RNA binding protein is fused to at least one, at least two, at least three, at least four, at least five, at least six, at least seven, at least eight, at least nine, at least ten, or more NLSs. It should be appreciated that one or more NLSs may be fused to an RNA binding protein to allow localization of the RNA binding protein into the nucleus of a target cell. In some embodiments, the RNA binding protein fused to at least one NLS is associated with ARRDC1, or an ARRDC1 protein variant.

In some embodiments, any of the fusion proteins or linkers provided herein comprise one or more protein tags, which may be useful for solubilization, purification, or detection of the fusion proteins. In some embodiments, the fusion protein or linker comprises 1, 2, 3, 4, 5, 6, 7, 8, 9, or 10 protein tags. Suitable protein tags are provided herein, and include, without limitation, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable protein tags will be apparent to those of skill in the art and are within the scope of this disclosure.

### WW Domain Containing RNA Binding Proteins

Aspects of the disclosure relate to ARMMs comprising an RNA binding protein associated with at least one WW domain (e.g., WW:Tat). In some aspects, fusion proteins are provided that comprise an RNA binding protein with at least one WW domain. In some aspects, expression constructs are provided that encode an RNA binding protein associated with at least one WW domain. The WW domain of a cargo protein may associate with the PPXY (SEQ ID NO: 2) motif of the ARRDC1 protein, or variant thereof, to facilitate association with or inclusion of the RNA binding protein into an ARMM. A schematic representation of a Tat RNA binding protein fused to a WW domain that associates with the PPXY (SEQ ID NO: 2) motif of ARRDC1 can be seen in FIG. 1B. In some embodiments, the RNA binding protein is fused to at least one, at least two, at least three, at least four, at least five, at least six, at least seven, at least eight, at least nine, at least ten, or more WW domains. The WW domain may be derived from a WW domain of ubiquitin ligase WWP1, WWP2, Nedd4-1, Nedd4-2, Smurf1, Smurf2, ITCH, NEDL1, or NEDL2 (FIG. 3). For example, the WW domain may comprise a WW domain or WW domain variant from the amino acid sequence set forth in (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); or (SEQ ID NO: 14). In certain embodiments, the RNA binding proteins may comprise two WW domains, or WW domain variants, from the human ITCH protein having the amino acid sequence:

(SEQ ID NO: 18)
PLPPGWEQRVDQHGRVYYVDHVEKRTTWDRPEPLPPGWERRVDNMGRIYY
VDHFTRTTTWQRPTL.

In other embodiments, RNA binding proteins may comprise four WW domains, or WW domain variants, from the human ITCH protein having the amino acid sequence:

(SEQ ID NO: 19)
PLPPGWEQRVDQHGRVYYVDHVEKRTTWDRPEPLPPGWERRVDNMGRIYY
VDHFTRTTTWQRPTLESVRNYEQWQLQRSQLQGAMQQFNQRFIYGNQDLF
ATSQSKEFDPLGPLPPGWEKRTDSNGRVYFVNHNTRITQWEDPRSQGQLN
EKPLPEGWEMRFTVDGIPYFVDHNRRTTTYIDPRT.

The RNA binding proteins, described herein, that are fused to at least one WW domain or WW domain variant are non-naturally occurring, that is, they do not exist in nature.

In some embodiments, one or more WW domains may be fused to the N-terminus of an RNA binding protein. In other embodiments, one or more WW domains may be fused to the C-terminus of an RNA binding protein. In yet other embodiments, one or more WW domains may be inserted into an RNA binding protein. It should be appreciated that the WW domains may be configured in any number of ways to maintain function of the RNA binding protein, which can be tested by methods known to one of ordinary skill in the art. In some embodiments, at least one WW domain is fused to the N-terminus of an RNA binding protein. In some embodiments, 1, 2, 3, 4, 5, 6, 7, 8, 9, or 10 WW domains are fused to the N-terminus of an RNA binding protein. In some embodiments, at least one WW domain is fused to the C-terminus of an RNA binding protein. In some embodiments, 1, 2, 3, 4, 5, 6, 7, 8, 9, or 10 WW domains are fused to the C-terminus of an RNA binding protein.

The RNA binding protein of the inventive microvesicles may be a protein comprising at least one WW domain. For example, the RNA binding protein may be a WW domain containing protein or a protein fused to at least one WW domain. In some embodiments, the RNA binding protein may be a Tat protein or Tat protein variant fused to at least one WW domain.

### RNA Binding Proteins

Some aspects of the disclosure relate to proteins that bind to RNA. In some embodiments, the RNA binding protein is a naturally-occurring protein, or non-naturally-occurring variant thereof, or a non-naturally occurring protein that binds to an RNA, for example, an RNA with a specific sequence or structure.

In certain embodiments, the RNA binding protein is a trans-activator of transcription (Tat) protein that specifically binds a trans-activating response element (TAR element). An exemplary Tat protein comprises the amino acid sequence as set forth in SEQ ID NO: 65 (Table 1). Exemplary amino acid sequences of Tat proteins, as well as Tat protein fragments that bind TAR elements, are shown in Table 1. In some embodiments, the RNA binding protein is at least 60%, 65%, 70%, 75%, 80%, 85%, 90%, 95%, 98%, or 99% identical to the amino acid sequence of any one of SEQ ID NOs: 65-84, and binds a TAR element. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, or at least 130 identical contiguous amino acids of any one of SEQ ID NOs: 65-84, and binds a TAR element. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 65-84, and binds a TAR element. In some embodiments, the RNA binding protein comprises any one of the amino acid sequences set forth in SEQ ID NOs: 65-84. In some embodiments, the Tat protein comprises an amino acid sequence as set forth in any one of SEQ ID NOs: 65-84. The RNA binding protein may also be a variant of a Tat protein that is capable of associating with a TAR element. Tat proteins, as well as variants of Tat proteins that bind to a TAR element, are known in the art and have been described previously, for example, in Kamine et al., “Mapping of HIV-1 Tat Protein Sequences Required for Binding to Tar RNA”, Virology 182, 570-577 (1991); and Patel, “Adaptive recognition in RNA complexes with peptides and protein modules” Curr Opin Struct Biol. 1999 February; 9(1):74-87; the entire contents of each of which are incorporated herein by reference. In some embodiments, the Tat protein is an HIV-1 Tat protein, or variant thereof. In some embodiments, the Tat protein is bovine immunodeficiency virus (BIV) Tat protein, or variant thereof.

A Tat protein is a nuclear transcriptional activator of viral gene expression that is essential for viral transcription from the LTR promoter and replication; it acts as a sequence-specific molecular adapter, directing components of the cellular transcription machinery to the viral RNA to promote processive transcription elongation by the RNA polymerase II (RNA pol II) complex, thereby increasing the level of full-length transcripts. Tat binds to a hairpin structure at the 5′-end of all nascent viral mRNAs referred to as the transactivation responsive RNA element (TAR RNA) in a CCNT1-independent mode.

The Tat protein consists of several domains, one is a short lysine and arginine rich region important for nuclear localization. The nine amino acid basic region of HIV-1 Tat is found at positions 49-57 of SEQ ID NO: 65, and is capable of binding a TAR element. In some embodiments, the Tat sequence comprises the nine amino acid basic region of Tat (SEQ ID NO: 73). In some embodiments the RNA binding protein comprises any one of the amino acid sequences as set forth in SEQ ID NOs: 65-67, 69, 70, or 73-84. In some embodiments, the Tat proteins are fusion proteins.

TABLE 1
Tat Sequences

| Tat (Residue NOs) | Sequence | SEQ ID NO |
|---|---|---|
| HIV-1 Tat (1-101) | MEPVDPRLEPWKHPGSQPRT PCTTCYCKKC | 65 |
| | CFHCQVCFTT KALGISYGRK KRRQRRRPPQ | |
| | GSQTHQVSLS KQPSSQPRGD QTGPKESKKK | |
| | VERETEADPKP | |
| HIV-1 Tat (1-86) | MEPVDPRLEP WKHPGSQPRT PCTTCYCKKC | 66 |
| | CFHCQVCFTT KALGISYGRK KRRQRRRPPQ | |
| | GSQTHQVSLS KQPSSQPRGD QTGPKE | |
| HIV-1 Tat (37-72) | CFTT KALGISYGRK KRRQRRRPPQ GSQTHQVSLS | 67 |
| | KQ | |
| HIV-1 Tat (1-45) | MEPVDPRLEP WKHPGSQPRT PCTTCYCKKC | 68 |
| | CFHCQVCFTT KALGI | |
| HIV-1 Tat (49-86) | RK KRRQRRRPPQ GSQTHQVSLS KQPSSQPRGD | 69 |
| | QTGPKE | |
| HIV-1 Tat (52-86) | RRQRRRPPQ GSQTHQVSLS KQPSSQPRGD QTGPKE | 70 |
| HIV-1 Tat (55-86) | RRRPPQ GSQTHQVSLS KQPSSQPRGD QTGPKE | 71 |
| HIV-1 Tat (58-86) | PPQ GSQTHQVSLS KQPSSQPRGD QTGPKE | 72 |
| HIV-1 Tat (49-57) | RK KRRQRRR | 73 |
| HIV-1 Tat (49-59) | RK KRRQRRRPP | 74 |
| HIV-1 Tat (49-61) | RK KRRQRRRPPQ G | 75 |
| HIV-1 Tat (49-63) | RK KRRQRRRPPQ GSQ | 76 |
| HIV-1 Tat (49-65) | RK KRRQRRRPPQ GSQTH | 77 |
| HIV-1 Tat (37-57) | CFTT KALGISYGRK KRRQRRR | 78 |
| HIV-1 Tat (38-62) | CFTT KALGISYGRK KRRQRRRPPQ GSQ | 79 |
| HIV-1 Tat (47-58) | GRRK KRRQRRRP | 80 |
| HIV-1 Tat (46-65) | RK KRRQRRRPPQ GSQTH | 81 |
| HIV-2 Tat (1-130) | METPLKAPEG SLGSYNEPSS CTSEQDAAAQ | 82 |
| | GLVSPGDEIL YQLYQPLEAC DNKCYCKKCC | |
| | YHCQMCFLNK GLGIWYERKG RRRRTPKKTK | |
| | AHSSSASDKS ISTRTGNSQP EKKQKKTLET | |
| | ALETIGGPGR | |
| BIV Tat | MPGPWVAMIM LPQPKESFGG KPIGWLFWNT | 83 |
| | CKGPRRDCPH CCCPICSWHC QLCFLQKNLG | |
| | INYGSGPRRR GTRGKGRRIR RTASGGDQRR | |
| | EADSQRSFTN MDQ | |
| BIV Tat | SGPRPRGTRGKGRRIRR | 84 |

In some embodiments, the RNA binding protein is a regulator of virion expression (Rev) protein (e.g., Rev from HIV-1), or variant thereof, that binds to a Rev response element (RRE). Rev proteins are known in the art and are known to the skilled artisan. For example, Rev proteins have been described in Fernandes et al., “The HIV-1 Rev response element: An RNA scaffold that directs the cooperative assembly of a homo-oligomeric ribonucleoprotein complex” RNA Biology 9:1, 6-11; January 2012; Cochrane et al., “The human immunodeficiency virus Rev protein is a nuclear phosphoprotein” Virology 171 (1):264-266, 1989; Grate et al., “Role REVersal: understanding how RRE RNA binds its peptide ligand” Structure. 1997 Jan. 15; 5(1):7-11; and Patel, “Adaptive recognition in RNA complexes with peptides and protein modules” Curr Opin Struct Biol. 1999 February; 9(1):74-87; the entire contents of each of which are incorporated herein by reference in their entirety. An exemplary Rev protein comprises the amino acid sequence as set forth in SEQ ID NOs: 93-95 (Table 3). In some embodiments, the RNA binding protein is at least 60%, 65%, 70%, 75%, 80%, 85%, 90%, 95%, 98%, or 99% identical to the amino acid sequence of any one of SEQ ID NOs: 93-95, and binds a Rev response element. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or at least 115 identical contiguous amino acids of any one of SEQ ID NOs: 93-95, and binds a Rev response element. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 93-95, and binds a Rev response element. In some embodiments, the RNA binding protein comprises any one of the amino acid sequences set forth in SEQ ID NOs: 93-95. In some embodiments, the RNA binding protein comprises a variant of any one of the amino acid sequences as set forth in SEQ ID NOs: 93-95 that are capable of binding an RRE. Such variants would be apparent to the skilled artisan based on this disclosure and knowledge in the art and may be tested (e.g. for binding to an RRE) using routine methods known in the art.

In some embodiments, the RNA binding protein is a coat protein of an MS2 bacteriophage that specifically binds to an MS2 RNA. MS2 bacteriophage coat proteins that specifically bind MS2 RNAs are known in the art. For example MS2 phage coat proteins have been described in Parrott et al., “RNA aptamers for the MS2 bacteriophage coat protein and the wild-type RNA operator have similar solution behavior” Nucl. Acids Res. 28(2):489-497 (2000); Keryer-Bibens et al., “Tethering of proteins to RNAs by bacteriophage proteins” Biol. Cell. 100(2): 125-38 (2008); and Patel, “Adaptive recognition in RNA complexes with peptides and protein modules” Curr Opin Struct Biol. 1999 February; 9(1):74-87; the entire contents of each are hereby incorporated by reference in their entirety. An exemplary MS2 phage coat protein comprises the amino acid sequence as set forth in SEQ ID NO: 99 (Table 4). In some embodiments, the RNA binding protein is at least 60%, 65%, 70%, 75%, 80%, 85%, 90%, 95%, 98%, or 99% identical to the amino acid sequence of SEQ ID NO: 99, and binds an MS2 RNA. In some embodiments, the RNA binding protein has at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, or at least 115 identical contiguous amino acids of SEQ ID NO: 99, and binds an MS2 RNA. In some embodiments, the RNA binding protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to SEQ ID NO: 99, and binds an MS2 RNA. In some embodiments, the RNA binding protein comprises the amino acid sequence set forth in SEQ ID NO: 99. In some embodiments, the RNA binding protein comprises a fragment or variant of SEQ ID NO: 99 that is capable of binding to an MS2 RNA. Methods for testing whether variants or fragments of MS2 phage coat proteins bind to MS2 RNAs (e.g., SEQ ID NO: 99) can be performed using routine experimentation and would be apparent to the skilled artisan.

In some embodiments, the RNA binding protein is a P22 N protein (e.g., P22 N from bacteriophage), or variant thereof, that binds to a P22 boxB RNA. P22 N proteins are known in the art and would be apparent to the skilled artisan. For example, P22 N proteins have been described in Cai et al., “Solution structure of P22 transcriptional antitermination N peptide-boxB RNA complex” Nat Struct Biol. 1998 March; 5(3):203-12; and Patel, “Adaptive recognition in RNA complexes with peptides and protein modules” Curr Opin Struct Biol. 1999 February; 9(1):74-87; the entire contents of each are incorporated by reference herein. An exemplary P22 N that specifically binds to a protein P22 boxB RNA comprises the amino acid sequence NAKTRRHERRRKLAIERDTI (SEQ ID NO: 100).

In some embodiments, the RNA binding protein is a λ N protein (e.g., λ N from bacteriophage), or variant thereof, that binds to a λ boxB RNA. λ N proteins are known in the art and would be apparent to the skilled artisan. For example, λ N proteins have been described in Keryer-Bibens et al., “Tethering of proteins to RNAs by bacteriophage proteins” Biol Cell. 2008 February; 100(2):125-38; Legault et al., “NMR structure of the bacteriophage lambda N peptide/boxB RNA complex: recognition of a GNRA fold by an arginine-rich motif” Cell. 1998 Apr. 17; 93(2):289-99; and Patel, “Adaptive recognition in RNA complexes with peptides and protein modules” Curr Opin Struct Biol. 1999 February; 9(1):74-87; the entire contents of each are incorporated by reference herein. An exemplary λ N protein that specifically binds to a λ boxB comprises the amino acid sequence GSMDAQTRRRERRAEKQAQWKAAN (SEQ ID NO: 101).

In some embodiments, the RNA binding protein is a φ21 N protein (e.g., φ21 N from bacteriophage), or variant thereof, that binds to a φ21 boxB RNA. φ21 N proteins are known in the art and would be apparent to the skilled artisan. For example, φ21 proteins have been described in Cilley et al. “Structural mimicry in the phage φ21 N peptide-boxB RNA complex.” RNA. 2003; 9(6):663-676; and Patel, “Adaptive recognition in RNA complexes with peptides and protein modules” Curr Opin Struct Biol. 1999 February; 9(1):74-87; the entire contents of each are incorporated by reference herein. An exemplary φ21 N protein that specifically binds to a φ21 boxB RNA comprises amino acid sequence GTAKSRYKARRAELIAERR (SEQ ID NO: 102). The N peptide binds as an α-helix and interacts predominately with the major groove side of the 5′ half of the boxB RNA stem-loop. This binding interface is defined by surface complementarity of polar and nonpolar interactions. The N peptide complexed with the exposed face of the φ21 boxB loop is similar to the GNRA tetraloop-like folds of the related λ and P22 bacteriophage N peptide-boxB RNA complexes.

In some embodiments, the RNA binding protein is a HIV-1 nucleocapsid (e.g., nucleocapsid from HIV-1), or variant thereof, that binds to a SL3 ψ RNA. HIV-1 nucleocapsid proteins are known in the art and would be apparent to the skilled artisan. For example, HIV-1 nucleocapsid proteins have been described in Patel, “Adaptive recognition in RNA complexes with peptides and protein modules” Curr Opin Struct Biol. 1999 February; 9(1):74-87; the entire contents of which is incorporated by reference herein. An exemplary HIV-1 nucleocapsid that specifically binds to a SL3 ψ RNA comprises amino acid sequence

(SEQ ID NO: 103)
MQKGNFRNQRKTVKCFNCGKEGHIAKNCRAPRKKGCWKCGKEGHQMKDCT
ERQAN.

### Binding RNAs

Some aspects of the disclosure relate to RNA molecules that bind proteins. In some embodiments, the binding RNA is a naturally occurring RNA, or non-naturally occurring variant thereof, or a non-naturally occurring RNA, that binds to a protein having a specific amino acid sequence or structure.

In certain embodiments, the binding RNA is a trans-activating response element (TAR element), which is an RNA stem-loop structure that is found at the 5′ ends of nascent human immunodeficiency virus-1 (HIV-1) transcripts and specifically bind to a trans-activator of transcription (Tat) protein. In some embodiments, the TAR element is a bovine immunodeficiency virus (BIV) TAR. An exemplary TAR element comprises the nucleic acid sequence as set forth in SEQ ID NO: 84. Further exemplary TAR sequences can be found in Table 2; however, these sequences are not meant to be limiting and additional TAR element sequences that bind to a Tat protein, or variant thereof, are also within the scope of this disclosure. The binding RNA may also be a variant of a TAR element that is capable of associating with the RNA binding protein, trans-activator of transcription (Tat protein), which is a regulatory protein that is involved in transcription of the viral genome. Variants of TAR elements that are capable of associating with Tat proteins would be apparent to the skilled artisan based on this disclosure and knowledge in the art, and are within the scope of this disclosure. Further, the association between a TAR variant and a Tat protein, or Tat protein variant, may be tested using routine methods. TAR elements and variants of TAR elements that bind to Tat proteins are known in the art and have been described previously, for example in Kamine et al., “Mapping of HIV-1 Tat Protein Sequences Required for Binding to Tar RNA” Virology 182, 570-577 (1991); and Patel, “Adaptive recognition in RNA complexes with peptides and protein modules” Curr Opin Struct Biol. 1999 February; 9(1):74-87; the entire contents of each are incorporated by reference herein. In some embodiments, the binding RNA comprises the nucleic acid sequence as set forth in SEQ ID NOs: 85-90. In some embodiments, the binding RNA comprises a variant of any of the nucleic acid sequences set forth in SEQ ID NOs: 85-90 that are capable of binding to a Tat protein or variant thereof.

Without wishing to be bound by any particular theory, a TAR element is capable of forming a stable stem-loop structure (Muesing et al., 1987) in the native viral RNA. On the stem of TAR, a three nucleotide bulge, has been demonstrated to play a role in high-affinity binding of the Tat protein to the TAR element (Roy et al., 1990; Cordingley et al., 1990; Dingwall et al., 1989; Weeks et al., 1990). In the TAR element, the integrity of the stem and the initial U22 of the bulge may contribute to Tat protein binding (Roy et al., 1990b). Other sequences that may not affect the binding of the Tat protein to the TAR site play a role in trans-activation of transcription in vivo. One such region is the sequence at the loop, which is required for the binding of cellular factors that may interact with the Tat protein to mediate transactivation (Gatignol et al., 1989; Gaynor et al., 1989; Marciniak et al., 1990a; Gatignol et al., 1991).

TABLE 2
TAR Sequences

| TAR | Sequence | SEQ ID NO |
|---|---|---|
| HIV- 1 TAR RNA + | gggucucucugguuagaccagaucugagccugggagcucucuggcuaa | 85 |
| 1-59 | cuagggaacccacug | |
| Δ TAR | gggucucucugguuagaccagaucugagccugggcucuggcuaacuag | 86 |
| | ggaacccacug | |
| HIV- 1TAR | gggucucucugguuagaccagaucugagccugggagcucucuggcuaa | 87 |
| (shown in FIG. | cuagggaacc | |
| 2) | | |
| HIV- 1 TAR | agaucugagccugggagcucucu | 88 |
| Hybrid TAR | gcucguugagcucugggaagcuccgagc | 89 |
| BIV TAR | ucguguagcucauuagcuccga | 90 |

In some embodiments, the binding RNA is a Rev response element (RRE), or variant thereof, that binds to a Rev protein (e.g., Rev from HIV-1). Rev response elements are known in the art and would be apparent to the skilled artisan for use in the present invention. For example, Rev response elements have been described in Fernandes et al., “The HIV-1 Rev response element: An RNA scaffold that directs the cooperative assembly of a homo-oligomeric ribonucleoprotein complex.” RNA Biology 9:1, 6-11, January 2012; Cook et al., “Characterization of HIV-1 REV protein: binding stoichiometry and minimal RNA substrate.” Nucleic Acids Res. April 11; 19(7):1577-1583, 1991; Grate et al., “Role REVersal: understanding how RRE RNA binds its peptide ligand” Structure. 1997 Jan. 15; 5(1):7-11; and Patel, “Adaptive recognition in RNA complexes with peptides and protein modules” Curr Opin Struct Biol. 1999 February; 9(1):74-87; the entire contents of each are incorporated herein by reference. Any of the RRE nucleic acid sequences or any of the fragments of RRE nucleic acid sequences described in the above references may be used as binding RNAs in accordance with this disclosure. Exemplary RRE nucleic acid sequences that bind Rev include, without limitation, those nucleic acid sequences set forth in SEQ ID NOs: 91 and 92 (Table 3).

In some embodiments, the Rev peptide may adopt a particular structure and several amino acids, rather than a single arginine, may participate in sequence-specific RNA interactions. Without wishing to be bound by any particular theory, Rev recognition of the RRE, like Tat recognition of TAR, is due to direct binding. Binding can be tight (Kd=1-3 nM) and highly specific for the RRE. As the concentration of Rev increases, progressively larger complexes with RRE RNA are formed, whereas Tat forms one-to-one complexes with TAR RNA.

Generally, a Rev protein may bind initially to a high affinity site and subsequently additional Rev molecules occupy lower affinity sites. RNAs that bind Rev have been described in Heaphy et al., “HIV-1 regulator of virion expression (Rev) protein binds to an RNA stem-loop structure located within the Rev-response element region” Cell, 1990. 60, 685-693; the entire contents of which is incorporated by reference herein.

TABLE 3
RRE/Rev Sequences

| | Sequence | SEQ ID NO |
|---|---|---|
| HIV-1 RRE | ggucugggcgcagcgcaagcugacgguacaggcc | 91 |
| HIV-1 RRE | ggcuggacucguacuucgguacuggagaaacagcc | 92 |
| aptamer | | |
| HIV-1 Rev | MAGRSGDSDEELIRTVRLIKLLYQSNPPPNPEGTRQ | 93 |
| | ARRNRRRRWRERQRQIHSISERILGTYLGRSAEPVP | |
| | LQLPPLERLTLDCNEDCGTSGTQGVGSPQILVESPT | |
| | VLESGTKE | |
| HIV-1 Rev peptide | TRQARRNRRRRWRERQR | 94 |
| Evolved HIV-1 | RDRRRRGSRPSGAERRRRRAAAA | 95 |
| RRE-binding | | |
| peptide | | |

In some embodiments, the binding RNA is an MS2 RNA that specifically binds to a MS2 phage coat protein. Typically, the coat protein of the RNA bacteriophage MS2 binds a specific stem-loop structure in viral RNA (e.g., MS2 RNA) to accomplish encapsidation of the genome and translational repression of replicase synthesis. RNAs that specifically bind MS2 phage coat proteins are known in the art and would be apparent the skilled artisan. For example RNAs that bind MS2 phage coat proteins have been described in Parrott et al., “RNA aptamers for the MS2 bacteriophage coat protein and the wild-type RNA operator have similar solution behavior.” Nucl. Acids Res. 28(2): 489-497 (2000); Witherell et al., “Specific interaction between RNA phage coat proteins and RNA.” Prog Nucleic Acid Res Mol Biol. 1991; 40:185-220; Stockley et al., “Probing sequence-specific RNA recognition by the bacteriophage MS2

coat protein.” Nucleic Acids Res. 1995 Jul. 11; 23(13):2512-8; Keryer-Bibens C., et al., “Tethering of proteins to RNAs by bacteriophage proteins.” Biol. Cell. 100(2): 125-38 (2008); and Patel. “Adaptive recognition in RNA complexes with peptides and protein modules.” Curr Opin Struct Biol. 1999 February; 9(1):74-87; the entire contents of each are hereby incorporated by reference in their entirety. In some embodiments, an exemplary MS2 RNA that specifically binds to a MS2 phage coat protein comprises a nucleic acid sequence as set forth in any one of SEQ ID NOs: 96-98 (Table 4). In some embodiments, the binding RNA comprises the nucleic acid sequence of any one of SEQ ID NOs: 96, 97, or 98.

TABLE 4
MS2 Sequences

| MS2 | Sequence | SEQ ID NO |
|---|---|---|
| Bacteriophage | acaugaggauuacccaugu | 96 |
| MS2 RNA | | |
| MS2 RNA | ccggaggaucaccacggg | 97 |
| MS2 RNA | ccacagucacuggg | 98 |
| Bacteriophage | ASNFTQFVLVDNGGTGDVTVAPSNFANGVAEWIS | 99 |
| MS2 Coat Protein | SNSRSQAYKVTCSVRQSSAQNRKYTIKVEVPKVAT | |
| | QTVGGVELPVAAWRSYLNMELTIPIFATNSDCELI | |
| | VKAMQ GLLKDGNPIP SAIAANSGIY | |

In some embodiments, the binding RNA is an RNA that specifically binds to a P22 N protein (e.g., P22 N from bacteriophage), or variant thereof. P22 N proteins are known in the art and would be apparent to the skilled artisan. For example, P22 N proteins have been described in Cai et al., “Solution structure of P22 transcriptional antitermination N peptide-boxB RNA complex” Nat Struct Biol. 1998 March; 5(3):203-12; Weiss, “RNA-mediated signaling in transcription” Nat Struct Biol. 1998 May; 5(5):329-33; and Patel, “Adaptive recognition in RNA complexes with peptides and protein modules” Curr Opin Struct Biol. 1999 February; 9(1):74-87; the entire contents of each are incorporated by reference herein. An exemplary P22 boxB RNA that specifically binds to a P22 N protein comprises a nucleic acid sequence as set forth in gcgcugacaaagcgc (SEQ ID NO: 104).

In some embodiments, the binding RNA is an RNA that specifically binds to a λ N protein (e.g., λ N from bacteriophage), or variant thereof. λ N proteins are known in the art and would be apparent to the skilled artisan. For example, λ N proteins have been described in Keryer-Bibens et al., “Tethering of proteins to RNAs by bacteriophage proteins.” Biol Cell. 2008 February; 100(2):125-38; Weiss. “RNA-mediated signaling in transcription.” Nat Struct Biol. 1998 May; 5(5):329-33; Legault et al., “NMR structure of the bacteriophage lambda N peptide/boxB RNA complex: recognition of a GNRA fold by an arginine-rich motif.” Cell. 1998 Apr. 17; 93(2):289-99; and Patel, “Adaptive recognition in RNA complexes with peptides and protein modules.” Curr Opin Struct Biol. 1999 February; 9(1):74-87; the entire contents of each are incorporated by reference herein. An exemplary λ boxB RNA that specifically binds to a λ N protein comprises a nucleic acid sequence as set forth in gggcccugaagaagggccc (SEQ ID NO: 105).

In some embodiments, the binding RNA is an RNA that specifically binds to a φ21 N protein (e.g., φ21 N from bacteriophage), or variant thereof. φ21 N proteins are known in the art and would be apparent to the skilled artisan. For example, φ21 proteins have been described in Cilley et al. “Structural mimicry in the phage φ21 N peptide-boxB RNA complex.” RNA. 2003; 9(6):663-676; and Patel, “Adaptive recognition in RNA complexes with peptides and protein modules.” Curr Opin Struct Biol. 1999 February; 9(1):74-87; the entire contents of each are incorporated by reference herein. An exemplary φ21 boxB RNA that specifically binds to φ21 N protein comprises a nucleic acid sequence as set forth in ucucaaccuaaccguugaga (SEQ ID NO: 106).

In some embodiments, the binding RNA is an RNA that specifically binds to an HIV-1 nucleocapsid protein (e.g., nucleocapsid from HIV-1) or variant thereof. HIV-1 nucleocapsid proteins are known in the art and would be apparent to the skilled artisan. For example, HIV-1 nucleocapsid proteins have been described in Patel, “Adaptive recognition in RNA complexes with peptides and protein modules.” Curr Opin Struct Biol. 1999 February; 9(1):74-87; the entire contents of which is incorporated by reference herein. An exemplary SL3 ψ RNA that specifically binds to a HIV-1 nucleocapsid comprises a nucleic acid sequence as set forth in ggacuagcggaggcuagucc (SEQ ID NO: 107).

It should be appreciated that the binding RNAs of the present disclosure need not be limited to naturally-occurring RNAs or non-naturally-occurring variants thereof, that have recognized protein binding partners. In some embodiments, the binding RNA may also be a synthetically produced RNA, for example an RNA that is designed to specifically bind to a protein (e.g., an RNA binding protein). In some embodiments, the binding RNA is designed to specifically bind to any protein of interest, for example ARRDC1. In some embodiments, the binding RNA is an RNA produced by the systematic evolution of ligands by exponential enrichment (SELEX). SELEX methodology would be apparent to the skilled artisan and has been described previously, for example in U.S. Pat. Nos. 5,270,163; 5,817,785; 5,595,887; 5,496,938; 5,475,096; 5,861,254; 5,958,691; 5,962,219; 6,013,443; 6,030,776; 6,083,696; 6,110,900; 6,127,119; and 6,147,204; U.S. Appln 20030175703 and 20030083294, Potti et al., Expert Opin. Biol. Ther. 4:1641-1647 (2004), and Nimjee et al., Annu. Rev. Med. 56:555-83 (2005). The technique of SELEX has been used to evolve aptamers to have extremely high binding affinity to a variety of target proteins. See, for example, Trujillo U. H., et al., “DNA and RNA aptamers: from tools for basic research towards therapeutic applications”. Comb Chem High Throughput Screen 9 (8): 619-32 (2006) for its disclosure of using SELEX to design aptamers that bind vascular endothelial growth factor (VEGF). In some embodiments, the binding RNA is an aptamer that specifically binds a target protein, for example a protein found in an ARMM (e.g., ARRDC1 or TSG101).

### Cargo RNAs

Some aspects of the disclosure provide RNAs that are associated with, for example, incorporated into the liquid phase of, an ARMM. In some embodiments, a cargo RNAis an RNA molecule that can be delivered via its association with or inclusion in an ARMM to a subject, organ, tissue, or cell. In some embodiments, the cargo RNA is to be delivered to a target cell in vitro, in vivo, or ex vivo. In some embodiments, the cargo RNA to be delivered is a biologically active agent, i.e., it has activity in a cell, organ, tissue, and/or subject. For instance, an RNA that, when administered to a subject, has a biological effect on that subject, or is considered to be biologically active. In certain embodiments the cargo RNA is a messenger RNA or an RNA that expresses a protein in a cell. In certain embodiments, the cargo RNA is a small interfering RNA (siRNA) that inhibits the expression of one or more genes in a cell. In some embodiments, a cargo RNA to be delivered is a therapeutic agent, for example, an agent that has a beneficial effect on a subject when administered to a subject. In some embodiments, the cargo RNA to be delivered to a cell is an RNA that expresses a transcription factor, a tumor suppressor, a developmental regulator, a growth factor, a metastasis suppressor, a pro-apoptotic protein, a nuclease, or a recombinase. In some embodiments, the cargo RNA to be delivered is an RNA that expresses p53, Rb (retinoblastoma protein), a BIM protein, BRCA1, BRCA2, PTEN, adenomatous polyposis coli (APC), CDKN1B, cyclin-dependent kinase inhibitor 1C, HEPACAM, INK4, Mir-145, p16, p63, p73, SDHB, SDHD, secreted frizzled-related protein 1, TCF21, TIG1, TP53, tuberous sclerosis complex tumor suppressors, Von Hippel-Lindau (VHL) tumor suppressor, CD95, ST7, ST14, a BCL-2 family protein, a caspase; BRMS1, CRSP3, DRG1, KAI1, KISS1, NM23, a TIMP-family protein, a BMP-family growth factor, EGF, EPO, FGF, G-CSF, GM-CSF, a GDF-family growth factor, HGF, HDGF, IGF, PDGF, TPO, TGF-α, TGF-β, VEGF; a zinc finger nuclease, Cre, Dre, or FLP recombinase.

In some embodiments, the cargo RNA may be an RNA that inhibits expression of one or more genes in a cell. For example, in some embodiments, the cargo RNA is a microRNA (miRNA), a small interfering RNA (siRNA) or an antisense RNA (asRNA).

In some embodiments, the cargo RNA to be delivered comprises a messenger RNA (mRNA), a ribosomal RNA (rRNA), a signal recognition particle RNA (SRP RNA), or a transfer RNA (tRNA). In some embodiments, the cargo RNA to be delivered comprises a small nuclear RNA (snRNA), a small nucleolar (snoRNA), a SmY RNA (smY), a guide RNA (gRNA), a ribonuclease P (RNase P), a ribonuclease MRP (RNase MRP), a Y RNA, a telomerase RNA component (TERC), or a spliced leader RNA (SL RNA). In some embodiments, the cargo RNA to be delivered comprises an antisense RNA (asRNA), a cis-natural antisense sequence (cis-NAT), a CRISPR RNA (crRNA), a long noncoding RNA (lncRNA), a microRNA (miRNA), a piwi-interacting RNA (piRNA), a small interfering RNA (siRNA), or a trans-acting siRNA (tasiRNA).

In some embodiments, the cargo RNA to be delivered is a diagnostic agent. In some embodiments, the cargo RNA to be delivered is a prophylactic agent. In some embodiments, the cargo RNA to be delivered is useful as an imaging agent. In some of these embodiments, the diagnostic or imaging agent is, and in others it is not, biologically active.

In some embodiments, any of the cargo RNAs provided herein are associated with a binding RNA. In some embodiments, the cargo RNA is covalently associated with the binding RNA. In some embodiments, the cargo RNA and the binding RNA are part of the same RNA molecule, (e.g., an RNA from a single transcript). In some embodiments, the cargo RNA and the binding RNA are covalently associated via a linker. In some embodiments, the linker comprises a nucleotide or nucleic acid (e.g., DNA or RNA). In some embodiments, the linker comprises RNA. In some embodiments, the linker comprises at least 1, at least 2, at least 3, at least 4, at least 5, at least 6, at least 7, at least 8, at least 9, at least 10, at least 15, at least 20, at least 25, at least 30, at least 40, at least 50, at least 100, at least 150, at least 200, at least 250, at least 300, at least 400, or at least 500 nucleotides (e.g., DNA or RNA).

In other embodiments, the cargo RNA is non-covalently associated with the binding RNA. For example, the cargo RNA may associate with the binding RNA via complementary base pairing. In some embodiments, the cargo RNA is bound to the binding RNA via at least 2, at least 3, at least 4, at least 5, at least 6, at least 7, at least 8, at least 9, at least 10, at least 15, at least 20, at least 25, at least 30, at least 40, at least 50, complementary base pairs, which may be contiguous or non-contiguous. In some embodiments, the cargo RNA is bound to the binding RNA via at least 2, at least 3, at least 4, at least 5, at least 6, at least 7, at least 8, at least 9, at least 10, at least 15, at least 20, at least 25, at least 30, at least 40, at least 50 contiguous complementary base pairs.

It should be appreciated that any of the RNAs provided herein (e.g., binding RNAs, cargo RNAs, and/or binding RNAs fused to cargo RNAs) may comprise one or more modified oligonucleotides. In some embodiments, any of the RNAs described herein may be modified, e.g., comprise a modified sugar moiety, a modified internucleoside linkage, a modified nucleotide and/or combinations thereof. In some embodiments, RNA oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

Any of the modified chemistries or formats of RNA oligonucleotides described herein can be combined with each other, and that one, two, three, four, five, or more different types of modifications can be included within the same molecule.

In some embodiments, the RNA oligonucleotide may comprise at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. Nos. 7,399,845, 7,741,457, 8,022,193, 7,569,686, 7,335,765, 7,314,923, 7,335,765, and 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

### Expression Constructs

Some aspects of this invention provide expression constructs that encode any of the fusion proteins described herein. For example the expression constructs may encode an RNA binding protein fused to an ARRDC1 protein (e.g., ARRDC1:Tat) or an RNA binding protein fused to one or more WW domains. In some embodiments, the expression constructs described herein may further encode, or encode separately, a binding RNA. It should be appreciated that the binding RNA may be expressed under the control of the same promoter sequence or a different promoter sequence as any of the fusion proteins described herein. In some embodiments, an expression construct encoding a binding RNA is co-expressed with any of the expression constructs described herein. In some embodiments, the expression constructs described herein may further encode, or encode separately, a cargo RNA. In some embodiments, the cargo RNA is expressed under the control of the same promoter sequence or a different promoter sequence as any of the fusion proteins or binding RNAs provided herein. In some embodiments, the cargo RNA is expressed as part of the same transcript as the binding RNA. For example, the binding RNA and the cargo RNA may be expressed as a single transcript. In some embodiments, the construct encodes a cargo RNA that is fused 5′ to the binding RNA. In some embodiments, the construct encodes a cargo RNA that is fused 3′ to the binding RNA. In some embodiments, the construct encodes a cargo RNA and a binding RNA that are fused via one or more linkers. It should be appreciated that the cargo RNA may also be expressed as a separate transcript from the binding RNA. When expressed as a separate transcript, the cargo RNA may comprise a sequence that binds to the binding RNA (e.g., via complementary base pairing). Accordingly, in some embodiments, the construct encodes a cargo RNA that may comprise a nucleotide sequence that is complementary to a sequence of a binding RNA. In some embodiments, the cargo RNA is expressed from a separate expression construct from the construct encoding the RNA binding protein and/or the binding RNA. In some embodiments, the cargo RNA is expressed from the same construct (e.g., expression vector) encoding the RNA binding protein and/or the binding RNA, but under a different promoter.

In some embodiments, the expression constructs described herein may further encode a gene product or gene products that induce or facilitate the generation of ARMMs in cells harboring such a construct. In some embodiments, the expression constructs encode an ARRDC1 protein, or variant thereof, and/or a TSG101 protein, or variant thereof. In some embodiments, overexpression of either or both of these gene products in a cell increase the production of ARMMs in the cell, thus turning the cell into a microvesicle producing cell. In some embodiments, such an expression construct comprises at least one restriction or recombination site that allows in-frame cloning of an RNA binding protein sequence to be fused, either at the C-terminus, or at the N-terminus of the encoded ARRDC1, or variant thereof. As another example an expression construct comprises at least one restriction or recombination site that allows in-frame cloning of an RNA binding protein sequence to be fused either at the C-terminus, or at the N-terminus of one ore more encoded WW domains.

In some embodiments, the expression construct comprises (a) a nucleotide sequence encoding an ARRDC1 protein, or variant thereof, operably linked to a heterologous promoter, and (b) a restriction site or a recombination site positioned adjacent to the ARRDC1-encoding nucleotide sequence allowing for the insertion of an RNA binding protein or RNA binding protein variant sequence in frame with the ARRDC1-encoding nucleotide sequence. In certain embodiments, the expression constructs encode a fusion protein comprising an ARRDC1 protein, or variant thereof, and a Tat protein or variant thereof.

Some aspects of this invention provide an expression construct comprising (a) a nucleotide sequence encoding a WW domain, or variant thereof, operably linked to a heterologous promoter, and (b) a restriction site or a recombination site positioned adjacent to the WW domain-encoding nucleotide sequence allowing for the insertion of an RNA binding protein or RNA binding protein variant sequence in frame with the WW domain-encoding nucleotide sequence. The expression constructs may encode an RNA binding protein fused to at least one WW domain. In some embodiments, the expression constructs encode an RNA binding protein, or variant thereof, fused to at least one WW domain, or variant thereof. Any of the expression constructs, described herein, may encode any WW domain or variant thereof. For example, the expression constructs may comprise any nucleotide sequence capable of encoding a WW domain or variant thereof from the poly peptide sequence (SEQ ID NO: 6); (SEQ ID NO: 7); (SEQ ID NO: 8); (SEQ ID NO: 9); (SEQ ID NO: 10); (SEQ ID NO: 11); (SEQ ID NO: 12); (SEQ ID NO: 13); (SEQ ID NO: 14); (SEQ ID NO: 18) or (SEQ ID NO: 19).

The expression constructs, described herein, may comprise any nucleic acid sequence capable of encoding a WW domain or variant thereof. For example a nucleic acid sequence encoding a WW domain or WW domain variant may be from the human ubiquitin ligase WWP1, WWP2, Nedd4-1, Nedd4-2, Smurf1, Smurf2, ITCH, NEDL1, or NEDL2. Exemplary nucleic acid sequences of WW domain containing proteins are listed below. It should be appreciated that any of the nucleic acids encoding WW domains or WW domain variants of the exemplary proteins may be used in the invention, described herein, and are not meant to be limiting.

Human WWP1 nucleic acid sequence (uniprot.org/uniprot/Q9H0M0).

(SEQ ID NO: 23)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC

Human WWP2 nucleic acid sequence (uniprot.org/uniprot/O00308).

(SEQ ID NO: 24)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC

Human Nedd4-1 nucleic acid sequence (uniprot.org/uniprot/P46934).

(SEQ ID NO: 25)
ACAGTTGCCTGCCCTGGGCGGGGGCGAGCGCGTCCGGTTTGCTGGAAGCGTTCGGAAATG
GCAACTTGCGCGGTGGAGGTGTTCGGGCTCCTGGAGGACGAGGAAAATTCACGAATTGTG
AGAGTAAGAGTTATAGCCGGAATAGGCCTTGCCAAGAAGGATATATTGGGAGCTAGTGAT
CCTTACGTGAGAGTGACGTTATATGACCCAATGAATGGAGTTCTTACAAGTGTGCAAACA
AAAACCATTAAAAAGAGTTTGAATCCAAAGTGGAATGAAGAAATATTATTCAGAGTTCAT
CCTCAGCAGCACCGGCTTCTTTTTGAAGTGTTTGACGAAAACCGATTGACAAGAGATGAT
TTCCTAGGTCAAGTGGATGTTCCACTTTATCCATTACCGACAGAAAATCCAAGATTGGAG
AGACCATATACATTTAAGGATTTTGTTCTTCATCCAAGAAGTCACAAATCAAGAGTTAAA
GGTTATCTGAGACTAAAAATGACTTATTTACCTAAAACCAGTGGCTCAGAAGATGATAAT
GCAGAACAGGCTGAGGAATTAGAGCCTGGCTGGGTTGTTTTGGACCAACCAGATGCTGCT
TGCCATTTGCAGCAACAACAAGAACCTTCTCCTCTACCTCCAGGGGGGAAGAGAGGCAG
GATATCCTTGGAAGGACCTATTATGTAAACCATGAATCTAGAAGAACACAGTGGAAAAGA
CCAACCCCTCAGGACAACCTAACAGATGCTGAGAATGGCAACATTCAACTGCAAGCACAA
CGTGCATTTACCACCAGGCGGCAGATATCCGAGGAAACAGAAAGTGTTGACAACCAAGAG
TCTTCCGAGAACTGGGAAATTATAAGAGAAGATGAAGCCACCATGTATAGCAGCCAGGCC
TTCCCATCACCTCCACCGTCAAGTAACTTGGATGTTCCAACTCATCTTGCAGAAGAATTG
AATGCCAGACTCACCATTTTTGGAAATTCAGCCGTGAGCCAGCCAGCATCGAGCTCAAAT
CATTCCAGCAGAAGAGGCAGCTTACAAGCCTATACTTTTGAGGAACAACCTACACTTCCT
GTGCTTTTGCCTACTTCATCTGGATTACCACCAGGTTGGGAAGAAAAACAAGATGAAAGA
GGAAGATCATATTATGTAGATCACAATTCCAGAACGACTACTTGGACAAAGCCCACTGTA
CAGGCCACAGTGGAGACCAGTCAGCTGACCTCAAGCCAGAGTTCTGCAGGCCCTCAATCA
CAAGCCTCCACCAGTGATTCAGGCCAGCAGGTGACCCAGCCATCTGAAATTGAGCAAGGA
TTCCTTCCTAAAGGCTGGGAAGTCCGGCATGCACCAAATGGGAGGCCTTTCTTTATTGAC
CACAACACTAAAACCACCACCTGGGAAGATCCAAGATTGAAAATTCCAGCCCATCTGAGA
GGAAAGACATCACTTGATACTTCCAATGATCTAGGGCCTTTACCTCCAGGATGGGAAGAG
AGAACTCACACAGATGGAAGAATCTTCTACATAAATCACAATATAAAAAGAACACAATGG
GAAGATCCTCGGTTGGAGAATGTAGCAATAACTGGACCAGCAGTGCCCTACTCCAGGGAT
TACAAAAGAAAGTATGAGTTCTTCCGAAGAAAGTTGAAGAAGCAGAATGACATTCCAAAC
AAATTTGAAATGAAACTTCGCCGAGCAACTGTTCTTGAAGACTCTTACCGGAGAATTATG
GGTGTCAAGAGAGCAGACTTCCTGAAGGCTCGACTGTGGATTGAGTTTGATGGTGAAAAG
GGATTGGATTATGGAGGAGTTGCCAGAGAATGGTTCTTCCTGATCTCAAAGGAAATGTTT
AACCCTTATTATGGGTTGTTTGAATATTCTGCTACGGACAATTATACCCTACAGATAAAT
CCAAACTCTGGATTGTGTAACGAAGATCACCTCTCTTACTTCAAGTTTATTGGTCGGGTA
GCTGGAATGGCAGTTTATCATGGCAAACTGTTGGATGGTTTTTTCATCCGCCCATTTTAC
AAGATGATGCTTCACAAACCAATAACCCTTCATGATATGGAATCTGTGGATAGTGAATAT
TACAATTCCCTAAGATGGATTCTTGAAAATGACCCAACAGAATTGGACCTCAGGTTTATC
ATAGATGAAGAACTTTTTGGACAGACACATCAACATGAGCTGAAAAATGGTGGATCAGAA
ATAGTTGTCACCAATAAGAACAAAAAGGAATATATTTATCTTGTAATACAATGGCGATTT
GTAAACCGAATCCAGAAGCAAATGGCTGCTTTTAAAGAGGGATTCTTTGAACTAATACCA
CAGGATCTCATCAAAATTTTTGATGAAAATGAACTAGAGCTTCTTATGTGTGGACCGGGA
GATGTTGATGTGAATGACTGGAGGGAACATACAAAGTATAAAAATGGCTACAGTGCAAAT
CATCAGGTTATACAGTGGTTTTGGAAGGCTGTTTTAATGATGGATTCAGAAAAAAGAATA
AGATTACTTCAGTTTGTCACTGGCACATCTCGGGTGCCTATGAATGGATTTGCTGAACTA
TACGGTTCAAATGGACCACAGTCATTTACAGTTGAACAGTGGGGTACTCCTGAAAAGCTG
CCAAGAGCTCATACCTGTTTTAATCGCCTGGACTTGCCACCTTATGAATCATTTGAAGAA
TTATGGGATAAACTTCAGATGGCAATTGAAAACACCCAGGGCTTTGATGGAGTTGATTAG
ATTACAAATAACAATCTGTAGTGTTTTTACTGCCATAGTTTTATAACCAAAATCTTGACT
TAAAATTTTCCGGGGAACTACTAAAATGTGGCCACTGAGTCTTCCCAGATCTTGAAGAAA
ATCATATAAAAAGCATTTGAAGAAATAGTACGAC

Human Nedd4-2 nucleic acid sequence (>gi|345478679|ref|NM_015277.5|Homo sapiens neural precursor cell expressed, developmentally down-regulated 4-like, E3 ubiquitin protein ligase (NEDD4L), transcript variant d, mRNA).

(SEQ ID NO: 26)
ATGGCGACCGGGCTCGGGGAGCCGGTCTATGGACTTTCCGAAGACGAGGGAGAGTCCCGTAT
TCTCAGAGTAAAAGTTGTTTCTGGAATTGATCTCGCCAAAAAGGACATCTTTGGAGCCAGTG
ATCCGTATGTGAAACTTTCATTGTACGTAGCGGATGAGAATAGAGAACTTGCTTTGGTCCAG
ACAAAAACAATTAAAAAGACACTGAACCCAAAATGGAATGAAGAATTTTATTTCAGGGTAAA
CCCATCTAATCACAGACTCCTATTTGAAGTATTTGACGAAAATAGACTGACACGAGACGACT
TCCTGGGCCAGGTGGACGTGCCCCTTAGTCACCTTCCGACAGAAGATCCAACCATGGAGCGA
CCCTATACATTTAAGGACTTTCTCCTCAGACCAAGAAGTCATAAGTCTCGAGTTAAGGGATT
TTTGCGATTGAAAATGGCCTATATGCCAAAAAATGGAGGTCAAGATGAAGAAAACAGTGACC
AGAGGGATGACATGGAGCATGGATGGGAAGTTGTTGACTCAAATGACTCGGCTTCTCAGCAC
CAAGAGGAACTTCCTCCTCCTCCTCTGCCTCCCGGGTGGGAAGAAAAAGTGGACAATTTAGG
CCGAACTTACTATGTCAACCACAACAACCGGACCACTCAGTGGCACAGACCAAGCCTGATGG
ACGTGTCCTCGGAGTCGGACAATAACATCAGACAGATCAACCAGGAGGCAGCACACCGGCGC
TTCCGCTCCCGCAGGCACATCAGCGAAGACTTGGAGCCCGAGCCCTCGGAGGGCGGGGATGT
CCCCGAGCCTTGGGAGACCATTTCAGAGGAAGTGAATATCGCTGGAGACTCTCTCGGTCTGG
CTCTGCCCCCACCACCGGCCTCCCCAGGATCTCGGACCAGCCCTCAGGAGCTGTCAGAGGAA
CTAAGCAGAAGGCTTCAGATCACTCCAGACTCCAATGGGGAACAGTTCAGCTCTTTGATTCA
AAGAGAACCCTCCTCAAGGTTGAGGTCATGCAGTGTCACCGACGCAGTTGCAGAACAGGGCC
ATCTACCACCGCCATCAGTGGCCTATGTACATACCACGCCGGGTCTGCCTTCAGGCTGGGAA
GAAAGAAAAGATGCTAAGGGGCGCACATACTATGTCAATCATAACAATCGAACCACAACTTG
GACTCGACCTATCATGCAGCTTGCAGAAGATGGTGCGTCCGGATCAGCCACAAACAGTAACA
ACCATCTAATCGAGCCTCAGATCCGCCGGCCTCGTAGCCTCAGCTCGCCAACAGTAACTTTA
TCTGCCCCGCTGGAGGGTGCCAAGGACTCACCCGTACGTCGGGCTGTGAAAGACACCCTTTC
CAACCCACAGTCCCCACAGCCATCACCTTACAACTCCCCCAAACCACAACACAAAGTCACAC
AGAGCTTCTTGCCACCCGGCTGGGAAATGAGGATAGCGCCAAACGGCCGGCCCTTCTTCATT
GATCATAACACAAAGACTACAACCTGGGAAGATCCACGTTTGAAATTTCCAGTACATATGCG
GTCAAAGACATCTTTAAACCCCAATGACCTTGGCCCCCTTCCTCCTGGCTGGGAAGAAAGAA
TTCACTTGGATGGCCGAACGTTTTATATTGATCATAATAGCAAAATTACTCAGTGGGAAGAC
CCAAGACTGCAGAACCCAGCTATTACTGGTCCGGCTGTCCCTTACTCCAGAGAATTTAAGCA
GAAATATGACTACTTCAGGAAGAAATTAAAGAAACCTGCTGATATCCCCAATAGGTTTGAAA
TGAAACTTCACAGAAATAACATATTTGAAGAGTCCTATCGGAGAATTATGTCCGTGAAAAGA
CCAGATGTCCTAAAAGCTAGACTGTGGATTGAGTTTGAATCAGAGAAAGGTCTTGACTATGG
GGGTGTGGCCAGAGAATGGTTCTTCTTACTGTCCAAAGAGATGTTCAACCCCTACTACGGCC
TCTTTGAGTACTCTGCCACGGACAACTACACCCTTCAGATCAACCCTAATTCAGGCCTCTGT
AATGAGGATCATTTGTCCTACTTCACTTTTATTGGAAGAGTTGCTGGTCTGGCCGTATTTCA
TGGGAAGCTCTTAGATGGTTTCTTCATTAGACCATTTTACAAGATGATGTTGGGAAAGCAGA
TAACCCTGAATGACATGGAATCTGTGGATAGTGAATATTACAACTCTTTGAAATGGATCCTG
GAGAATGACCCTACTGAGCTGGACCTCATGTTCTGCATAGACGAAGAAAACTTTGGACAGAC
ATATCAAGTGGATTTGAAGCCCAATGGGTCAGAAATAATGGTCACAAATGAAAACAAAAGGG
AATATATCGACTTAGTCATCCAGTGGAGATTTGTGAACAGGGTCCAGAAGCAGATGAACGCC
TTCTTGGAGGGATTCACAGAACTACTTCCTATTGATTTGATTAAAATTTTTGATGAAAATGA
GCTGGAGTTGCTCATGTGCGGCCTCGGTGATGTGGATGTGAATGACTGGAGACAGCATTCTA
TTTACAAGAACGGCTACTGCCCAAACCACCCCGTCATTCAGTGGTTCTGGAAGGCTGTGCTA
CTCATGGACGCCGAAAAGCGTATCCGGTTACTGCAGTTTGTCACAGGGACATCGCGAGTACC
TATGAATGGATTTGCCGAACTTTATGGTTCCAATGGTCCTCAGCTGTTTACAATAGAGCAAT
GGGGCAGTCCTGAGAAACTGCCCAGAGCTCACACATGCTTTAATCGCCTTGACTTACCTCCA
TATGAAACCTTTGAAGATTTACGAGAGAAACTTCTCATGGCCGTGGAAAATGCTCAAGGATT
TGAAGGGGTGGATTAA

Human Smurf1 nucleic acid sequence (uniprot.org/uniprot/Q9HCE7).

(SEQ ID NO: 27)
ATGTCGAACCCCGGGACACGCAGGAACGGCTCCAGCATCAAGATCCGTCTGACAGTGTTA
TGTGCCAAGAACCTTGCAAAGAAAGACTTCTTCAGGCTCCCTGACCCTTTTGCAAAGATT
GTCGTGGATGGGTCTGGGCAGTGCCACTCAACCGACACTGTGAAAAACACATTGGACCCA
AAGTGGAACCAGCACTATGATCTATATGTTGGGAAAACGGATTCGATAACCATTAGCGTG
TGGAACCATAAGAAAATTCACAAGAAACAGGGAGCTGGCTTCCTGGGCTGTGTGCGGCTG
CTCTCCAATGCCATCAGCAGATTAAAAGATACCGGATACCAGCGTTTGGATCTATGCAAA
CTAAACCCCTCAGATACTGATGCAGTTCGTGGCCAGATAGTGGTCAGTTTACAGACACGA
GACAGAATAGGAACCGGCGGCTCGGTGGTGGACTGCAGAGGACTGTTAGAAAATGAAGGA
ACGGTGTATGAAGACTCCGGGCCTGGGAGGCCGCTCAGCTGCTTCATGGAGGAACCAGCC
CCTTACACAGATAGCACCGGTGCTGCTGCTGGAGGAGGGAATTGCAGGTTCGTGGAGTCC
CCAAGTCAAGATCAAAGACTTCAGGCACAGCGGCTTCGAAACCCTGATGTGCGAGGTTCA
CTACAGACGCCCCAGAACCGACCACACGGCCACCAGTCCCCGGAACTGCCCGAAGGCTAC
GAACAAAGAACAACAGTCCAGGGCCAAGTTTACTTTTTGCATACACAGACTGGAGTTAGC
ACGTGGCACGACCCCAGGATACCAAGTCCCTCGGGGACCATTCCTGGGGGAGATGCAGCT
TTTCTATACGAATTCCTTCTACAAGGCCATACATCTGAGCCCAGAGACCTTAACAGTGTG
AACTGTGATGAACTTGGACCACTGCCGCCAGGCTGGGAAGTCAGAAGTACAGTTTCTGGG
AGGATATATTTTGTAGATCATAATAACCGAACAACCCAGTTTACAGACCCAAGGTTACAC
CACATCATGAATCACCAGTGCCAACTCAAGGAGCCCAGCCAGCCGCTGCCACTGCCCAGT
GAGGGCTCTCTGGAGGACGAGGAGCTTCCTGCCCAGAGATACGAAAGAGATCTAGTCCAG
AAGCTGAAAGTCCTCAGACACGAACTGTCGCTTCAGCAGCCCCAAGCTGGTCATTGCCGC
ATCGAAGTGTCCAGAGAAGAAATCTTTGAGGAGTCTTACCGCCAGATAATGAAGATGCGA
CCGAAAGACTTGAAAAAACGGCTGATGGTGAAATTCCGTGGGGAAGAAGGTTTGGATTAC
GGTGGTGTGGCCAGGGAGTGGCTTTACTTGCTGTGCCATGAAATGCTGAATCCTTATTAC
GGGCTCTTCCAGTATTCTACGGACAATATTTACATGTTGCAAATAAATCCGGATTCTTCA
ATCAACCCCGACCACTTGTCTTATTTCCACTTTGTGGGGCGGATCATGGGGCTGGCTGTG
TTCCATGGACACTACATCAACGGGGGCTTCACAGTGCCCTTCTACAAGCAGCTGCTGGGG
AAGCCCATCCAGCTCTCAGATCTGGAATCTGTGGACCCAGAGCTGCATAAGAGCTTGGTG
TGGATCCTAGAGAACGACATCACGCCTGTACTGGACCACACCTTCTGCGTGGAACACAAC
GCCTTCGGGCGGATCCTGCAGCATGAACTGAAACCCAATGGCAGAAATGTGCCAGTCACA
GAGGAGAATAAGAAAGAATACGTCCGGTTGTATGTAAACTGGAGGTTTATGAGAGGAATC
GAAGCCCAGTTCTTAGCTCTGCAGAAGGGGTTCAATGAGCTCATCCCTCAACATCTGCTG
AAGCCTTTTGACCAGAAGGAACTGGAGCTGATCATAGGCGGCCTGGATAAAATAGACTTG
AACGACTGGAAGTCGAACACGCGGCTGAAGCACTGTGTGGCCGACAGCAACATCGTGCGG
TGGTTCTGGCAAGCGGTGGAGACGTTCGATGAAGAAAGGAGGGCCAGGCTCCTGCAGTTT
GTGACTGGGTCCACGCGAGTCCCGCTCCAAGGCTTCAAGGCTTTGCAAGGTTCTACAGGC
GCGGCAGGGCCCCGGCTGTTCACCATCCACCTGATAGACGCGAACACAGACAACCTTCCG
AAGGCCCATACCTGCTTTAACCGGATCGACATTCCACCATATGAGTCCTATGAGAAGCTC
TACGAGAAGCTGCTGACAGCCGTGGAGGAGACCTGCGGGTTTGCTGTGGAGTGA

Human Smurf2 nucleic acid sequence (uniprot.org/uniprot/Q9HAU4).

(SEQ ID NO: 28)
ATGTCTAACCCCGGACGCCGGAGGAACGGGCCCGTCAAGCTGCGCCTGACAGTACTCTGT
GCAAAAAACCTGGTGAAAAAGGATTTTTTCCGACTTCCTGATCCATTTGCTAAGGTGGTG
GTTGATGGATCTGGGCAATGCCATTCTACAGATACTGTGAAGAATACGCTTGATCCAAAG
TGGAATCAGCATTATGACCTGTATATTGGAAAGTCTGATTCAGTTACGATCAGTGTATGG
AATCACAAGAAGATCCATAAGAAACAAGGTGCTGGATTTCTCGGTTGTGTTCGTCTTCTT
TCCAATGCCATCAACCGCCTCAAAGACACTGGTTATCAGAGGTTGGATTTATGCAAACTC
GGGCCAAATGACAATGATACAGTTAGAGGACAGATAGTAGTAAGTCTTCAGTCCAGAGAC
CGAATAGGCACAGGAGGACAAGTIGTGGACTGCAGTCGTTTATTTGATAACGATTTACCA
GACGGCTGGGAAGAAAGGAGAACCGCCTCTGGAAGAATCCAGTATCTAAACCATATAACA
AGAACTACGCAATGGGAGCGCCCAACACGACCGGCATCCGAATATTCTAGCCCTGGCAGA
CCTCTTAGCTGCTTTGTTGATGAGAACACTCCAATTAGTGGAACAAATGGTGCAACATGT
GGACAGTCTTCAGATCCCAGGCTGGCAGAGAGGAGAGTCAGGTCACAACGACATAGAAAT
TACATGAGCAGAACACATTTACATACTCCTCCAGACCTACCAGAAGGCTATGAACAGAGG
ACAACGCAACAAGGCCAGGTGTATTTCTTACATACACAGACTGGTGTGAGCACATGGCAT
GATCCAAGAGTGCCCAGGGATCTTAGCAACATCAATTGTGAAGAGCTTGGTCCATTGCCT
CCTGGATGGGAGATCCGTAATACGGCAACAGGCAGAGTTTATTTCGTTGACCATAACAAC
AGAACAACACAATTTACAGATCCTCGGCTGTCTGCTAACTTGCATTTAGTTTTAAATCGG
CAGAACCAATTGAAAGACCAACAGCAACAGCAAGTGGTATCGTTATGTCCTGATGACACA
GAATGCCTGACAGTCCCAAGGTACAAGCGAGACCTGGTTCAGAAACTAAAAATTTTGCGG
CAAGAACTTTCCCAACAACAGCCTCAGGCAGGTCATTGCCGCATTGAGGTTTCCAGGGAA
GAGATTTTTGAGGAATCATATCGACAGGTCATGAAAATGAGACCAAAAGATCTCTGGAAG
CGATTAATGATAAAATTTCGTGGAGAAGAAGGCCTTGACTATGGAGGCGTTGCCAGGGAA
TGGTTGTATCTCTTGTCACATGAAATGTTGAATCCATACTATGGCCTCTTCCAGTATTCA
AGAGATGATATTTATACATTGCAGATCAATCCTGATTCTGCAGTTAATCCGGAACATTTA
TCCTATTTCCACTTTGTTGGACGAATAATGGGAATGGCTGTGTTTCATGGACATTATATT
GATGGTGGTTTCACATTGCCTTTTTATAAGCAATTGCTTGGGAAGTCAATTACCTTGGAT
GACATGGAGTTAGTAGATCCGGATCTTCACAACAGTTTAGTGTGGATACTTGAGAATGAT
ATTACAGGTGTTTTGGACCATACCTTCTGTGTTGAACATAATGCATATGGTGAAATTATT
CAGCATGAACTTAAACCAAATGGCAAAAGTATCCCTGTTAATGAAGAAAATAAAAAAGAA
TATGTCAGGCTCTATGTGAACTGGAGATTTTTACGAGGCATTGAGGCTCAATTCTTGGCT
CTGCAGAAAGGATTTAATGAAGTAATTCCACAACATCTGCTGAAGACATTTGATGAGAAG
GAGTTAGAGCTCATTATTTGTGGACTTGGAAAGATAGATGTTAATGACTGGAAGGTAAAC
ACCCGGTTAAAACACTGTACACCAGACAGCAACATTGTCAAATGGTTCTGGAAAGCTGTG
GAGTTTTTTGATGAAGAGCGACGAGCAAGATTGCTTCAGTTTGTGACAGGATCCTCTCGA
GTGCCTCTGCAGGGCTTCAAAGCATTGCAAGGTGCTGCAGGCCCGAGACTCTTTACCATA
CACCAGATTGATGCCTGCACTAACAACCTGCCGAAAGCCCACACTTGCTTCAATCGAATA
GACATTCCACCCTATGAAAGCTATGAAAAGCTATATGAAAAGCTGCTAACAGCCATTGAA
GAAACATGTGGATTTGCTGTGGAATGA

Human ITCH nucleic acid sequence (uniprot.org/uniprot/Q96J02).

(SEQ ID NO: 29)
GGAGTCGCCGCCGCCCCGAGTTCCGGTACCATGCATTTCACGGTGGCCTTGTGGAGACAA
CGCCTTAACCCAAGGAAGTGACTCAAACTGTGAGAACTCCAGGTTTTCCAACCTATTGGT
GGTATGTCTGACAGTGGATCACAACTTGGTTCAATGGGTAGCCTCACCATGAAATCACAG
CTTCAGATCACTGTCATCTCAGCAAAACTTAAGGAAAATAAGAAGAATTGGTTTGGACCA
AGTCCTTACGTAGAGGTCACAGTAGATGGACAGTCAAAGAAGACAGAAAAATGCAACAAC
ACAAACAGTCCCAAGTGGAAGCAACCCCTTACAGTTATCGTTACCCCTGTGAGTAAATTA
CATTTTCGTGTGTGGAGTCACCAGACACTGAAATCTGATGTTTTGTTGGGAACTGCTGCA
TTAGATATTTATGAAACATTAAAGTCAAACAATATGAAACTTGAAGAAGTAGTTGTGACT
TTGCAGCTTGGAGGTGACAAAGAGCCAACAGAGACAATAGGAGACTTGTCAATTTGTCTT
GATGGGCTACAGTTAGAGTCTGAAGTTGTTACCAATGGTGAAACTACATGTTCAGAAAGT
GCTTCTCAGAATGATGATGGCTCCAGATCCAAGGATGAAACAAGAGTGAGCACAAATGGA
TCAGATGACCCTGAAGATGCAGGAGCTGGTGAAAATAGGAGAGTCAGTGGGAATAATTCT
CCATCACTCTCAAATGGTGGTTTTAAACCTTCTAGACCTCCAAGACCTTCACGACCACCA
CCACCCACCCCACGTAGACCAGCATCTGTCAATGGTTCACCATCTGCCACTTCTGAAAGT
GATGGGTCTAGTACAGGCTCTCTGCCGCCGACAAATACAAATACAAATACATCTGAAGGA
GCAACATCTGGATTAATAATTCCTCTTACTATATCTGGAGGCTCAGGCCCTAGGCCATTA
AATCCTGTAACTCAAGCTCCCTTGCCACCTGGTTGGGAGCAGAGAGTGGACCAGCACGGG
CGAGTTTACTATGTAGATCATGTTGAGAAAAGAACAACATGGGATAGACCAGAACCTCTA
CCTCCTGGCTGGGAACGGCGGGTTGACAACATGGGACGTATTTATTATGTTGACCATTTC
ACAAGAACAACAACGTGGCAGAGGCCAACACTGGAATCCGTCCGGAACTATGAACAATGG
CAGCTACAGCGTAGTCAGCTTCAAGGAGCAATGCAGCAGTTTAACCAGAGATTCATTTAT
GGGAATCAAGATTTATTTGCTACATCACAAAGTAAAGAATTTGATCCTCTTGGTCCATTG
CCACCTGGATGGGAGAAGAGAACAGACAGCAATGGCAGAGTATATTTCGTCAACCACAAC
ACACGAATTACACAATGGGAAGACCCCAGAAGTCAAGGTCAATTAAATGAAAAGCCCTTA
CCTGAAGGTTGGGAAATGAGATTCACAGTGGATGGAATTCCATATTTTGTGGACCACAAT
AGAAGAACTACCACCTATATAGATCCCCGCACAGGAAAATCTGCCCTAGACAATGGACCT
CAGATAGCCTATGTTCGGGACTTCAAAGCAAAGGTTCAGTATTTCCGGTTCTGGTGTCAG
CAACTGGCCATGCCACAGCACATAAAGATTACAGTGACAAGAAAAACATTGTTTGAGGAT
TCCTTTCAACAGATAATGAGCTTCAGTCCCCAAGATCTGCGAAGACGTTTGTGGGTGATT
TTTCCAGGAGAAGAAGGTTTAGATTATGGAGGTGTAGCAAGAGAATGGTTCTTTCTTTTG
TCACATGAAGTGTTGAACCCAATGTATTGCCTGTTTGAATATGCAGGGAAGGATAACTAC
TGCTTGCAGATAAACCCCGCTTCTTACATCAATCCAGATCACCTGAAATATTTTCGTTTT
ATTGGCAGATTTATTGCCATGGCTCTGTTCCATGGGAAATTCATAGACACGGGTTTTTCT
TTACCATTCTATAAGCGTATCTTGAACAAACCAGTTGGACTCAAGGATTTAGAATCTATT
GATCCAGAATTTTACAATTCTCTCATCTGGGTTAAGGAAAACAATATTGAGGAATGTGAT
TTGGAAATGTACTTCTCCGTTGACAAAGAAATTCTAGGTGAAATTAAGAGTCATGATCTG
AAACCTAATGGTGGCAATATTCTTGTAACAGAAGAAAATAAAGAGGAATACATCAGAATG
GTAGCTGAGTGGAGGTTGTCTCGAGGTGTTGAAGAACAGACACAAGCTTTCTTTGAAGGC
TTTAATGAAATTCTTCCCCAGCAATATTTGCAATACTTTGATGCAAAGGAATTAGAGGTC
CTTTTATGTGGAATGCAAGAGATTGATTTGAATGACTGGCAAAGACATGCCATCTACCGT
CATTATGCAAGGACCAGCAAACAAATCATGTGGTTTTGGCAGTTTGTTAAAGAAATTGAT
AATGAGAAGAGAATGAGACTTCTGCAGTTTGTTACTGGAACCTGCCGATTGCCAGTAGGA
GGATTTGCTGATCTCATGGGGAGCAATGGACCACAGAAATTCTGCATTGAAAAAGTTGGG
AAAGAAAATTGGCTACCCAGAAGTCATACCTGTTTTAATCGCCTGGACCTGCCACCATAC
AAGAGCTATGAGCAACTGAAGGAAAAGCTGTTGTTTGCCATAGAAGAAACAGAAGGATTT
GGACAAGAGTAACTTCTGAGAACTTGCACCATGAATGGGCAAGAACTTATTTGCAATGTT
TGTCCTTCTCTGCCTGTTGCACATCTTGTAAAATTGGACAATGGCTCTTTAGAGAGTTAT
CTGAGTGTAAGTAAATTAATGTTCTCATTTAAAAAAAAAAAAAAAAAAA

Human NEDL1 nucleic acid sequence (uniprot.org/uniprot/Q76N89).

(SEQ ID NO: 30)
GCGCATCAGGCGCTGTTGTTGGAGCCGGAACACCGTGCGACTCTGACCGAACCGGCCCCC
TCCTCGCGCACACACTCGCCGAGCCGCGCGCGCCCCTCCGCCGTGACAGTGGCCGTGGCC
TCCGCTCTCTCGGGGCACCCGGCAGCCAGAGCGCAGCGAGAGCGGGCGGTCGCCAGGGTC
CCCTCCCCAGCCAGTCCCAGGCGCCCGGTGCACTATGCGGGGCACGTGCGCCCCCCAGCT
CTAATCTGCGCGCTGACAGGAGCATGATCTGTGCCCAGGCCAGGGCTGCCAAGGAATTGA
TGCGCGTACACGTGGTGGGTCATTATGCTGCTACACCTGTGTAGTGTGAAGAATCTGTAC
CAGAACAGGTTTTTAGGCCTGGCCGCCATGGCGTCTCCTTCTAGAAACTCCCAGAGCCGA
CGCCGGTGCAAGGAGCCGCTCCGATACAGCTACAACCCCGACCAGTTCCACAACATGGAC
CTCAGGGGCGGCCCCCACGATGGCGTCACCATTCCCCGCTCCACCAGCGACACTGACCTG
GTCACCTCGGACAGCCGCTCCACGCTCATGGTCAGCAGCTCCTACTATTCCATCGGGCAC
TCTCAGGACCTGGTCATCCACTGGGACATAAAGGAGGAAGTGGACGCTGGGGACTGGATT
GGCATGTACCTCATTGATGAGGTCTTGTCCGAAAACTTTCTGGACTATAAAAACCGTGGA
GICAATGGTTCTCATCGGGGCCAGATCATCTGGAAGATCGATGCCAGCTCGTACTTTGTG
GAACCTGAAACTAAGATCTGCTTCAAATACTACCATGGAGTGAGTGGGGCCCTGCGAGCA
ACCACCCCCAGTGTCACGGTCAAAAACTCGGCAGCTCCTATTTTTAAAAGCATTGGTGCT
GATGAGACCGTCCAAGGACAAGGAAGTCGGAGGCTGATCAGCTTCTCTCTCTCAGATTTC
CAAGCCATGGGGTTGAAGAAAGGGATGTTTTTCAACCCAGACCCTTATCTGAAGATTTCC
ATTCAGCCTGGGAAACACAGCATCTTCCCCGCCCTCCCTCACCATGGACAGGAGAGGAGA
TCCAAGATCATAGGCAACACCGTGAACCCCATCTGGCAGGCCGAGCAATTCAGTTTTGTG
TCCTTGCCCACTGACGTGCTGGAAATTGAGGTGAAGGACAAGTTTGCCAAGAGCCGCCCC
ATCATCAAGCGCTTCTTGGGAAAGCTGTCGATGCCCGTTCAAAGACTCCTGGAGAGACAC
GCCATAGGGGATAGGGTGGTCAGCTACACACTTGGCCGCAGGCTTCCAACAGATCATGTG
AGTGGACAGCTGCAATTCCGATTTGAGATCACTTCCTCCATCCACCCAGATGATGAGGAG
ATTTCCCTGAGTACCGAGCCTGAGTCAGCCCAAATTCAGGACAGCCCCATGAACAACCTG
ATGGAAAGCGGCAGTGGGGAACCTCGGTCTGAGGCACCAGAGTCCTCTGAGAGCTGGAAG
CCAGAGCAGCTGGGTGAGGGCAGTGTCCCCGATGGTCCAGGGAACCAAAGCATAGAGCTT
TCCAGACCAGCTGAGGAAGCAGCAGTCATCACGGAGGCAGGAGACCAGGGCATGGTCTCT
GTGGGACCTGAAGGGGCTGGGGAGCTCCTGGCCCAGGTGCAAAAGGACATCCAGCCTGCC
CCCAGTGCAGAAGAGCTGGCCGAGCAGCTGGACCTGGGTGAGGAGGCATCAGCACTGCTG
CTGGAAGACGGTGAAGCCCCAGCCAGCACCAAGGAGGAGCCCTTGGAGGAGGAAGCAACG
ACCCAGAGCCGGGCTGGAAGGGAAGAAGAGGAGAAGGAGCAGGAGGAGGAGGGAGATGTG
TCTACCCTGGAGCAGGGAGAGGGCAGGCTGCAGCTGCGGGCCTCGGTGAAGAGAAAAAGC
AGGCCCTGCTCCTTGCCTGTGTCCGAGCTGGAGACGGTGATCGCGTCAGCCTGCGGGGAC
CCCGAGACCCCGCGGACACACTACATCCGCATCCACACCCTGCTGCACAGCATGCCCTCC
GCCCAGGGGGGCAGCGCGGCAGAGGAGGAGGACGGCGCGGAGGAGGAGTCCACCCTCAAG
GACTCCTCGGAGAAGGATGGGCTCAGCGAGGIGGACACGGTGGCCGCTGACCCGTCTGCC
CTGGAAGAGGACAGAGAAGAGCCCGAGGGGGCTACTCCAGGCACGGCGCACCCTGGCCAC
TCCGGGGGCCACTTCCCCAGCCTGGCCAATGGCGCGGCCCAGGATGGCGACACGCACCCC
AGCACCGGGAGCGAGAGCGACTCCAGCCCCAGGCAAGGCGGGGACCACAGTTGCGAGGGC
TGTGACGCGTCCTGCTGCAGCCCCTCGTGCTACAGCTCCTCGTGCTACAGCACGTCCTGC
TACAGCAGCTCGTGCTACAGCGCCTCGTGCTACAGCCCCTCCTGCTACAACGGCAACAGG
TTCGCCAGCCACACGCGCTTCTCCTCCGTGGACAGCGCCAAGATCTCCGAGAGCACGGTC
TTCTCCTCGCAAGACGACGAGGAGGAGGAGAACAGCGCGTTCGAGTCGGTACCCGACTCC
ATGCAGAGCCCTGAGCTGGACCCGGAGTCCACGAACGGCGCTGGGCCGTGGCAAGACGAG
CTGGCCGCCCCTAGCGGGCACGTGGAAAGAAGCCCGGAAGGTCTGGAATCCCCCGTGGCA
GGTCCAAGCAATCGGAGAGAAGACTGGGAAGCTCGAATTGACAGCCACGGGCGGGTCTTT
TATGTGGACCACGTGAACCGCACAACCACCTGGCAGCGTCCGACGGCAGCAGCCACCCCG
GATGGCATGCGGAGATCGGGGTCCATCCAGCAGATGGAGCAACTCAACAGGCGGTATCAA
AACATTCAGCGAACCATTGCAACAGAGAGGTCCGAAGAAGATTCTGGCAGCCAAAGCTGC
GAGCAAGCCCCAGCAGGAGGAGGCGGAGGTGGAGGGAGTGACTCAGAAGCCGAATCTTCC
CAGTCCAGCTTAGATCTAAGGAGAGAGGGGTCACTTTCTCCAGTGAACTCACAAAAAATC
ACCTTGCTGCTGCAGTCCCCAGCGGTCAAGTTCATCACCAACCCCGAGTTCTTCACTGTG
CTACACGCCAATTATAGTGCCTACCGAGTCTTCACCAGTAGCACCTGCTTAAAGCACATG
ATTCTGAAAGTCCGACGGGATGCTCGCAATTTTGAACGCTACCAGCACAACCGGGACTTG
GTGAATTTCATCAACATGTTCGCAGACACTCGGCTGGAACTGCCCCGGGGCTGGGAGATC
AAAACGGACCAGCAGGGAAAGTCTTTTTTCGTGGACCACAACAGTCGAGCTACCACTTTC
ATTGACCCCCGAATCCCTCTTCAGAACGGTCGTCTTCCCAATCATCTAACTCACCGACAG
CACCTCCAGAGGCTCCGAAGTTACAGCGCCGGAGAGGCCTCAGAAGTTTCTAGAAACAGA
GGAGCCTCTTTACTGGCCAGGCCAGGACACAGCTTAGTAGCTGCTATTCGAAGCCAACAT
CAACATGAGTCATTGCCACTGGCATATAATGACAAGATTGTGGCATTTCTTCGCCAGCCA
AACATTTTTGAAATGCTGCAAGAGCGTCAGCCAAGCTTAGCAAGAAACCACACACTCAGG
GAGAAAATCCATTACATTCGGACTGAGGGTAATCACGGGCTTGAGAAGTIGTCCTGTGAT
GCGGATCTGGTCATTTTGCTGAGTCTCTTTGAAGAAGAGATTATGTCCTACGTCCCCCTG
CAGGCTGCCTTCCACCCTGGGTATAGCTTCTCTCCCCGATGTTCACCCTGTTCTTCACCT
CAGAACTCCCCAGGITTACAGAGAGCCAGIGCAAGAGCCCCTTCCCCCTACCGAAGAGAC
TTTGAGGCCAAGCTCCGCAATTTCTACAGAAAACTGGAAGCCAAAGGATTTGGTCAGGGT
CCGGGGAAAATTAAGCTCATTATTCGCCGGGATCATTTGTTGGAGGGAACCTTCAATCAG
GTGATGGCCTATTCGCGGAAAGAGCTCCAGCGAAACAAGCTCTACGTCACCTTTGTTGGA
GAGGAGGGCCTGGACTACAGTGGCCCCTCGCGGGAGTTCTTCTTCCTTCTGTCTCAGGAG
CTCTTCAACCCTTACTATGGACTCTTTGAGTACTCGGCAAATGATACTTACACGGTGCAG
ATCAGCCCCATGTCCGCATTTGTAGAAAACCATCTTGAGTGGTTCAGGTTTAGCGGTCGC
ATCCTGGGTCTGGCTCTGATCCATCAGTACCTTCTTGACGCTTTCTTCACGAGGCCCTTC
TACAAGGCACTCCTGAGACTGCCCTGTGATTTGAGTGACCTGGAATATTTGGATGAGGAA
TTCCACCAGAGTTTGCAGTGGATGAAGGACAACAACATCACAGACATCTTAGACCTCACT
TTCACTGTTAATGAAGAGGTTTTTGGACAGGTCACGGAAAGGGAGTTGAAGTCTGGAGGA
GCCAACACACAGGTGACGGAGAAAAACAAGAAGGAGTACATCGAGCGCATGGTGAAGTGG
CGGGTGGAGCGCGGCGTGGTACAGCAGACCGAGGCGCTGGTGCGCGGCTICTACGAGGTT
GTAGACTCGAGGCTGGTGTCCGTGTTTGATGCCAGGGAGCTGGAGCTGGIGATAGCTGGC
ACCGCGGAAATCGACCTAAATGACTGGCGGAATAACACTGAGTACCGGGGAGGTTACCAC
GATGGGCATCTTGTGATCCGCTGGTTCTGGGCTGCGGTGGAGCGCTTCAATAATGAGCAG
AGGCTGAGATTACTGCAGTTTGTCACGGGAACATCCAGCGTGCCCTACGAAGGCTTCGCA
GCCCTCCGTGGGAGCAATGGGCTTCGGCGCTTCTGCATAGAGAAATGGGGGAAAATTACT
TCTCTCCCCAGGGCACACACATGCTTCAACCGACTGGATCTTCCACCGTATCCCTCGTAC
TCCATGTTGTATGAAAAGCTGTTAACAGCAGTAGAGGAAACCAGCACCTTTGGACTTGAG
TGAGGACATGGAACCTCGCCTGACATTTTCCTGGCCAGTGACATCACCCTTCCTGGGATG
ATCCCCTTTTCCCTTTCCCTTAATCAACTCTCCTTTGATTTTGGTATTCCATGATTTTTA
TTTTCAAAC

Human NEDL2 nucleic acid sequence (uniprot.org/uniprot/Q9P2P5).

(SEQ ID NO: 31)
AGAGTTCCATCAGAGCCTGCAGTGGATGAAAGACAATGATATCCATGACATCCTAGACCT
CACGTTCACTGTGAACGAAGAAGTATTTGGGCAGATAACTGAACGAGAATTAAAGCCAGG
GGGTGCCAATATCCCAGTTACAGAGAAGAACAAGAAGGAGTACATCGAGAGGATGGTGAA
GTGGAGGATTGAGAGGGGTGTTGTACAGCAAACAGAGAGCTTAGTGCGTGGCTTCTATGA
GGTGGTGGATGCCAGGCTGGTATCTGTTTTTGATGCAAGAGAACTGGAATTGGTCATCGC
AGGCACAGCTGAAATAGACCTAAGTGATTGGAGAAACAACACAGAATATAGAGGAGGATA
CCATGACAATCATATTGTAATTCGGTGGTTCTGGGCTGCAGTGGAAAGATTCAACAATGA
ACAACGACTAAGGTTGTTACAGTTTGTTACAGGCACATCCAGCATTCCCTATGAAGGATT
TGCTTCACTCCGAGGGAGTAACGGCCCAAGAAGATTCTGTGTGGAGAAATGGGGGAAAAT
CACTGCTCTTCCCAGAGCGCATACATGTTTTAACCGTCTGGATCTGCCTCCCTACCCATC
CTTTTCCATGCTTTATGAAAAACTGTTGACAGCAGTTGAAGAAACCAGTACTTTTGGACT
TGAGTGACCTGGAAGCTGAATGCCCATCTCTGTGGACAGGCAGTTTCAGAAGCTGCCTTC
TAGAAGAATGATTGAACATTGGAAGTTTCAAGAGGATGCTTCCTTTAGGATAAAGCTACG
TGCTGTTGTTTTCCAGGAACAAGTGCTCTGTCACATTTGGGGACTGGAGATGAGTCCTCT
TGGAAGGATTTGGGTGAGCTTGATGCCCAGGGAACAACCCAACCGTCTTTCAATCAACAG
TTCTTGACTGCCAAACTTTTTCCATTTGTTATGTTCCAAGACAAAGATGAACCCATACAT
GATCAGCTCCACGGTAATTTTTAGGGACTCAGGAGAATCTTGAAACTTACCCTTGAACGT
GGTTCAAGCCAAACTGGCAGCATTTGGCCCAATCTCCAAATTAGAGCAAGTTAAATAATA
TAATAAAAGTAAATATATTTCCTGAAAGTACATTCATTTAAGCCCTAAGTTATAACAGAA
TATTCATTTCTTGCTTATGAGTGCCTGCATGGTGTGCACCATAGGTTTCCGCTTTCATGG
GACATGAGTGAAAATGAAACCAAGTCAATATGAGGTACCTTTACAGATTTGCAATAAGAT
GGTCTGTGACAATGTATATGCAAGTGGTATGTGTGTAATTATGGCTAAAGACAAACCATT
ATTCAGTGAATTACTAATGACAGATTTTATGCTTTATAATGCATGAAAACAATTTTAAAA
TAACTAGCAATTAATCACAGCATATCAGGAAAAAGTACACAGTGAGTTCTGTTTATTTTT
TGTAGGCTCATTATGTTTATGTTCTTTAAGATGTATATAAGAACCTACTTATCATGCTGT
ATGTATCACTCATTCCATTTTCATGTTCCATGCATACTCGGGCATCATGCTAATATGTAT
CCTTTTAAGCACTCTCAAGGAAACAAAAGGGCCTTTTATTTTTATAAAGGTAAAAAAAAT
TCCCCAAATATTTTGCACTGAATGTACCAAAGGTGAAGGGACATTACAATATGACTAACA
GCAACTCCATCACTTGAGAAGTATAATAGAAAATAGCTTCTAAATCAAACTTCCTTCACA
GTGCCGTGTCTACCACTACAAGGACTGTGCATCTAAGTAATAATTTTTTAAGATTCACTA
TATGTGATAGTATGATATGCATTTATTTAAAATGCATTAGACTCTCTTCCATCCATCAAA
TACTTTACAGGATGGCATTTAATACAGATATTTCGTATTTCCCCCACTGCTTTTTATTTG
TACAGCATCATTAAACACTAAGCTCAGTTAAGGAGCCATCAGCAACACTGAAGAGATCAG
TAGTAAGAATTCCATTTTCCCTCATCAGTGAAGACACCACAAATTGAAACTCAGAACTAT
ATTTCTAAGCCTGCATTTTCACTGATGCATAATTTTCTTATTAATATTAAGAGACAGTTT
TTCTATGGCATCTCCAAAACTGCATGACATCACTAGTCTTACTTCTGCTTAATTTTATGA
GAAGGTATTCTTCATTTTAATTGCTTTTGGGATTACTCCACATCTTTGTTTATTTCTTGA
CTAATCAGATTTTCAATAGAGTGAAGTTAAATTGGGGGTCATAAAAGCATTGGATTGACA
TATGGTTTGCCAGCCTATGGGTTTACAGGCATTGCCCAAACATTTCTTTGAGATCTATAT
TTATAAGCAGCCATGGAATTCCTATTATGGGATGTTGGCAATCTTACATTTTATAGAGGT
CATATGCATAGTTTTCATAGGTGTTTTGTAAGAACTGATTGCTCTCCTGTGAGTTAAGCT
ATGTTTACTACTGGGACCCTCAAGAGGAATACCACTTATGTTACACTCCTGCACTAAAGG
CACGTACTGCAGTGTGAAGAAATGTTCTGAAAAAGGGTTATAGAAATCTGGAAATAAGAA
AGGAAGAGCTCTCTGTATTCTATAATTGGAAGAGAAAAAAAGAAAAACTTTTAACTGGAA
ATGTTAGTTTGTACTTATTGATCATGAATACAAGTATATATTTAATTTTGCAAAAAAAAA
AAAAAAAAAAAAAAG

In certain embodiments, the nucleic acids may encode RNA binding proteins having two WW domains or WW domain variants from the human ITCH protein having the nucleic acid sequence: CCCTTGCCACCTGGTTGGGAGCAGAGAGTGGACCAGCACGGGCGAGTTTACTAT GTAGATCATGTTGAGAAAAGAACAACATGGGATAGACCAGAACCTCTACCTCCT GGCTGGGAACGGCGGGTTGACAACATGGGACGTATTTATTATGTTGACCATTTCA CAAGAACAACAACGTGGCAGAGGCCAACACTG (SEQ ID NO: 32). In other embodiments, the nucleic acids may encode RNA binding proteins having four WW domains or WW domain variants from the human ITCH protein having the nucleic acid sequence: CCCTTGCCACCTGGTTGGGAGCAGAGAGTGGACCAGCACGGGCGAGTTTACTAT GTAGATCATGTTGAGAAAAGAACAACATGGGATAGACCAGAACCTCTACCTCCT GGCTGGGAACGGCGGGTTGACAACATGGGACGTATTATTATGTTGACCATTTCA CAAGAACAACAACGTGGCAGAGGCCAACACTGGAATCCGTCCGGAACTATGAAC AATGGCAGCTACAGCGTAGTCAGCTTCAAGGAGCAATGCAGCAGTTTAACCAGA GATTCATTTATGGGAATCAAGATTTATTTGCTACATCACAAAGTAAAGAATTTGA TCCTCTTGGTCCATTGCCACCTGGATGGGAGAAGAGAACAGACAGCAATGGCAG AGTATATTTCGTCAACCACAACACACGAATTACACAATGGGAAGACCCCAGAAG TCAAGGTCAATTAAATGAAAAGCCCTTACCTGAAGGTTGGGAAATGAGATTCAC AGTGGATGGAATTCCATATTTTGTGGACCACAATAGAAGAACTACCACCTATATA GATCCCCGCACA (SEQ ID NO: 33). The nucleic acid constructs that encode the RNA binding proteins, described herein, that are fused to at least one WW domain or WW domain variant are non-naturally occurring, that is, they do not exist in nature.

In some embodiments the expression constructs comprise a nucleic acid sequence encoding a WW domain, or variant thereof from the nucleic acid sequence (SEQ ID NO: 23); (SEQ ID NO: 24); (SEQ ID NO: 25); (SEQ ID NO: 26); (SEQ ID NO: 27); (SEQ ID NO: 28); (SEQ ID NO: 29); (SEQ ID NO: 30); (SEQ ID NO: 31); (SEQ ID NO: 32) or (SEQ ID NO: 33). In certain embodiments, the expression constructs encode a fusion protein comprising a WW domain or multiple WW domains, and a Tat protein or variant thereof.

Some aspects of this invention provide expression constructs that encode any of the binding RNAs, cargo RNAs, or fusions of any of the binding RNAs and cargo RNAs described herein. In some embodiments, the expression construct comprises (a) a nucleotide sequence encoding a binding RNA, or variant thereof, operably linked to a heterologous promoter, and (b) a restriction site or a recombination site positioned adjacent to the binding RNA-encoding nucleotide sequence allowing for the insertion of a cargoRNA-encoding nucleotide sequence. In some embodiments, the expression construct comprises (a) a nucleotide sequence encoding a cargo RNA, or variant thereof, operably linked to a heterologous promoter, and (b) a restriction site or a recombination site positioned adjacent to the cargo RNA-encoding nucleotide sequence allowing for the insertion of a binding RNA-encoding nucleotide sequence. In certain embodiments, the expression constructs encode a TAR binding RNA, or variant thereof fused to a cargo RNA. In some embodiments, the cargo RNA is an mRNA.

Nucleic acids encoding any of the fusion proteins, binding RNAs, and/or cargoRNAs, described herein, may be in any number of nucleic acid “vectors” known in the art. As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Non-viral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for production of a virus capable of infecting, entering, or being introduced to a cell to deliver nucleic acid therein.

Expression of any of the fusion proteins, binding RNAs, and/or cargoRNAs, described herein, may be controlled by any regulatory sequence (e.g. a promoter sequence) known in the art. Regulatory sequences, as described herein, are nucleic acid sequences that regulate the expression of a nucleic acid sequence. A regulatory or control sequence may include sequences that are responsible for expressing a particular nucleic acid (e.g., a ARRDC1:Tat fusion protein) or may include other sequences, such as heterologous, synthetic, or partially synthetic sequences. The sequences can be of eukaryotic, prokaryotic or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell, and introns. A heterologous regulatory region is not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

The term operably linked refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken β-actin (CBA) promoter, and a hybrid form of the CBA promoter (CBh).

### Cells Producing Microvesicles Containing RNA Binding Proteins, Binding RNAs, and Cargo RNAs

A microvesicle-producing cell of the present invention may be a cell containing any of the expression constructs, any of the fusion proteins, any of the binding RNAs, any of the cargo RNAs, and/or any of the binding RNAs fused to any of the cargo RNAs described herein. For example, an inventive microvesicle-producing cell may contain one or more recombinant expression constructs encoding (1) an ARRDC1 protein, or PSAP (SEQ ID NO: 1) motif-containing variant thereof and (2) an RNA binding protein (e.g., a Tat protein), that is associated with the ARRDC1 protein, or PSAP (SEQ ID NO: 1) motif-containing variant thereof. In some embodiments, a microvesicle-producing cell may contain one or more recombinant expression constructs encoding (1) an ARRDC1 protein, or PSAP (SEQ ID NO: 1) motif-containing variant thereof, and (2) an RNA binding protein fused to at least one WW domain, or variant thereof, under the control of a heterologous promoter. In certain embodiments, an expression construct in the microvesicle producing cell encodes a binding RNA that associates (e.g., binds specifically) with the RNA binding protein. In some embodiments, an expression construct in the microvesicle producing cell encodes a cargo RNA that associates with the binding RNA. For example, the construct may encode a binding RNA that is fused to a cargo RNA. In some embodiments, the microvesicle-producing cell may express a binding RNA and a cargo RNA from different expression constructs or express a binding RNA and a cargo RNA under the control of different promoters.

Any of the expression constructs, described herein, may be stably inserted into the genome of the cell. In some embodiments, the expression construct is maintained in the cell, but not inserted into the genome of the cell. In some embodiments, the expression construct is in a vector, for example, a plasmid vector, a cosmid vector, a viral vector, or an artificial chromosome. In some embodiments, the expression construct further comprises additional sequences or elements that facilitate the maintenance and/or the replication of the expression construct in the microvesicle-producing cell, or that improve the expression of the fusion protein in the cell. Such additional sequences or elements may include, for example, an origin of replication, an antibiotic resistance cassette, a polyA sequence, and/or a transcriptional isolator. Some expression constructs suitable for the generation of microvesicle producing cells according to aspects of this invention are described elsewhere herein. Methods and reagents for the generation of additional expression constructs suitable for the generation of microvesicle producing cells according to aspects of this invention will be apparent to those of skill in the art based on the present disclosure. In some embodiments, the microvesicle producing cell is a mammalian cell, for example, a mouse cell, a rat cell, a hamster cell, a rodent cell, or a nonhuman primate cell. In some embodiments, the microvesicle producing cell is a human cell.

One skilled in the art may employ conventional techniques, such as molecular or cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, New York, and Sambrook et al. Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S.J. Higgins eds. (1985)); Transcription And Translation Hames & Higgins, eds. (1984); Animal Cell Culture (R I. Freshney, ed. (1986)); Immobilized Cells And Enzymes (IRL Press, (1986)); Gennaro et al. (eds.) Remington's Pharmaceutical Sciences, 18th edition; B. Perbal, A Practical Guide To Molecular Cloning (1984); F. M. Ausubel et al. (eds.), Current Protocols in Molecular Biology, John Wiley & Sons, Inc. (updates through 2001), Coligan et al. (eds.), Current Protocols in Immunology, John Wiley & Sons, Inc. (updates through 2001); W. Paul et al. (eds.) Fundamental Immunology, Raven Press; E. J. Murray et al. (ed.) Methods in Molecular Biology: Gene Transfer and Expression Protocols, The Humana Press Inc. (1991)(especially vol. 7); and J. E. Celis et al., Cell Biology: A Laboratory Handbook, Academic Press (1994).

### Delivery of ARMMs Containing RNA Binding Proteins, Binding RNAs and Cargo RNAs.

The inventive microvesicles (e.g., ARMMs) containing any of the expression constructs, any of the fusion proteins, any of the binding RNAs, any of the cargo RNAs, and/or any of the binding RNAs fused to any of the cargo RNAs, described herein, may further have a targeting moiety. The targeting moiety may be used to target the delivery of ARMMs to specific cell types, resulting in the release of the contents of the ARMM into the cytoplasm of the specific targeted cell type. A targeting moiety may selectively bind an antigen of the target cell. For example, the targeting moiety may be a membrane-bound immunoglobulin, an integrin, a receptor, a receptor ligand, an aptamer, a small molecule, or a variant thereof. Any number of cell surface proteins may also be included in an ARMM to facilitate the binding of an ARMM to a target cell and/or to facilitate the uptake of an ARMM into a target cell. Integrins, receptor tyrosine kinases, G-protein coupled receptors, and membrane-bound immunoglobulins suitable for use with embodiments of this invention will be apparent to those of skill in the art and the invention is not limited in this respect. For example, in some embodiments, the integrin is an α1β1, α2β1, α4β1, α5β1, α6β1, αLβ2, αMβ2, αIIbβ3, αVβ3, αVβ5, αVβ6, or αα6β4 integrin. In some embodiments, the receptor tyrosine kinase is a an EGF receptor (ErbB family), insulin receptor, PDGF receptor, FGF receptor, VEGF receptor, HGF receptor, Trk receptor, Eph receptor, AXL receptor, LTK receptor, TIE receptor, ROR receptor, DDR receptor, RET receptor, KLG receptor, RYK receptor, or MuSK receptor. In some embodiments, the G-protein coupled receptor is a rhodopsin-like receptor, the secretin receptor, metabotropic glutamate/pheromone receptor, cyclic AMP receptor, frizzled/smoothened receptor, CXCR4, CCR5, or beta-adrenergic receptor.

Any number of membrane-bound immunoglobulins, known in the art, may be used as targeting moieties to target the delivery of ARMMs containing a cargo protein to any number of target cell types. In certain embodiments, the membrane-bound immunoglobulin targeting moiety binds a tumor associated or tumor specific antigen. Some non-limiting examples of tumor antigens include, CA19-9, c-met, PD-1, CTLA-4, ALK, AFP, EGFR, Estrogen receptor (ER), Progesterone receptor (PR), HER2/neu, KIT, B-RAF, S100, MAGE, Thyroglobulin, MUC-1, and PSMA (Bigbee W., et al. “Tumor markers and immunodiagnosis.”, Cancer Medicine. 6th ed. Hamilton, Ontario, Canada: BC Decker Inc., 2003.; Andriole G, et al. “Mortality results from a randomized prostate-cancer screening trial.”, New England Journal of Medicine, 360(13):1310-1319, 2009.; Schröder F H, et al. “Screening and prostate-cancer mortality in a randomized European study.” New England Journal of Medicine, 360(13):1320-1328, 2009.; Buys S S, et al. “Effect of screening on ovarian cancer mortality: the Prostate, Lung, Colorectal and Ovarian (PLCO) Cancer Screening Randomized Controlled Trial.”, JAMA, 305(22):2295-2303, 2011.; Cramer D W et al. “Ovarian cancer biomarker performance in prostate, lung, colorectal, and ovarian cancer screening trial specimens.” Cancer Prevention Research, 4(3):365-374, 2011.; Roy D M, et al. “Candidate prognostic markers in breast cancer: focus on extracellular proteases and their inhibitors.”, Breast Cancer. July 3; 6:81-91, 2014.; Tykodi S S. et al. “PD-1 as an emerging therapeutic target in renal cell carcinoma: current evidence.” Onco Targets Ther. July 25; 7:1349-59, 2014.; and Weinberg R A. The Biology of Cancer, Garland Science, Taylor & Francis Group LLC, New York, NY, 2007.; the entire contents of each are incorporated herein by reference).

In certain embodiments, the membrane-bound immunoglobulin targeting moiety binds to an antigen of a specific cell type. The cell type may be a stem cell, such as a pluripotent stem cell. Some non-limiting examples of antigens specific to pluripotent stem cells include Oct4 and Nanog, which were the first proteins identified as essential for both early embryo development and pluripotency maintenance in embryonic stem cells (Nichols J, et al. “Formation of pluripotent stem cells in the mammalian embryo depends on the POU transcription factor Oct4.”, Cell. 95:379-91, 1998; the contents of which are hereby incorporated by reference). In addition to Oct4, Sox2 and Nanog, many other pluripotent stem cell markers have been identified, including Sal14, Dax1, Essrb, Tbx3, Tcl1, Rif1, Nac1 and Zfp281 (Loh Y, et al. “The Oct4 and Nanog transcription network regulates pluripotency in mouse embryonic stem cells.”, Nat Genet. 38:431-40, 2006). The membrane-bound immunoglobulin targeting moiety may also bind to an antigen of a differentiated cell type. For example, the targeting moiety may bind to an antigen specific for a lung epithelial cell to direct the delivery of an ARMM cargo RNA to lung epithelial cells. As a non-limiting example, a membrane-bound immunoglobulin targeting moiety may bind to the alveolar epithelial type 1 cell specific protein RTI₄₀ or HTI₅₆ to deliver cargo proteins to alveolar epithelial type 1 cells (McElroy M C et al. “The use of alveolar epithelial type I cell-selective markers to investigate lung injury and repair.”, European Respiratory Jorunal 24:4, 664-673, 2004; the entire contents of which are hereby incorporated by reference). As another example, the targeting moiety may bind a mucin, such as muc5ac, or muc5b. It should be appreciated that the examples of antigens provided in this application are not limiting and the targeting moiety may be any moiety capable of binding any cellular antigen known in the art.

Some aspects of this invention relate to the recognition that ARMMs are taken up by target cells, and ARMM uptake results in the release of the contents of the ARMM into the cytoplasm of the target cells. In some embodiments, the cargo RNA is an agent that affects a desired change in the target cell, for example, a change in cell survival, proliferation rate, a change in differentiation stage, a change in a cell identity, a change in chromatin state, a change in the transcription rate of one or more genes, a change in the transcriptional profile, or a post-transcriptional change in gene compression of the target cell. It will be understood by those of skill in the art, that the agent to be delivered (e.g., cargo RNA) will be chosen according to the desired effect in the target cell.

Using any of the cargo RNAs, described herein, or any of the therapeutic RNAs known in the art, expression of one or more genes in a target cell may be modulated

In some embodiments, cells from a subject are obtained and a cargo RNA is delivered to the cells by a system or method provided herein ex vivo. In some embodiments, the treated cells are selected for those cells in which a desired gene is expressed or repressed. In some embodiments, treated cells carrying a desired cargo RNA are returned to the subject they were obtained from.

As another example, to augment the differentiation stage of a target cell, for example, to reprogram a differentiated target cell into an embryonic stem cell-like stage, the cell is contacted, in some embodiments, with ARMMs with cargo RNAs that express reprogramming factors, for example, mRNAs that express Oct4, Sox2, c-Myc, and/or KLF4. Similarly, to affect the change in the chromatin state of a target cell, the cell is contacted, in some embodiments, with ARMMs containing a cargo RNA that expresses a chromatin modulator, for example, a DNA methyltransferase, or a histone deacetylase. As another example, if survival of the target cell is to be diminished, the target cell, in some embodiments, is contacted with ARMMs comprising a cytotoxic agent, for example, an mRNA that expresses a cytotoxic protein, or an siRNA that inhibits expression of a protein in a target cell that promotes survival. Additional cargo RNAs suitable for inclusion into ARMMs and for a ARMM-mediated delivery to a target cell or target cell population will be apparent to those skilled in the art, and the invention is not limited in this respect.

In some embodiments, the ARMMs comprising any of the fusion proteins, any of the binding RNAs, any of the cargo RNAs, and/or any of the binding RNAs fused to any of the cargo RNAs, described herein, further include a detectable label. Such ARMMs allow for the labeling of a target cell without genetic manipulation. Detectable labels suitable for direct delivery to target cells are known in the art, and include, but are not limited to, fluorescent proteins, fluorescent dyes, membrane-bound dyes, and enzymes, for example, membrane-bound or cytosolic enzymes, catalyzing the reaction resulting in a detectable reaction product. Detectable labels suitable according to some aspects of this invention further include membrane-bound antigens, for example, membrane-bound ligands that can be detected with commonly available antibodies or antigen binding agents.

In some embodiments, ARMMs are provided that comprise a cargo RNA that encodes a transcription factor, a transcriptional repressor, a fluorescent protein, a kinase, a phosphatase, a protease, a ligase, a chromatin modulator, or a recombinase. In some embodiments, ARMMs are provided that comprise a cargo RNA (e.g., an siRNA) that inhibits expression of a transcription factor, a transcriptional repressor, a fluorescent protein, a kinase, a phosphatase, a protease, a ligase, a chromatin modulator, or a recombinase. In some embodiments, the cargo RNA is a therapeutic RNA. In some embodiments the cargo RNA is an RNA that affects a change in the state or identity of a target cell. For example, in some embodiments, the cargo RNA encodes a reprogramming factor. Suitable transcription factors, transcriptional repressors, fluorescent proteins, kinases, phosphatases, proteases, ligases, chromatin modulators, recombinases, and reprogramming factors may be encoded by a cargo RNA that is associated with a binding RNA to facilitate their incorporation into ARMMs and their function may be tested by any methods that are known to those skilled in the art, and the invention is not limited in this respect.

Methods for isolating the ARMMs described herein are also provided. One exemplary method includes collecting the culture medium, or supernatant, of a cell culture comprising microvesicle-producing cells. In some embodiments, the cell culture comprises cells obtained from a subject, for example, cells suspected to exhibit a pathological phenotype, for example, a hyperproliferative phenotype. In some embodiments, the cell culture comprises genetically engineered cells producing ARMMs, for example, cells expressing a recombinant ARMM protein, for example, a recombinant ARRDC1 or TSG101 protein, such as an ARRDC1 or TSG101 protein fused to an RNA binding protein (e.g., a Tat protein) or variant thereof. In some embodiments, the supernatant is pre-cleared of cellular debris by centrifugation, for example, by two consecutive centrifugations of increasing G value (e.g., 500 G and 2000 G). In some embodiments, the method comprises passing the supernatant through a 0.2 μm filter, eliminating all large pieces of cell debris and whole cells. In some embodiments, the supernatant is subjected to ultracentrifugation, for example, at 120,000 G for 2 hours, depending on the volume of centrifugate. The pellet obtained comprises microvesicles. In some embodiments, exosomes are depleted from the microvesicle pellet by staining and/or sorting (e.g., by FACS or MACS) using an exosome marker as described herein. Isolated or enriched ARMMs can be suspended in culture media or a suitable buffer, as described herein.

### Methods of Microvesicle-Mediated Delivery of Cargo RNAs

Some aspects of this invention provide a method of delivering an agent, for example, a cargo RNA associated with a binding RNA (e.g., a P53-expressing RNA associated with a TAR element) to a target cell. In some embodiments, the cargo RNA is loaded into an ARMM by co-expressing in a cell, the cargo RNA associated with a binding RNA (e.g., a TAR element) and an ARRDC1 protein fused to an RNA binding protein (e.g., a Tat protein), or an RNA binding protein (e.g., a Tat protein) fused to a WW domain. The target cell can be contacted with an ARMM in different ways. For example, a target cell may be contacted directly with an ARMM as described herein, or with an isolated ARMM from a microvesicle producing cell. The contacting can be done in vitro by administering the ARMM to the target cell in a culture dish, or in vivo by administering the ARMM to a subject (e.g., parenterally or non-parenterally). In some embodiments, an ARMM is produced from a cell obtained from a subject. In some embodiments, the ARMM that was produced from a cell that was obtained from the subject is administered to the subject from which the ARMM producing cell was obtained. In some embodiments, the ARMM that was produced from a cell that was obtained from the subject is administered to a subject different from the subject from which the ARMM producing cell was obtained. As one example, a cell may be obtained from a subject and engineered to express one or more of the constructs provided herein (e.g., engineered to express a cargo RNA associated with a binding RNA, an ARRDC1 protein, an ARRDC1 protein fused to an RNA binding protein, and/or an RNA binding protein fused to a WW domain). The cell obtained from the subject and engineered to express one or more of the constructs provided herein may be administered to the same subject, or a different subject, from which the cell was obtained. Alternatively, the cell obtained from the subject and engineered to express one or more of the constructs provided herein produces ARMMs, which may be isolated and administered to the same subject form which the cell was obtained or administered to a different subject from which the cell was obtained.

Alternatively, a target cell can be contacted with a microvesicle producing cell as described herein, for example, in vitro by co-culturing the target cell and the microvesicle producing cell, or in vivo by administering a microvesicle producing cell to a subject harboring the target cell. Accordingly, the method may include contacting the target cell with a microvesicle, for example, an ARMM containing any of the cargo RNAs to be delivered, as described herein. The target cell may be contacted with a microvesicle-producing cell, as described herein, or with an isolated microvesicle that has a lipid bilayer, an ARRDC1 protein or variant thereof, a cargo RNA associated with a binding RNA and an RNA binding protein (e.g., a Tat protein) associated with ARRDC1 or a WW domain.

It should be appreciated that the target cell may be of any origin, for example from an organism. In some embodiments, the target cell is a mammalian cell. Some non-limiting examples of a mammalian cell include, without limitation, a mouse cell, a rat cell, hamster cell, a rodent cell, and a nonhuman primate cell. In some embodiments, the target cell is a human cell. It should also be appreciated that the target cell may be of any cell type. For example, the target cell may be a stem cell, which may include embryonic stem cells, induced pluripotent stem cells (iPS cells), fetal stem cells, cord blood stem cells, or adult stem cells (i.e., tissue specific stem cells). In other cases, the target cell may be any differentiated cell type found in a subject. In some embodiments, the target cell is a cell in vitro, and the method includes administering the microvesicle to the cell in vitro, or co-culturing the target cell with the microvesicle-producing cell in vitro. In some embodiments, the target cell is a cell in a subject, and the method comprises administering the microvesicle or the microvesicle-producing cell to the subject. In some embodiments, the subject is a mammalian subject, for example, a rodent, a mouse, a rat, a hamster, or a non-human primate. In some embodiments, the subject is a human subject.

In some embodiments, the target cell is a pathological cell. In some embodiments, the target cell is a cancer cell. In some embodiments, the microvesicle is associated with a binding agent that selectively binds an antigen on the surface of the target cell. In some embodiments, the antigen of the target cell is a cell surface antigen. In some embodiments, the binding agent is a membrane-bound immunoglobulin, an integrin, a receptor, or a receptor ligand. Suitable surface antigens of target cells, for example of specific target cell types, e.g. cancer cells, are known to those of skill in the art, as are suitable binding agents that specifically bind such antigens. Methods for producing membrane-bound binding agents, for example, membrane-bound immunoglobulins, membrane-bound antibodies or antibody fragments that specifically bind a surface antigen expressed on the surface of cancer cells, are also known to those of skill in the art. The choice of the binding agent will depend, of course, on the identity or the type of target cell. Cell surface antigens specifically expressed on various types of cells that can be targeted by ARMMs comprising membrane-bound binding agents will be apparent to those of skill in the art. It will be appreciated that the present invention is not limited in this respect.

### Co-Culture Systems

Some aspects of this invention provide in vitro cell culture systems having at least two types of cells: microvesicle producing cells, and target cells that take up the microvesicles produced. Accordingly, in the co-culture systems provided herein, there is a shuffling of the contents of the microvesicles (e.g., ARMMs) to the target cells. Such co-culture systems allow for the expression of a gene product or multiple gene products generated by the microvesicle producing cells in the target cells without genetic manipulation of the target cells.

In some embodiments, a co-culture system is provided that comprises (a) a microvesicle-producing cell population having a recombinant expression construct encoding (i) an ARRDC1 protein, or variant thereof fused to an RNA binding protein (e.g., Tat), under the control of a heterologous promoter, and/or (ii) an RNA binding protein (e.g., Tat) fused to a WW domain, under the control of a heterologous promoter, and/or (iii) an ARRDC1 protein, or variant thereof, under the control of a heterologous promoter, and/or (iv) a binding RNA (e.g., a TAR element) fused to a cargo RNA under the control of a heterologous promoter, and/or (v) a binding RNA (e.g., a TAR element) that associates with a cargo RNA, where the binding RNA and the cargo RNA are under the control of a heterologous promoter, and (b) a target cell population. In some embodiments, the ARRDC1 variant comprises a PSAP (SEQ ID NO: 1) motif. In other embodiments, the microvesicle comprises a TSG101 protein or variant thereof. In some embodiments, the TSG101 protein comprises a UEV domain.

In some embodiments, the microvesicle-producing cell comprises a plurality of expression constructs encoding a plurality of the proteins, fusion proteins and or RNAs provided herein. In some embodiments, the microvesicle-producing cell comprises the following recombinant expression constructs as described in the preceeding paragraph:

In some embodiments, the microvesicle-producing cell comprises one or more expression constructs encoding (i) an ARRDC1 protein, or variant thereof fused to an RNA binding protein (e.g., Tat), under the control of a heterologous promoter, and (iv) a binding RNA (e.g., a TAR element) fused to a cargo RNA under the control of a heterologous promoter.

In some embodiments, the microvesicle-producing cell comprises one or more expression constructs encoding (i) an ARRDC1 protein, or variant thereof fused to an RNA binding protein (e.g., Tat), under the control of a heterologous promoter, and (iv) a binding RNA (e.g., a TAR element) fused to a cargo RNA under the control of a heterologous promoter, and (iii) an ARRDC1 protein, or variant thereof, under the control of a heterologous promoter.

In some embodiments, the microvesicle-producing cell comprises one or more expression constructs encoding (i) an ARRDC1 protein, or variant thereof fused to an RNA binding protein (e.g., Tat), under the control of a heterologous promoter, and (v) a binding RNA (e.g., a TAR element) that associates with a cargo RNA, where the binding RNA and the cargo RNA are under the control of a heterologous promoter.

In some embodiments, the microvesicle-producing cell comprises one or more expression constructs encoding (i) an ARRDC1 protein, or variant thereof fused to an RNA binding protein (e.g., Tat), under the control of a heterologous promoter, and (v) a binding RNA (e.g., a TAR element) that associates with a cargo RNA, where the binding RNA and the cargo RNA are under the control of a heterologous promoter, and (iii) an ARRDC1 protein, or variant thereof, under the control of a heterologous promoter

In some embodiments, the microvesicle-producing cell comprises one or more expression constructs encoding (ii) an RNA binding protein (e.g., Tat) fused to a WW domain, under the control of a heterologous promoter, and (iv) a binding RNA (e.g., a TAR element) fused to a cargo RNA under the control of a heterologous promoter.

In some embodiments, the microvesicle-producing cell comprises one or more expression constructs encoding (ii) an RNA binding protein (e.g., Tat) fused to a WW domain, under the control of a heterologous promoter, and (iv) a binding RNA (e.g., a TAR element) fused to a cargo RNA under the control of a heterologous promoter, and (iii) an ARRDC1 protein, or variant thereof, under the control of a heterologous promoter.

In some embodiments, the microvesicle-producing cell comprises one or more expression constructs encoding (ii) an RNA binding protein (e.g., Tat) fused to a WW domain, under the control of a heterologous promoter, and (v) a binding RNA (e.g., a TAR element) that associates with a cargo RNA, where the binding RNA and the cargo RNA are under the control of a heterologous promoter.

In some embodiments, the microvesicle-producing cell comprises one or more expression constructs encoding (ii) an RNA binding protein (e.g., Tat) fused to a WW domain, under the control of a heterologous promoter, and (v) a binding RNA (e.g., a TAR element) that associates with a cargo RNA, where the binding RNA and the cargo RNA are under the control of a heterologous promoter, and (iii) an ARRDC1 protein, or variant thereof, under the control of a heterologous promoter.

One exemplary application of a co-culture system as provided herein is the programming or reprogramming of a target cell without genetic manipulation. For example, in some embodiments, the target cell is a differentiated cell, for example, a fibroblast cell. In some embodiments, the microvesicle producing cells are feeder cells or non-proliferating cells. In some embodiments, the microvesicle-producing cells produce ARMMs comprising one or more cargo RNAs that encode one or more reprogramming factors, (e.g., Oct4, Sox2, Klf4, and c-myc) that are fused to or are associated with a binding RNA. In other embodiments, the microvesicle-producing cells produce ARMMs comprising one or more cargo RNAs that interfere with the expression of one or more genes, for example a gene involved or associated with cell differentiation. In some embodiments, co-culture of the differentiated target cells with the microvesicle producing cells results in the reprogramming of the differentiated target cells to an embryonic state. In some embodiments, co-culture of the differentiated target cells with the microvesicle producing cells results in the programming, or trans-differentiation, of the target cells to a differentiated cell states that is different from the original cell state of the target cells.

Another exemplary application of a co-culture system, as provided herein, is the directed differentiation of embryonic stem cells. In some embodiments, the target cells are undifferentiated embryonic stem cells, and the microvesicle producing cells produce ARMMs comprising one or more cargo RNAs that encode one or more differentiation factors that are fused to or are associated with a binding RNA. Exemplary differentiation factors may include, but are not limited to signaling molecules or transcription factors that trigger or facilitate the differentiation of the embryonic stem cells into differentiated cells of a desired lineage, for example neuronal cells, or mesenchymal cells. In other embodiments, the microvesicle-producing cells produce ARMMs comprising one or more cargo RNAs that interfere with the expression of one or more genes, for example a gene involved or associated with undifferentiated cells.

Yet another exemplary application of a co-culture system, as provided herein, is the maintenance of stem cells, for example, of embryonic stem cells or of adult stem cells in an undifferentiated state. In some such embodiments, the microvesicle producing cells produce ARMMs comprising one or more cargo RNAs that encode one or more signaling molecules and/or transcription factors that are fused to or are associated with a binding RNA. In some embodiments, the one or more signaling molecules and/or transcription factors promote stem cell maintenance and/or inhibit stem cell differentiation. The microvesicle producing cells may create a microenvironment for the stem cells that mimics a naturally occurring stem cell niche. In other embodiments, the microvesicle-producing cells produce ARMMs comprising one or more cargo RNAs that interfere with the expression of one or more genes, for example by inhibiting expression of a gene involved or associated with inhibiting stem cell maintenance or promoting stem cell differentiation.

The microvesicle-producing cell of a culture system may be a cell of any type or origin that is capable of producing any of the ARMMs described herein. For example, the microvesicle-producing cell may be a mammalian cell, examples of which include but are not limited to, a cell from a rodent, a mouse, a rat, a hamster, or a non-human primate. The microvesicle-producing cell may also be from a human. One non-limiting example of a microvesicle-producing cell capable of producing an ARMM is a human embryonic kidney 293T cell. The microvesicle-producing cell may be a proliferating or a non-proliferating cell. In some embodiments, the microvesicle-producing cell is a feeder cell which supports the growth of other cells in the culture. Feeder cells may provide attachment substrates, nutrients, or other factors that are needed for the growth of cells in culture.

The target cell of the culture system can be a cell of any type or origin, which may be contacted with an ARMM from any of the microvesicle-producing cells, described herein. For example, the target cell may be a mammalian cell, examples of which include but are not limited to, a cell from a rodent, a mouse, a rat, a hamster, or a non-human primate. The target cell may also be from a human. The target cell may be from an established cell line (e.g., a 293T cell), or a primary cell cultured ex vivo (e.g., cells obtained from a subject and grown in culture). Target cells may be hematologic cells (e.g., hematopoietic stem cells, leukocytes, thrombocytes or erythrocytes), or cells from solid tissues, such as liver cells, kidney cells, lung cells, heart cells bone cells, skin cells, brain cells, or any other cell found in a subject. Cells obtained from a subject can be contacted with an ARMM from a microvesicle-producing cell and subsequently re-introduced into the same or another subject. In some embodiments, the target cell is a stem cell. The stem cell may be a totipotent stem cell that can differentiate into embryonic and extraembryonic cell types. The stem cell may also be a pluripotent stem cell, a multipotent stem cell, an oligopotent stem cell or a unipotent stem cell. In other embodiments, the target cell is a differentiated cell.

### Pharmaceutical Compositions

Other aspects of the present disclosure relate to pharmaceutical compositions comprising any of the ARMMs or microvesicle (e.g., ARMM) producing cells provided herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (24) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for delivering a cargo RNA (e.g. a cargo RNA that expresses a tumor suppressor) to a cell. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing an ARMM or microvesicle producing cell of the invention and (b) a second container containing a pharmaceutically acceptable diluent (e.g., sterile water) for injection. The pharmaceutically acceptable diluent can be used e.g., for reconstitution or dilution of the ARMM or microvesicle producing cell of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits, Vectors, Cells

Some aspects of this disclosure provide kits comprising a nucleic acid construct comprising a nucleotide sequence encoding one or more of any of the proteins (e.g., ARRDC1, and TSG101), fusion proteins (e.g., ARRDC1-Tat, and WW-Tat), and/or RNAs (e.g., TAR, TAR-cargoRNA ) provided herein. In some embodiments, the nucleotide sequence encodes any of the proteins, fusion proteins, and/or RNAs provided herein. In some embodiments, the nucleotide sequence comprises a heterologous promoter that drives expression of any of the proteins, fusion proteins, and/or RNAs provided herein.

Some aspects of this disclosure provide kits comprising a nucleic acid construct, comprising (a) a nucleotide sequence encoding an ARRDC1 protein fused to an RNA binding protein (e.g., Tat), or a fusion protein comprising a WW domain fused to an RNA binding protein (e.g., Tat) as provided herein, optionally wherein the nucleotide sequence encodes ARRDC1 and/or TSG101; and (b) a heterologous promoter that drives expression of the sequence of (a). In some embodiments, the kit further comprises an expression construct encoding a binding RNA (e.g., TAR) and/or a cargo RNA. In some embodiments, a further encodes a binding RNA (e.g., TAR) and/or a cargo RNA.

Some aspects of this disclosure provide microveslicle (e.g., ARMM) producing cells comprising any of the proteins, fusion proteins, and/or RNAs provided herein. In some embodiments, the cells comprise a nucleotide that encodes any of the proteins, fusion proteins, and/or RNAs provided herein. In some embodiments, the cells comprise any of the nucleotides or vectors provided herein.

The description of exemplary embodiments of the reporter systems above is provided for illustration purposes only and not meant to be limiting. Additional reporter systems, e.g., variations of the exemplary systems described in detail above, are also embraced by this disclosure.

It should be appreciated however, that additional proteins, fusion proteins, and RNAs would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1: Packaging Cargo RNAs into ARMMs Via Binding RNAs that Specifically Bind to RNA Binding Proteins

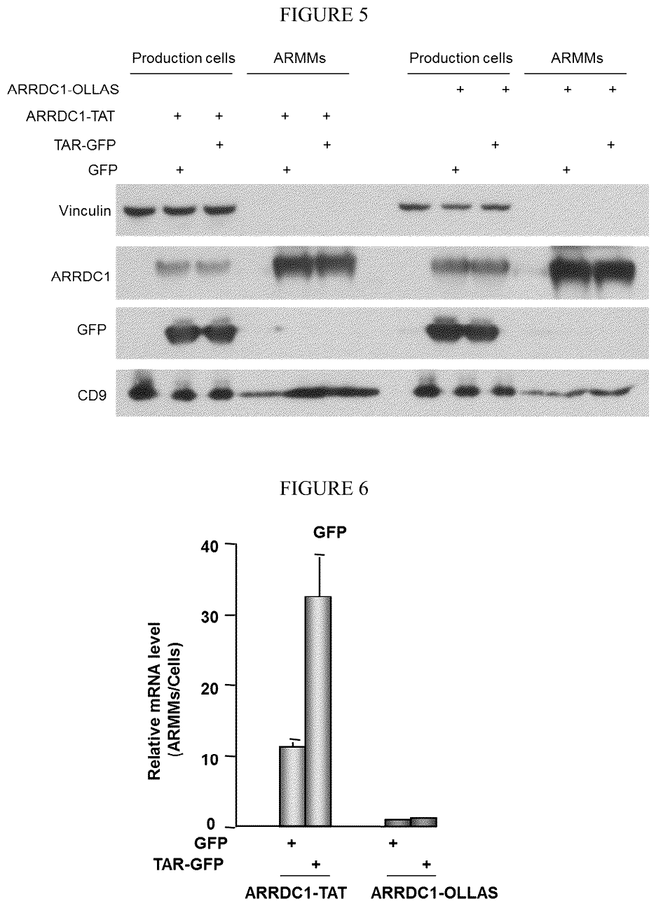

An ARRDC1 protein fused to Tat maintained the ability to bud out of cells as ARRDC1-containing ARMMs. For example, cells expressing either the ARRDC1-Tat fusion protein or the ARRDC1 tagged with an OLLAS epitope tag (ARRDC1-OLLAS), which lacks the Tat peptide, produced ARMMS containing ARRDC1-Tat or ARRDC1-OLLAS, respectively. The Western blots (FIG. 5) show that plasmid DNA encoding GFP alone or TAR fused to GFP (TAR-GFP) were both capable of expressing GFP protein in cells transfected with the plasmid DNA.

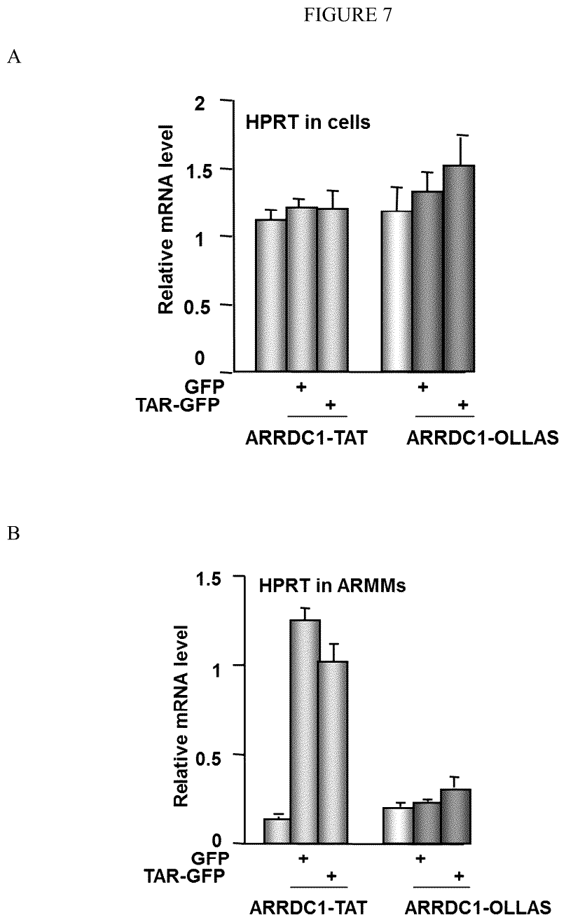

Furthermore, TAR-GFP mRNA was more efficiently packaged into ARMMs using the Tat/TAR system. The relative amount of GFP mRNA detected in ARMMs as compared to their respective ARMM producing cells was significantly increased when ARRDC1-Tat and TAR-GFP were co-expressed in cells as compared to cells that co-expressed ARRDC1-OLLAS and GFP; ARRDC1-OLLAS and TAR-GFP; or ARRDC1-Tat and GFP ARRDC1-OLLAS. See FIG. 6. The relative levels of control, hypoxanthine-guanine phosphoribosyltransferase (HPRT), mRNA in ARMM producing cells that express combinations of GFP and ARRDC1-Tat; GFP and ARRDC1-OLLAS; TAR-GFP and ARRDC1-Tat; TAR-GFP and ARRDC1-OLLAS or a control that does not express any of the constructs, are shown in FIG. 7A. The relative levels of control, (HPRT), mRNA in ARMMs from ARMM producing cells that express combinations of GFP and ARRDC1-Tat; GFP and ARRDC1-OLLAS; TAR-GFP and ARRDC1-Tat; TAR-GFP and ARRDC1-OLLAS or a control that does not express any of the constructs, are shown in FIG. 7B.

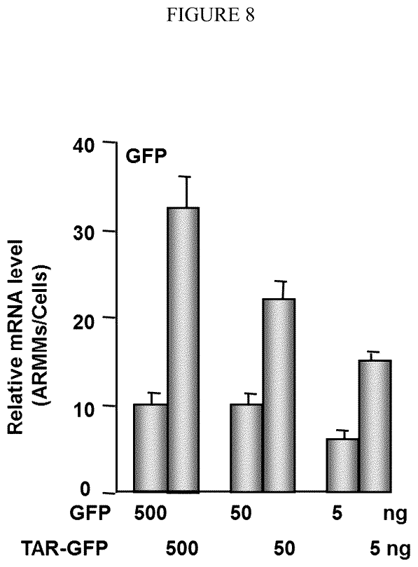

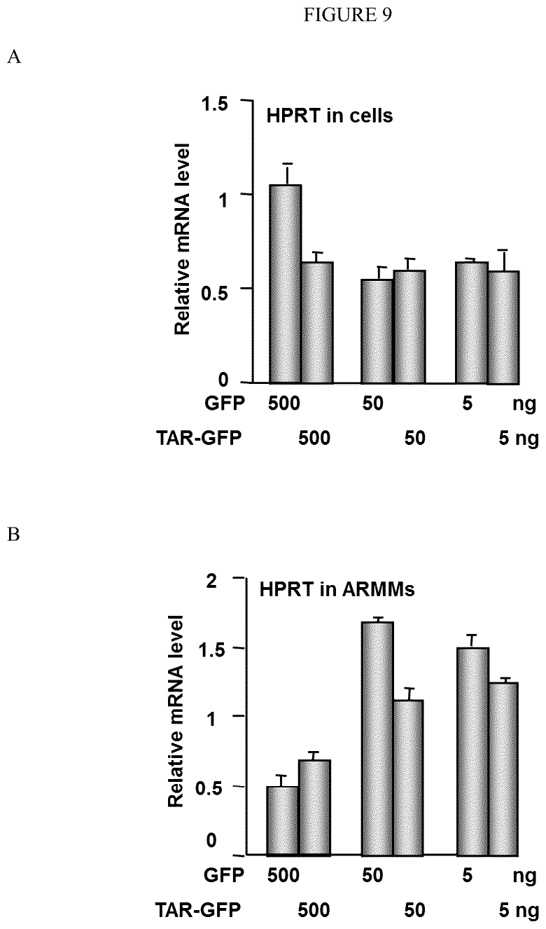

TAR-GFP mRNA was efficiently packaged into ARMMs in a dose-dependent manner. The relative amount of GFP mRNA detected in ARMMs as compared to their respective ARMM producing cells increased in a dose dependent manner for cells co-expressing TAR-GFP and ARRDC1-Tat, but not in cells co-expressing GFP and ARRDC1-Tat (FIG. 8). The amounts of GFP or TAR-GFP transfected into cells was 500 ng, 50 ng and 5 ng, respectively. The relative levels of HPRT control mRNA in ARMM producing cells that were transfected with 500 ng, 50 ng or 5 ng of either GFP or TAR-GFP, respectively, are shown in FIG. 9A. The relative levels of HPRT control mRNA in ARMMs from ARMM producing cells that were transfected with 500 ng, 50 ng or 5 ng of either GFP or TAR-GFP, respectively, are shown in FIG. 9B.

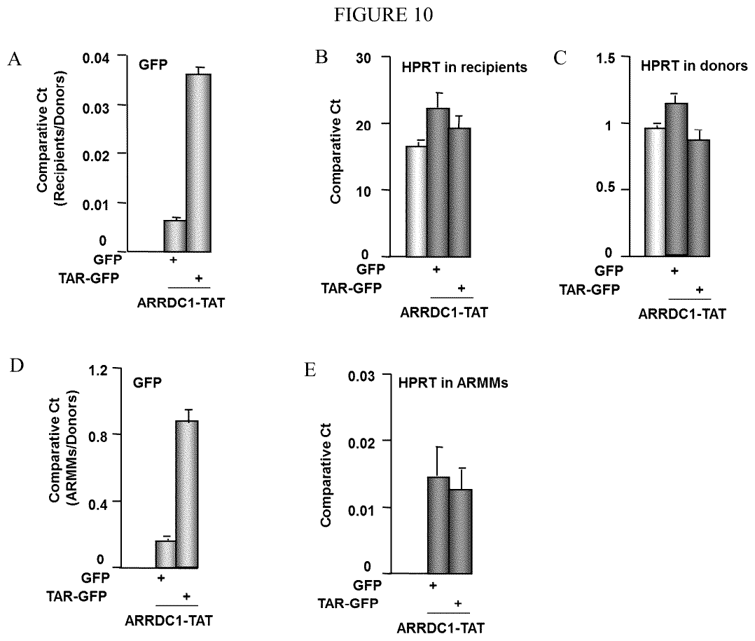

ARMMs containing TAR-GFP mRNA were capable of delivering the TAR-GFP mRNA to a target cells in vitro. The relative amount of GFP mRNA delivered to recipient cells was greater when using ARMMs containing ARRDC1-Tat and TAR-GFP as compared to ARMMs containing ARRDC1-Tat and GFP alone (FIG. 10A). The relative levels of HPRT control mRNA are shown for recipient cells (FIG. 10B) and for donor ARMM producing cells in (1° C.). The relative amount of GFP mRNA in ARMMs was greater in ARMMs produced from donor cells expressing ARRDC1-Tat and TAR-GFP as compared to ARMMs produced from donor cells expressing ARRDC1-Tat and GFP alone (FIG. 10D). The relative levels of HPRT control mRNA in ARMMs produced from donor cells expressing ARRDC1-Tat and TAR-GFP, or ARRDC1-Tat and GFP are shown in FIG. 10E.

### Example 2: Packaging and Delivery of RNAs (e.g., mRNA Encoding p53) Via ARMMs

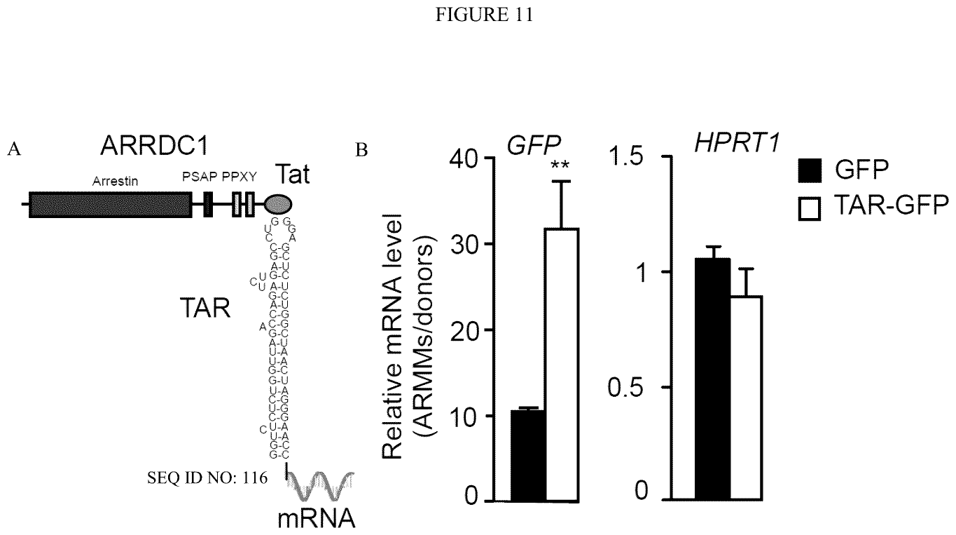

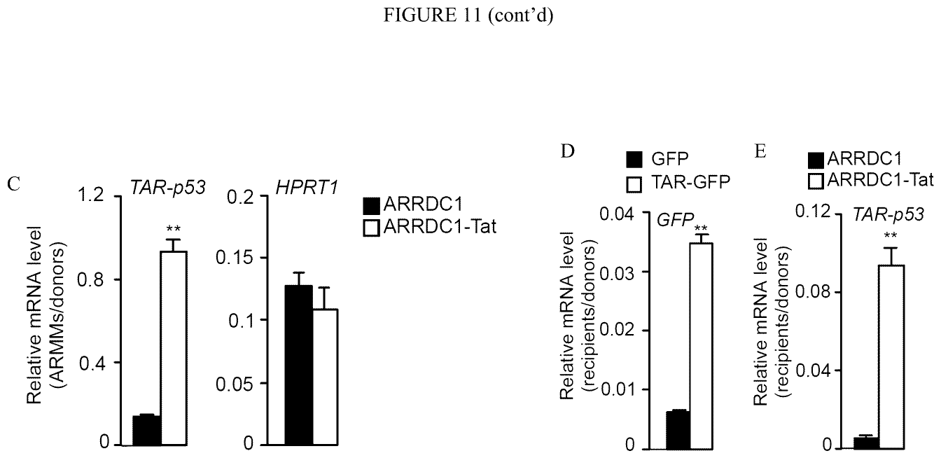

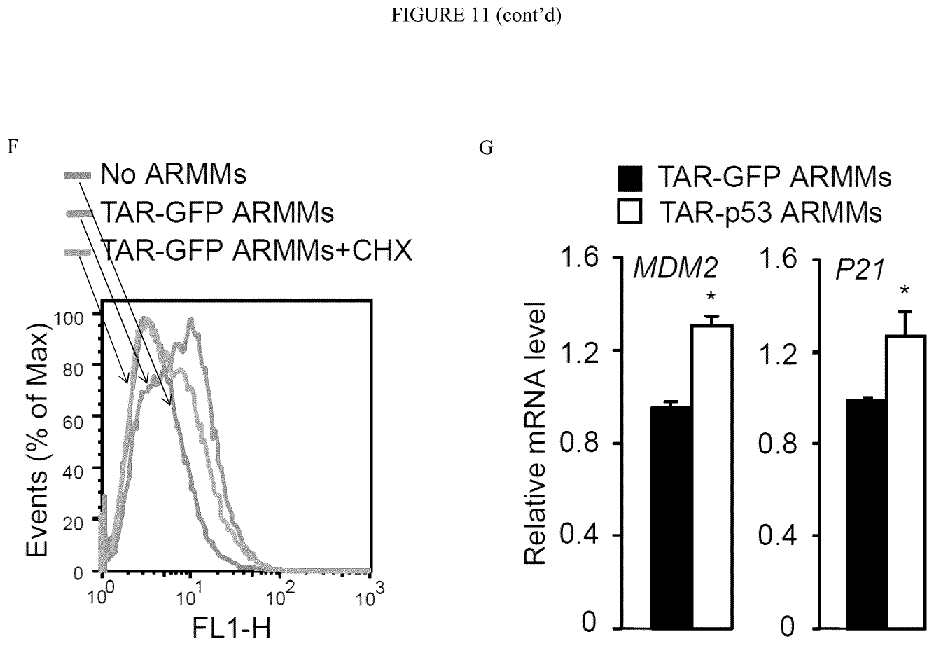

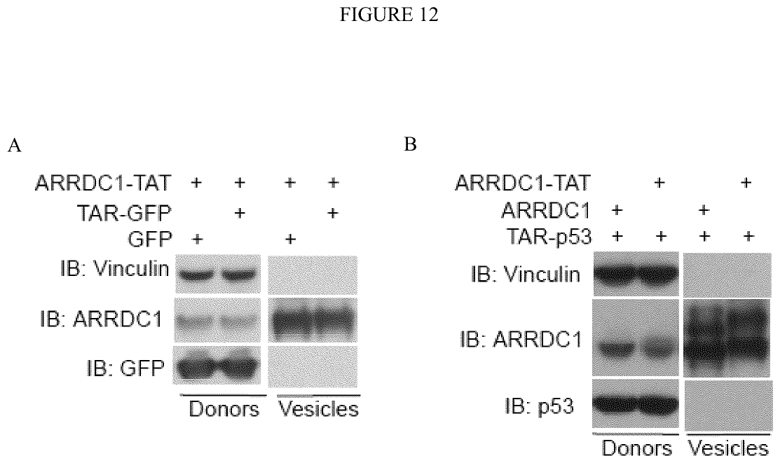

RNA molecules may be broadly used as therapeutic agents (Kole, R., et al., “RNA therapeutics: beyond RNA interference and antisense oligonucleotides.” Nature reviews. Drug discovery 11, 125-140, doi:10.1038/nrd3625 (2012); the contents of which are hereby incorporated by reference in their entirety), but often have to overcome cellular barriers (Dowdy, S. F. “Overcoming cellular barriers for RNA therapeutics.” Nature biotechnology 35, 222-229, doi:10.1038/nbt.3802 (2017); the contents of which are hereby incorporated by reference in their entirety). Accordingly, the ability of ARMMs to package and deliver RNAs to recipient cells was tested. To package RNAs into ARMMs, advantage was taken of the Tat (transactivator of transcription) protein, which binds specifically to the stem-loop-containing TAR (Trans-activating Response element) RNA (Roy, S., et al., “A bulge structure in HIV-1 TAR RNA is required for Tat binding and Tat-mediated trans-activation.” Genes & development 4, 1365-1373 (1990); and Weeks, K. M. et al., “RNA binding assays for Tat-derived peptides: implications for specificity.” Biochemistry 31, 10281-10287 (1992); the contents of each of which are hereby incorporated by reference in their entirety). An expression construct was made with a short Tat peptide fused directly to the C-terminus of ARRDC1 and another construct with TAR fused directly to the 5′ end of a cargo mRNA (FIG. 11A). It was reasoned that the high binding affinity between the Tat peptide and TAR will allow the recruitment of the TAR-fused mRNA into ARMMs. The packaging efficiency of both GFP and p53 mRNAs into ARMMs was tested. Either pcDNA3 backbone construct, ARRDC1-Tat with control GFP, or ARRDC1-Tat with TAR-GFP was transfected into production cells, and harvested ARMMs for mRNA and protein analysis. GFP mRNAs were significantly more enriched in ARMMs of ARRDC1-Tat and TAR-GFP co-transfection (FIG. 11B). Similarly p53 mRNA fused to TAR was significantly enriched in ARMMs when co-expressed with ARRDC1-Tat (FIG. 11C). No GFP or p53 proteins were detected by Western blot in either GFP or TAR-GFP-mRNA-containing ARMMs (FIG. 12), indicating that the Tat-TAR system selectively packaged TAR-labeled mRNAs into ARMMs. It was next determined whether the TAR-GFP (or TAR-p53) mRNA in ARMMs can be delivered into and expressed in recipient cells. Incubation of ARMMs containing TAR-fused mRNAs with recipient A549 cells led to detection of GFP or p53 mRNAs in the recipient cells (FIGS. 11D and 11E). Importantly, flow cytometry analysis confirmed that GFP mRNAs in the recipient cells were translated into GFP proteins and this translation was nearly abolished in the presence of translation inhibitor cycloheximide (CHX) (FIG. 11F). Incubation of ARMMs containing TAR-p53 increased transcription of Mdm2 and p21 in the recipient cells (FIG. 11G), indicating that TAR-p53 mRNAs delivered via ARMMs were translated into functional p53 proteins.

### Plasmids:

To generate ARRDC1-Tat construct, The DNA sequence of ARRDC1 was PCR amplified followed by insertion into pcDNA3 vector to obtain pcDNA3 ARRDC1 construct. The DNA sequence of Tat (48-65 aa) was synthesized, annealed and inserted at the C-terminus of ARRDC1. The DNA sequence of TAR (1-63 base pairs) was synthesized, annealed, and inserted at the 5′ end of EGFP in the pEGFP-N1 vector (Addgene) to obtain the TAR-EGFP construct. Alternatively, the TAR region was inserted at the 5′ end of p53 in the pcDNA3 p53 construct to obtain the TAR-p53 construct.

### REFERENCES

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### Equivalents and Scope

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. (canceled)

2. An arrestin domain-containing protein 1 (ARRDC1)-mediated microvesicle (ARMM) comprising:
a lipid bilayer;
an ARRDC1 protein or variant thereof, wherein the ARRDC1 protein variant comprises a PSAP motif, a PPXY motif, or both, and wherein the ARRDC1 protein variant comprises an amino acid sequence that is at least about 80% identical to the amino acid sequence of any of any of SEQ ID NOs: 15-17;
an RNA binding protein fused to at least one WW domain or variant thereof; and
a binding RNA, wherein the binding RNA is associated with the RNA binding protein.

3. (canceled)

4. The microvesicle of claim 16, wherein at least one WW domain is derived from a WW domain of ubiquitin ligase WWP1, WWP2, Nedd4-1, Nedd4-2, Smurf1, Smurf2, ITCH, NEDL1, or NEDL2.

5. (canceled)

6. The microvesicle of claim 16, further comprising a TSG101 protein or variant thereof.

7.-10. (canceled)

11. The microvesicle of claim 16, wherein the RNA binding protein and the binding RNA are selected from any one of the following pairs:
(i) a trans-activator of transcription (Tat) protein or variant thereof, and a trans-activating response element (TAR) or variant thereof;
(ii) a Rev protein or variant thereof, and a Rev response element (RRE) or variant thereof;
(iii) an MS2 phage coat protein or variant thereof, and an MS2 RNA sequence or variant thereof;
(iv) a P22 N protein or variant thereof, and a P22 boxB RNA sequence or variant thereof;
(v) a λ N protein or variant thereof, and a λ boxB RNA sequence or variant thereof;
(vi) a φ21 protein or variant thereof, and a φ21 boxB RNA sequence or variant thereof; or
(vii) a HIV-1 nucleocapsid protein or variant thereof, and a SL3 ψ RNA sequence or variant thereof.

12.-15. (canceled)

16. The microvesicle of claim 2, wherein the binding RNA is further associated with a cargo RNA.

17. The microvesicle of claim 16, wherein the binding RNA is covalently linked to the cargo RNA.

18. The microvesicle of claim 16, wherein the binding RNA is non-covalently associated with the cargo RNA.

19. The microvesicle of claim 18, wherein the binding RNA is linked to the cargo RNA via complementary base pairing.

20. The microvesicle of claim 16, wherein the binding RNA and the cargo RNA are linked via a linker.

21. The microvesicle of claim 20, wherein the linker is a cleavable linker.

22. The microvesicle of claim 16, wherein the cargo RNA is a messenger RNA (mRNA), a ribosomal RNA (rRNA), a signal recognition particle RNA (SRP RNA), or a transfer RNA (tRNA), a small nuclear RNA (snRNA), a small nucleolar (snoRNA), a SmY RNA (smY), a guide RNA (gRNA), a ribonuclease P (RNase P), a ribonuclease MRP (RNase MRP), a Y RNA, a telomerase RNA component (TERC), a spliced leader RNA (SL RNA), an antisense RNA (asRNA), a cis-natural antisense sequence (cis-NAT), a CRISPR RNA (crRNA), a long noncoding RNA (lncRNA), a microRNA (miRNA), a piwi-interacting RNA (piRNA), a small interfering RNA (siRNA), or a trans-acting siRNA (tasiRNA).

23.-24. (canceled)

25. An ARRDC1 fusion protein comprising:
an ARRDC1 protein or a variant thereof or variant thereof, wherein the ARRDC1 protein variant comprises a PSAP motif, a PPXY motif, or both, and wherein the ARRDC1 protein variant comprises an amino acid sequence that is at least about 80% identical to the amino acid sequence of any of any of SEQ ID NOs: 15-17, and
an RNA binding protein.

26. The ARRDC1 fusion protein of claim 25, wherein the RNA binding protein is associated with a binding RNA.

27. (canceled)

28. The fusion protein of claim 26 further comprising a cargo RNA associated with the binding RNA.

29.-30. (canceled)

31. The fusion protein of claim 26, wherein the cargo RNA comprises a messenger RNA (mRNA), a ribosomal RNA (rRNA), a signal recognition particle RNA (SRP RNA), a transfer RNA (tRNA), a small nuclear RNA (snRNA), a small nucleolar (snoRNA), a SmY RNA (smY), a guide RNA (gRNA), a ribonuclease P (RNase P), a ribonuclease MRP (RNase MRP), a Y RNA, a telomerase RNA component (TERC), a spliced leader RNA (SL RNA), an antisense RNA (asRNA), a cis-natural antisense sequence (cis-NAT), a CRISPR RNA (crRNA), a long noncoding RNA (lncRNA), a microRNA (miRNA), a piwi-interacting RNA (piRNA), a small interfering RNA (siRNA), or a trans-acting siRNA (tasiRNA).

32.-34. (canceled)

35. A nucleic acid construct encoding the ARRDC1 fusion protein of claim 26.

36. A microvesicle-producing cell comprising:
a first recombinant expression construct encoding an ARRDC1 protein or a variant thereof under the control of a heterologous promoter, wherein the ARRDC1 protein variant comprises a PSAP motif, a PPXY motif, or both, and wherein the ARRDC1 protein variant comprises an amino acid sequence that is at least about 80% identical to the amino acid sequence of any of any of SEQ ID NOs: 15-17;
an RNA binding protein or variant thereof, wherein the RNA binding protein is encoded on the first recombinant expression construct and is linked to the ARRDC1 protein or variant thereof, or the RNA binding protein is encoded on the first recombinant expression construct or a second recombinant expression construct and is capable of associating with the ARRDC1 protein or the variant thereof;
a binding RNA, wherein the binding RNA is encoded on the first, the second, or a third recombinant expression construct and is capable of associating with the RNA binding protein; and
a cargo RNA, wherein the cargo RNA is encoded on the first, the second, the third, or a fourth recombinant expression construct and is capable of associating with the RNA binding protein.

37. A method of delivering a cargo RNA to a target cell, the method comprising contacting the target cell with the microvesicle of claim 16.

38. (canceled)

39. A method of altering the expression of at least one gene, the method comprising contacting the target cell with the microvesicle of claim 16.

40.-42. (canceled)

43. A pharmaceutical composition comprising the microvesicle of claim 16 and a pharmaceutically acceptable carrier.

44. (canceled)
